(19) United States
(12) Patent Application Publication
(10) Pub. No.: US 2003/0055037 A1
(43) Pub. Date: Mar. 20, 2003

(54) Benzimidazole and indole derivatives as CRF receptor modulators

(76) Inventors: Stephane DeLombaert, Madison, CT (US); Ping Ge, Durham, CT (US); Raymond F. Horvath, Guilford, CT (US); Taeyoung Yoon, Guilford, CT (US)

Correspondence Address: Dike, Bronstein, Roberts & Cushman, Intellectual Property Practice Group, EDWARDS & ANGELL, LLP, P.O. Box 9169, Boston, MA, 02209, US

(21) Appl. No.: 09/972,786

(22) Filed: Oct. 5, 2001

Related U.S. Application Data
(60) Provisional application No. 60/238,713, filed on Oct. 6, 2000.

(51) Int. Cl. A61K 31/553; A61K 31/554; A61K 31/55; A61K 31/541; A61K 31/5377; A61K 31/496; A61K 31/454; A61K 31/404

(52) U.S. Cl. 514/211.15; 514/212.08; 514/217.09; 514/218; 514/227.8; 514/233.5; 514/254.06; 514/254.09; 514/322; 514/323; 514/394; 514/419

(57) ABSTRACT

Benzimidazole and indole derivatives that act as selective modulators of CRF 1 receptors are provided. These compounds are useful in the treatment of a number of CNS and periphereal disorders, particularly stress, anxiety, depression, cardiovascular disorders, and eating disorders. Methods of treatment of such disorders and well as packaged pharmaceutical compositions are also provided.

Compounds of the invention are also useful as probes for the localization of CRF receptors and as standards in assays for CRF receptor binding. Methods of using the compounds in receptor localization studies are given.

This application claims the benefit of U.S. Provisional Application Ser. No. 60/238,713 filed Oct. 6, 2000, the teachings of which are incorporated herein by reference.

### FIELD OF THE INVENTION

The present invention relates to novel benzimidazole and indole compounds that bind with high selectivity and/ or high affinity to CRF receptors (Corticotropin Releasing Factor Receptors). This invention also relates to pharmaceutical compositions comprising such compounds and to the use of such compounds in treatment of psychiatric disorders and neurological diseases, including major depression, anxiety-related disorders, post-traumatic stress disorder, supranuclear palsy and feeding disorders, as well as treatment of immunological, cardiovascular or heart-related diseases and colonic hypersensitivity associated with psychopathological disturbance and stress. Additionally this invention relates to the use such compounds as probes for the localization of CRF receptors in cells and tissues. Preferred CRF receptors are CRF1 receptors.

### BACKGROUND OF THE INVENTION

Corticotropin releasing factor (CRF), a 41 amino acid peptide, is the primary physiological regulator of proopiomelanocortin (POMC) derived peptide secretion from the anterior pituitary gland. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in brain. There is also evidence that CRF plays a significant role in integrating the response of the immune system to physiological, psychological, and immunological stressors.

Clinical data provide evidence that CRF has a role in psychiatric disorders and neurological diseases including depression, anxiety-related disorders and feeding disorders. A role for CRF has also been postulated in the etiology and pathophysiology of Alzheimer's disease, Parkinson's disease, Huntington's disease, progressive supranuclear palsy and amyotrophic lateral sclerosis as they relate to the dysfunction of CRF neurons in the central nervous system.

In affective disorder, or major depression, the concentration of CRF is significantly increased in the cerebral spinal fluid (CSF) of drug-free individuals. Furthermore, the density of CRF receptors is significantly decreased in the frontal cortex of suicide victims, consistent with a hypersecretion of CRF. In addition, there is a blunted adrenocorticotropin (ACTH) response to CRF (i.v. administered) observed in depressed patients. Preclinical studies in rats and non- human primates provide additional support for the hypothesis that hypersecretion of CRF may be involved in the symptoms seen in human depression. There is also preliminary evidence that tricyclic antidepressants can alter CRF levels and thus modulate the numbers of CRF receptors in brain.

CRF has also been implicated in the etiology of anxiety-related disorders. CRF produces anxiogenic effects in animals and interactions between benzodiazepine / non-benzodiazepine anxiolytics and CRF have been demonstrated in a variety of behavioral anxiety models. Preliminary studies using the putative CRF receptor antagonist alpha-helical ovine CRF (9-41) in a variety of behavioral paradigms demonstrate that the antagonist produces “anxiolytic-like” effects that are qualitatively similar to the benzodiazepines. Neurochemical, endocrine and receptor binding studies have all demonstrated interactions between CRF and benzodiazepine anxiolytics providing further evidence for the involvement of CRF in these disorders. Chlordiazepoxide attenuates the “anxiogenic” effects of CRF in both the conflict test and in the acoustic startle test in rats. The benzodiazepine receptor antagonist Ro 15-1788, which was without behavioral activity alone in the operant conflict test, reversed the effects of CRF in a dose-dependent manner, while the benzodiazepine inverse agonist FG 7142 enhanced the actions of CRF.

CRF has also been implicated in the pathogeneisis of certain immunological, cardiovascular or heart-related diseases such as hypertension, tachycardia and congestive heart failure, stroke and osteoporosis, as well as in premature birth, psychosocial dwarfism, stress-induced fever, ulcer, diarrhea, post-operative ileus and colonic hypersensitivity associated with psychopathological disturbance and stress.

The mechanisms and sites of action through which conventional anxiolytics and antidepressants produce their therapeutic effects remain to be fully elucidated. It has been hypothesized however, that they are involved in the suppression of CRF hypersecretion that is observed in these disorders. Of particular interest are that preliminary studies examining the effects of a CRF receptor antagonist peptide (alpha-helical CRF₉₋₄₁) in a variety of behavioral paradigms have demonstrated that the CRF antagonist produces “anxiolytic-like” effects qualitatively similar to the benzodiazepines.

### SUMMARY OF THE INVENTION

The invention provides novel compounds of Formula I (shown below), and pharmaceutical compositions comprising compounds of Formula I and at least one pharmaceutically acceptable carrier or excipient. Such compounds bind to cell surface receptors, preferably G-coupled protein receptors, especially CRF receptors (including CRF1 and CRF2 receptors) and most preferably CRF 1 receptors. Preferred compounds of the invention exhibit high affinity for CRF receptors, preferably CRF 1 receptors. Additionally, preferred compounds of the invention also exhibit high specificity for CRF receptors (i.e., they exhibit high selectivity compared to their binding to non-CRF receptors). Preferably they exhibit high specificity for CRF 1 receptors.

The present invention provides compounds according to Formula I:

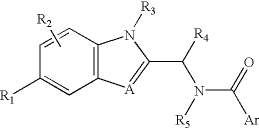

Formula I

or a pharmaceutically acceptable salt thereof, wherein

A is nitrogen, optionally substituted CH, or optionally substituted alkyl;

R₁ is hydrogen, optionally substituted alkyl, optionally substituted haloalkoxy, optionally substituted haloalkyl, halogen, hydroxy, cyano, amino, nitro, XR_{A}, C₁-C₂alkyl-XR_{A}, Y or C₁-C₂alkyl-Y;

R₂ represents from 0 to 3 substituents independently selected from halogen, hydroxy, cyano, amino, optionally substituted alkyl, optionally substituted alkoxy, optionally substituted haloalkyl, optionally substituted haloalkoxy, optionally substituted hydroxyalkyl, and optionally substituted mono- or di-alkylamino;

R₃ represents an optionally substituted alkyl group; or

R₃ represents a saturated, partially unsaturated, or aromatic ring having from 5 to 7 ring atoms, 0, 1, or 2 ring atoms chosen from oxygen and nitrogen, with remaining ring atoms being carbon, which saturated, partially unsaturated, or aromatic ring is unsubstituted or substituted;

R₄ represents hydrogen or optionally substituted alkyl;

R₅ represents optionally substituted alkyl; or

R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon;;

Ar represents phenyl or a heteroaryl group of 5 to 7 ring atoms, 0, 1, or 2 ring atoms chosen from oxygen, nitrogen, and sulfur, with remaining ring atoms being carbon, Ar is substituted ortho to the point of attachment of Ar in Formula I by R₆ and is optionally substituted by 1 or more of R₇;

R₆ represents halogen, hydroxy, cyano, amino, XR_{A}, C₁-C₂alkyl-XR_{A}, or Y;

R₇ is independently selected at each occurrence from hydroxy, cyano, amino, XR_{A}, C₁-C₂alkyl-XR_{A}, and Y;

X is independently selected at each occurrence from the group consisting of a bond, —CH₂—, —CHR_{B}—, —O—, —C(═O)—, —C(═O)O—, —OC(═O)—, —S(O)ₙ—, —NH—, —NR_{B}—, —C(═O)NH—, —C(═O)NR_{B}—, —S(O)ₙNH—, —S(O)ₙNR_{B}—, —NHC(═O)—, —NR_{B}C(═O)—, —NHS(O)_{b}—, and —NR_{B}S(O)ₙ—;

R_{A} and R_{B} are independently selected at each occurrence from:

hydrogen, and optionally substituted straight, branched, and cyclic alkyl groups containing zero or one or more double or triple bonds;

n is independently selected at each occurrence from 0, 1, and 2; and

Y and Z are independently selected at each occurrence from: saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di-(C₁-C₆)alkylamino.

The present invention also provides compounds according to Formula II:

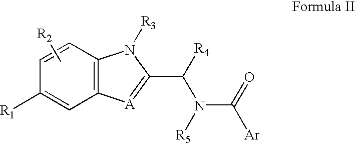

or a pharmaceutically acceptable salt thereof, wherein

A is nitrogen, CH, or C₁-C₆alkyl;

R₁ is hydrogen, optionally substituted alkyl, optionally substituted haloalkoxy, optionally substituted haloalkyl, halogen, hydroxy, cyano, amino, nitro, XR_{A}, C₁-C₂alkyl-XR_{A}, Y or C₁-C₂alkyl-Y;

R₂ represents from 0 to 3 substituents independently selected from halogen, hydroxy, cyano, amino, optionally substituted alkyl, optionally substituted alkoxy, optionally substituted haloalkyl, optionally substituted haloalkoxy, optionally substituted hydroxyalkyl, and optionally substituted mono- or di-alkylamino;

R₃ represents a branched C₃-C₁₀ alkyl group which is optionally substituted by 1 or more substituents independently chosen from halogen, oxo, hydroxy, cyano, amino, C₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; or

R₃ represents a saturated, partially unsaturated, or aromatic ring having from 5 to 7 ring atoms, 0, 1, or 2 ring atoms chosen from oxygen and nitrogen, with remaining ring atoms being carbon, which saturated, partially unsaturated, or aromatic ring is unsubstituted or substituted with one or more substituents independently selected from halogen, oxo, hydroxy, amino, cyano, XR_{A}, C₁-C₆alkyl, C₁-C₆alkyl substituted by XR_{A}, C₁-C₆alkoxy, C₁-C₆alkoxy substituted by XR_{A}, C₁-C₆haloalkyl, C₁-C₆haloalkoxy, mono- or di-(C₁-C₆)alkylamino, and Y;

R₄ represents hydrogen or optionally substituted C₁-C₆ alkyl;

R₅ represents branched C₃-C₁₀ alkyl which is unsubstituted or substituted by 1 to 4 groups independently chosen from hydroxy, cyano, amino, oxo, XR_{A}, and Y; or

R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon; said saturated or partially unsaturated ring is unsubstituted or substituted by 1 to 3 substituents independently chosen from halogen, hydroxy, amino, C₁-C₆alkoxy, C₁-C₆alkyl, C₁-C₆haloalkyl, C₁-C₆haloalkoxy, C₁-C₆hydroxyalkyl, and mono- or di-(C₁-C₆)alkylamino;

Ar represents phenyl or a heteroaryl group of 5 to 7 ring atoms, 0, 1, or 2 ring atoms chosen from oxygen, nitrogen, and sulfur, with remaining ring atoms being carbon, Ar is substituted ortho to the point of attachment of Ar in Formula II by R₆ and is optionally substituted by 1 or more of R₇;

R₆ represents halogen, hydroxy, cyano, amino, XR_{A}, C₁-C₂alkyl-XR_{A}, or Y;

R₇ is independently selected at each occurrence from hydroxy, cyano, amino, XR_{A}, C₁-C₂alkyl-XR_{A}, and Y;

X is independently selected at each occurrence from the group consisting of a bond, —CH₂—, —CHR_{B}—, —O—, —C(═O)—, —C(═O)O—, —OC(═O)—, —S(O)ₙ—, —NH—, —NR_{B}—, —C(═O)NH—, —C(═O)NR_{B}—, —S(O)ₙNH—, —S(O)ₙNR_{B}—, —NHC(═O)—, —NR_{B}C(═O)—, —NHS(O)ₙ—, and —NR_{B}S(O)ₙ—;

R_{A} and R_{B} are independently selected at each occurrence from:

hydrogen, and

straight, branched, and cyclic alkyl groups, and (cycloalkyl)alkyl groups, said straight, branched, and cyclic alkyl groups, and (cycloalkyl)alkyl groups consisting of 1 to 8 carbon atoms, and containing zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;

n is independently selected at each occurrence from 0, 1, and 2; and

Y and Z are independently selected at each occurrence from: saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di-(C₁-C₆)alkylamino.

The present invention also provides compounds according to Formula III:

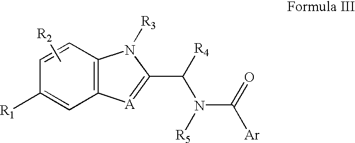

or a pharmaceutically acceptable salt thereof, wherein

A is nitrogen, CH, or C₁-C₆alkyl;

R₁ is hydrogen, halogen, hydroxy, cyano, amino, nitro, XR_{A}, C₁-C₂alkyl-XR_{A}, or Y;

R₂ represents from 0 to 3 substituents independently selected from halogen, hydroxy, cyano, amino, C₁-C₆alkyl, C₁-C₆alkoxy, C₁-C₆haloalkyl, C₁-C₆haloalkoxy, C₁-C₆haloalkoxy, C₁-C₆hydroxyalkyl, and mono- or di-(C₁-C₆)alkylamino;

R₃ represents a branched C₃-C₁₀ alkyl group which is optionally substituted by 1 or more substituents independently chosen from halogen, oxo, hydroxy, cyano, amino, C₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; or

R₃ represents a saturated, partially unsaturated, or aromatic ring having from 5 to 7 ring atoms, 0, 1, or 2 ring atoms chosen from oxygen and nitrogen, with remaining ring atoms being carbon, which saturated, partially unsaturated, or aromatic ring is unsubstituted or substituted with one or more substituents independently selected from halogen, oxo, hydroxy, amino, cyano, XR_{A}, C₁-C₆alkyl, C₁-C₆alkyl substituted by XR_{A}, C₁-C₆alkoxy, C₁-C₆alkoxy substituted by XR_{A}, C₁-C₆haloalkyl, C₁-C₆haloalkoxy, mono- or di-(C₁-C₆)alkylamino, and Y;

R₄ represents hydrogen or optionally substituted C₁-C₆ alkyl;

R₅ represents branched C₃-C₁₀ alkyl which is unsubstituted or substituted by 1 to 4 groups independently chosen from hydroxy, cyano, amino, oxo, XR_{A}, and Y; or

R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon; said saturated or partially unsaturated ring is unsubstituted or substituted by 1 to 3 substituents independently chosen from halogen, hydroxy, amino, C₁-C₆alkoxy, C₁-C₆alkyl, C₁-C₆haloalkyl, C₁-C₆haloalkoxy, C₁-C₆hydroxyalkyl, and mono- or di-(C₁-C₆)alkylamino;

Ar represents phenyl or a heteroaryl group of 5 to 7 ring atoms, 0, 1, or 2 ring atoms chosen from oxygen, nitrogen, and sulfur, with remaining ring atoms being carbon, Ar is substituted ortho to the point of attachment of Ar in Formula III by R₆ and is optionally substituted by 1 or more of R₇;

R₆ represents halogen, hydroxy, cyano, amino, XR_{A}, C₁-C₂alkyl-XR_{A}, or Y;

R₇ is independently selected at each occurrence from hydroxy, cyano, amino, XR_{A}, C₁-C₂alkyl-XR_{A}, and Y;

X is independently selected at each occurrence from the group consisting of a bond, —CH₂—, —CHR_{B}—, —O—, —C(═O)—, —C(═O)O—, —OC(═O)—, —S(O)ₙ—, —NH—, —NR_{B}—, —C(═O)NH—, —C(═O)NR_{B}—, —S(O)ₙNH—, —S(O)ₙNR_{B}—, —NHC(═O)—, —NR_{B}C(═O)—, —NHS(O)ₙ—, and —NR_{B}S(O)ₙ—;

R_{A} and R_{B} are independently selected at each occurrence from:

hydrogen, and

straight, branched, and cyclic alkyl groups, and (cycloalkyl)alkyl groups, said straight, branched, and cyclic alkyl groups, and (cycloalkyl)alkyl groups consisting of 1 to 8 carbon atoms, and containing zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;

n is independently selected at each occurrence from 0, 1, and 2; and

Y and Z are independently selected at each occurrence from: saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di-(C₁-C₆)alkylamino.

Preferred compounds or salts according to Formulae I, II and III include those wherein A is nitrogen.

The invention further comprises methods of treating patients suffering from certain disorders with a therapeutically effective amount of at least one compound of the invention. These disorders include CNS disorders, particularly affective disorders, anxiety disorders, stress-related disorders, eating disorders and substance abuse. The patient suffering from these disorders may be a human or other animal (preferably a mammal), such as a domesticated companion animal (pet) or a livestock animal. Preferred compounds of the invention for such therapeutic purposes are those that antagonize the binding of CRF to CRF receptors (preferably CRF1, or less preferably CRF2 receptors). The ability of compounds to act as antagonists can be measured as an IC₅₀ value as described below.

According to yet another aspect, the present invention provides pharmaceutical compositions comprising compounds of Formulae I, II and/or III or the pharmaceutically acceptable salts (by which term is also encompassed pharmaceutically acceptable solvates) thereof, which compositions are useful for the treatment of the above-recited disorders. The invention further provides methods of treating patients suffering from any of the above-recited disorders with an effective amount of a compound or composition of the invention.

Additionally this invention relates to the use of the compounds of the invention (particularly labeled compounds of this invention) as probes for the localization of receptors in cells and tissues and as standards and reagents for use in determining the receptor-binding characteristics of test compounds.

Preferred benzimidazole and indole compounds of the invention exhibit good activity, i.e., a half-maximal inhibitory concentration (IC₅₀) of less than 1 millimolar, in the standard in vitro CRF receptor binding assay of Example 2, which follows. Particularly preferred benzimidazole and indole compounds of the invention exhibit an IC₅₀ of about 1 micromolar or less, still more preferably an IC₅₀ of about 100 nanomolar or less even more preferably an IC₅₀ of about 10 nanomolar or less. Certain particularly preferred compounds of the invention will exhibit an IC₅₀ of 1 nanomolar or less in such a defined standard in vitro CRF receptor binding assay.

### DETAILED DESCRIPTION OF THE INVENTION

In addition to compounds of Formulae I, II and III, described above, the invention is further directed to compounds and pharmaceutically acceptable salts of Formulae I, II and/or III wherein

A is nitrogen; and R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl.

Other preferred compounds of Formulae I, II and/or III include those compounds and pharmaceutically acceptable salts wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

Ar represents phenyl, pyridyl, pyrimidinyl, thienyl, pyrrolyl, imidazolyl, pyrazolyl, thiazolyl, oxazolyl, or isoxazolyl, each of which is substituted ortho to the point of attachment in Formulae I, II and/or III by R₆ and is optionally substituted by from 1 to 3 of R₇.

Additionally preferred compounds of Formulae I, II and/or III include those wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino; and

Ar represents phenyl, pyridyl, pyrimidinyl, thienyl, pyrrolyl, imidazolyl, pyrazolyl, thiazolyl, oxazolyl, or isoxazolyl, each of which is substituted ortho to the point of attachment in Formulae I, II and/or III by R₆ and is optionally substituted by from 1 to 3 of R₇.

The present invention is also directed to compounds and pharmaceutically acceptable salts according to Formulae I, II and/or III wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₄ represents hydrogen or methyl;

R₅ represents branched C₃-C₁₀ alkyl; and

Ar represents phenyl or pyridyl, each of which is substituted ortho to the point of attachment in Formulae I, II and/or III by R₆ and is optionally substituted by from 1 to 3 of R₇.

The present invention also provides compounds and pharmaceutically acceptable salts of Formulae I, II and/or III wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₄ represents hydrogen or methyl;

R₅ represents branched C₃-C₁₀ alkyl;

Ar represents phenyl or pyridyl, each of which is substituted ortho to the point of attachment in Formulae I, II and/or III by R₆ and is optionally substituted by from 1 to 3 of R₇; wherein: R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

Other preferred compounds provided by the invention according to Formulae I, II and/or III include those wherein:

A is nitrogen;

R₂ represents hydrogen;

R₃ represents a branched C₃-₁₀ alkyl group which is optionally substituted by 1 or more substituents independently chosen from hydroxy, amino, C₁-C₆alkoxy, and mono- or di- (C₁-C₆)alkylamino;

R₄ represents hydrogen or methyl;

R₅ represents branched C₃-C₁₀ alkyl;

Ar represents phenyl or pyridyl, each of which is substituted ortho to the point of attachment in Formulae I, II and/or III by R₆ and is optionally substituted by from 1 to 3 of R₇; wherein:

R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

Still other preferred compounds and pharmaceutically acceptable salts thereof according to Formulae I, II and/or III include compounds and salts wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents phenyl or pyridyl which is optionally substituted by 1 or 2 substituents independently selected from hydroxy, amino, halogen C₁-C₆alkoxy, C₁-C₆alkyl; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; and

Ar represents phenyl, pyridyl, pyrimidinyl, thienyl, pyrrolyl, imidazolyl, pyrazolyl, thiazolyl, oxazolyl, or isoxazolyl, each of which is substituted ortho to the point of attachment in Formulae I, II and/or III by R₆ and is optionally substituted by from 1 to 3 of R₇.

Additionally preferred compounds and pharmaceutically acceptable salts thereof according to Formulae I, II and/or III include compounds and salts wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents phenyl or pyridyl which is optionally substituted by 1 or 2 substituents independently selected from hydroxy, amino, halogen C₁-C₆alkoxy, C₁-C₆alkyl; and mono- or di-(C₁-C₆)alkylamino;

R₄ represents hydrogen or methyl;

R₅ represents branched C₃-C₁₀ alkyl; and

Ar represents phenyl or pyridyl, each of which is substituted ortho to the point of attachment in Formulae I, II and/or III by R₆ and is optionally substituted by from 1 to 3 of R₇.

The invention also provides compounds or pharmaceutically acceptable salts according to Formulae I, II and/or III, wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents phenyl which is optionally substituted by 1 or 2 substituents independently selected from hydroxy, amino, halogen, C₁-C₄alkoxy, and C₁-C₄alkyl;

R₄ represents hydrogen or methyl;

R₅ represents branched C₃-C₁₀ alkyl;

Ar represents phenyl or pyridyl, each of which is substituted ortho to the point of attachment in Formulae I, II and/or III by R₆ and is optionally substituted by from 1 to 3 of R₇; wherein:

R₆ and R₇ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

Other preferred compounds and pharmaceutically acceptable salts thereof according to Formulae I, II and/or III include compounds and salts, wherein:

A is nitrogen;

R₂ represents hydrogen;

R₃ represents phenyl which is optionally substituted at the position para to the point of attachment of R₃ in Formulae I, II and/or III by C₁-C₂alkoxy or C₁-C₂alkyl;

R₄ represents hydrogen or methyl;

R₅ represents branched C₃-C₁₀ alkyl;

Ar represents phenyl or pyridyl, each of which is substituted ortho to the point of attachment in Formulae I, II and/or III by R₆ and is optionally substituted by from 1 to 3 of R₇; wherein:

R₆ and R₇ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

Additionally preferred compounds and pharmaceutically acceptable salts according to Formulae I, II and/or III include compounds or salts wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon; said saturated or partially unsaturated ring is unsubstituted or substituted by 1 to 3 substituents independently chosen from halogen, hydroxy, amino, C₁-C₆alkoxy, C₁-C₆alkyl; C₁-C₆haloalkyl, C₁-C₆haloalkoxy, C₁-C₆hydroxyalkyl, and mono- or di-(C₁-C₆)alkylamino;

Ar represents phenyl or pyridyl, each of which is substituted ortho to the point of attachment in Formulae I, II and/or III by R₆ and is optionally substituted by from I to 3 of R₇.

Yet other preferred compounds and salts of Formulae I, II and/or III include those compounds and salts wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₄ and R₅ are joined to form a saturated ring of from 5 to 8 ring members, said ring members comprising 0 or 1 additional nitrogen atom, with remaining ring members being carbon; said saturated ring is unsubstituted or substituted by 1 to 3 substituents independently chosen from halogen, hydroxy, amino, C₁-C₂alkoxy, C₁-C₂alkyl; C₁-C₂haloalkyl, C₁-C₂haloalkoxy, and mono- or di-(C₁-C₂)alkylamino;

Ar represents phenyl or pyridyl, each of which is substituted ortho to the point of attachment in Formulae I, II and/or III by R₆ and is optionally substituted by from 1 to 3 of R₇; wherein:

R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

The present invention also provides compounds and pharmaceutically acceptable salts according to Formulae I, II and/or III wherein:

A is nitrogen;

R₂ represents hydrogen;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₄ and R₅ are joined to form a saturated ring of from 5 to 7 ring members, said ring members comprising 1 nitrogen atom, with remaining ring members being carbon; said saturated ring is unsubstituted or substituted by 1 to 3 substituents independently chosen from halogen, methyl, and methoxy;

Ar represents phenyl or pyridyl, each of which is substituted ortho to the point of attachment in Formula I by R₆ and is optionally substituted by from 1 to 3 of R₇; wherein:

R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

Additionally preferred compounds and pharmaceutically acceptable salts of the invention according to Fromula I include those compounds and salts wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents phenyl or pyridyl which is optionally substituted by 1 or 2 substituents independently selected from hydroxy, amino, halogen C₁-C₆alkoxy, C₁-C₆alkyl; and mono- or di-(C₁-C₆)alkylamino;

R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon; said saturated or partially unsaturated ring is unsubstituted or substituted by 1 to 3 substituents independently chosen from halogen, hydroxy, amino, C₁-C₆alkoxy, C₁-C₆alkyl; C₁-C₆haloalkyl, C₁-C₆haloalkoxy, C₁-C₆hydroxyalkyl, and mono- or di-(C₁-C₆)alkylamino; and

Ar represents phenyl or pyridyl, each of which is substituted ortho to the point of attachment in Formula I by R₆ and is optionally substituted by from 1 to 3 of R₇.

Still other preferred compounds and pharmaceutically acceptable salts according to Formula I include compounds and salts wherein:

A is nitrogen;

R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;

R₃ represents phenyl which is optionally substituted by 1 or 2 substituents independently selected from hydroxy, amino, halogen, C₁-C₄alkoxy, and C₁-C₄alkyl;

R₄ and R₅ are joined to form a saturated ring of from 5 to 8 ring members, said ring members comprising 0 or 1 additional nitrogen atom, with remaining ring members being carbon; said saturated ring is unsubstituted or substituted by 1 to 3 substituents independently chosen from halogen, hydroxy, amino, C₁-C₂alkoxy, C₁-C₂alkyl; C₁-C₂haloalkyl, C₁-C₂haloalkoxy, and mono- or di-(C₁-C₂)alkylamino;

Ar represents phenyl or pyridyl, each of which is substituted ortho to the point of attachment in Formula I by R₆ and is optionally substituted by from 1 to 3 of R₇; wherein:

R₆ and R₇ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

The present invention provides yet other preferred compounds and pharmaceutically acceptable salts according to Formulae I, II and/or III, wherein:

A is nitrogen;

R₂ represents hydrogen;

R₃ represents phenyl which is optionally substituted at the position para to the point of attachment of R₃ in Formula I by C₁-C₂alkoxy or C₁-C₂alkyl;

R₄ and R₅ are joined to form a saturated ring of from 5 to 7 ring members, said ring members comprising 1 nitrogen atom, with remaining ring members being carbon; said saturated ring is unsubstituted or substituted by 1 to 3 substituents independently chosen from halogen, methyl, and methoxy;

Ar represents phenyl or pyridyl, each of which is substituted ortho to the point of attachment in Formulae I, II and/or III by R₆ and is optionally substituted by from 1 to 3 of R₇; wherein:

R₆ and R₇ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

The present invention provides preferred compounds of Formulae I, II and III wherein the R₁ substituent is selected from:

R₁ represents phenyl or pyridyl each of which is optionally substituted with 1 to 5 R₇; or

R₁ represents C₁-C₂alkyl-Y, amino, mono or di(C₁-C₆alkyl)amino, mono or di(C₁-C₆alkyl)amino-C₁-C₆alkyl each of which may be optionally substituted with one or two C₁-C₆alkyl, C₁-C₆alkoxy, C₁-C₆alkoxyC₁-C₆alkyl, hydroxy or amino; and

Y is selected from saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di- (C₁-C₆)alkylamino.

Other preferred compounds according to Formulae I, II and/or III provided by the present invention include those wherein:

R₁ represents phenyl or pyridyl each of which is optionally substituted with 1 to 3 R₇; or

R₁ represents C₁-C₂alkyl-Y, amino, mono or di(C₁-C₆alkyl)amino, mono or di(C₁-C₆alkyl)amino-C₁-C₄alkyl each of which may be optionally substituted with one or two C₁-C₆alkyl, C₁-C₆alkoxy, C₁-C₆alkoxyC₁-C₆alkyl, hydroxy or amino;

R₃ represents phenyl, which is optionally substituted by 1 or 2 substituents independently, selected from hydroxy, amino, C₁-C₄alkyl, and C₁-C₄alkoxy;

R₅ represents a branched C₃-C₁₀ alkyl group which is optionally substituted by 1 or more substituents independently chosen from hydroxy, amino, C₁-C₆alkoxy, and mono- or di- (C₁-C₆)alkylamino; and

Ar represents phenyl or pyridyl, each of which is substituted ortho to the point of attachment in Formulae I, II and/or III by R₆ and is optionally substituted by from 1 to 3 of R₇; wherein:

R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

Still other preferred compounds according to Formulae I, II and/or III provided by the present invention include those wherein:

R₁ represents phenyl or pyridyl each of which is optionally substituted with 1 to 3 R₇; or

R₁ represents C₁-C₂alkyl-Y, amino, mono or di(C₁-C₆alkyl)amino, mono or di(C₁-C₆alkyl)amino-C₁-C₄alkyl each of which may be optionally substituted with one or two C₁-C₆alkyl, C₁-C₆alkoxy, C₁-C₆alkoxyC₁-C₆alkyl, hydroxy or amino;

R₃ represents phenyl, which is optionally substituted by 1 or 2 substituents independently, selected from hydroxy, amino, C₁-C₄alkyl, and C₁-C₄alkoxy;

R₄ and R₅ are joined to form a saturated ring of from 5 to 7 ring members, said ring members comprising 1 nitrogen atom, with remaining ring members being carbon; said saturated ring is unsubstituted or substituted by 1 to 3 substituents independently chosen from halogen, methyl, and methoxy; and

Ar represents phenyl or pyridyl, each of which is substituted ortho to the point of attachment in Formula I by R₆ and is optionally substituted by from 1 to 3 of R₇; wherein:

R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

Additional preferred compounds according to Formulae I, II and/or III provided by the present invention include those wherein:

R₁ represents phenyl or pyridyl each of which is optionally substituted with 1 to 3 R₇; or

R₁ represents C₁-C₂alkyl-Y, amino, mono or di(C₁-C₆alkyl)amino, mono or di(C₁-C₆alkyl)amino-C₁-C₄alkyl each of which may be optionally substituted with one or two C₁-C₆alkyl, C₁-C₆alkoxy, C₁-C₆alkoxyC₁-C₆alkyl, hydroxy or amino;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₅ represents a branched C₃-C₁₀ alkyl group which is optionally substituted by 1 or more substituents independently chosen from hydroxy, amino, C₁-C₆alkoxy, and mono- or di- (C₁-C₆)alkylamino; and

Ar represents phenyl or pyridyl, each of which is substituted ortho to the point of attachment in Formulae I, II and/or III by R₆ and is optionally substituted by from 1 to 3 of R₇; wherein:

R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

More preferred compounds according to Formulae I, II and/or III provided by the present invention include those wherein:

R₁ represents phenyl or pyridyl each of which is optionally substituted with 1 to 3 R₇; or

R₁ represents C₁-C₂alkyl-Y, amino, mono or di(C₁-C₆alkyl)amino, mono or di(C₁-C₆alkyl)amino-C₁-C₄alkyl each of which may be optionally substituted with one or two C₁-C₆alkyl, C₁-C₆alkoxy, C₁-C₆alkoxyC₁-C₆alkyl, hydroxy or amino;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₄ and R₅ are joined to form a saturated ring of from 5 to 7 ring members, said ring members comprising 1 nitrogen atom, with remaining ring members being carbon; said saturated ring is unsubstituted or substituted by 1 to 3 substituents independently chosen from halogen, methyl, and methoxy; and

Ar represents phenyl or pyridyl, each of which is substituted ortho to the point of attachment in Formulae I, II and/or III by R₆ and is optionally substituted by from 1 to 3 of R₇; wherein:

R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

In another aspect of the invention, the present invention provides compounds according to Formula IV:

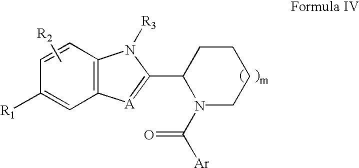

or a pharmaceutically acceptable salt thereof, wherein

A is nitrogen, CH, or C-C₁-C₆alkyl;

R₁ is hydrogen, C₁-C₆alkyl, C₁-C₆haloalkoxy, halogen, hydroxy, cyano, amino, nitro, XR_{A}, C₁-C₂alkyl-XR_{A}, Y or C₁-C₂alkyl-Y;

R₂ represents from 0 to 3 substituents independently selected from halogen, hydroxy, cyano, amino, C₁-C₆alkyl, C₁-C₆alkoxy, C₁-C₆haloalkyl, C₁-C₆haloalkoxy, C₁-C₆hydroxyalkyl, and mono- or di-(C₁-C₆)alkylamino;

R₃ represents a branched C₃-C₁₀ alkyl group which is optionally substituted by 1 or more substituents independently chosen from halogen, oxo, hydroxy, cyano, amino, C₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; or

R₃ represents a saturated, partially unsaturated, or aromatic ring having from 5 to 7 ring atoms, 0, 1, or 2 ring atoms chosen from oxygen and nitrogen, with remaining ring atoms being carbon, which saturated, partially unsaturated, or aromatic ring is unsubstituted or substituted with one or more substituents independently selected from halogen, oxo, hydroxy, amino, cyano, XR_{A}, C₁-C₆alkyl, C₁-C₆alkyl substituted by XR_{A}, C₁-C₆alkoxy, C₁-C₆alkoxy substituted by XR_{A}, C₁-C₆haloalkyl, C₁-C₆haloalkoxy, mono- or di-(C₁-C₆)alkylamino, and Y;

Ar represents phenyl or a heteroaryl group of 5 to 7 ring atoms, 0, 1, or 2 ring atoms chosen from oxygen, nitrogen, and sulfur, with remaining ring atoms being carbon, Ar is substituted ortho to the point of attachment of Ar in Formula IV by R₆ and is optionally substituted by 1 or more of R₇;

R₆ represents halogen, hydroxy, cyano, amino, XR_{A}, C₁-C₂alkyl-XR_{A}, or Y;

R₇ is independently selected at each occurrence from hydroxy, cyano, amino, XR_{A}, C₁-C₂alkyl-XR_{A}, and Y;

X is independently selected at each occurrence from the group consisting of a bond, —CH₂—, —CHR_{B}—, —O—, —C(═O)—, —C(═O)O—, —OC(═O)—, —S(O),—, —NH—, —NR_{B}—, —C(═O)NH—, —C(═O)NR_{B}—, —S(O)ₙNH—, —S(O)ₙNR_{B}—, —NHC(═O)—, —NR_{B}C(═O)—, —NHS(O),—, and —NR_{B}S(O)ₙ—;

R_{A} and R_{B} are independently selected at each occurrence from:

hydrogen, and

straight, branched, and cyclic alkyl groups, and (cycloalkyl)alkyl groups, said straight, branched, and cyclic alkyl groups, and (cycloalkyl)alkyl groups consisting of 1 to 8 carbon atoms, and containing zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;

m is selected from 0, 1, 2, and 3;

n is independently selected at each occurrence from 0, 1, and 2; and

Y and Z are independently selected at each occurrence from: saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di-(C₁-C₆)alkylamino.

Preferred compounds and pharmaceutically acceptable salts according to Formula II include compounds of Formula V:

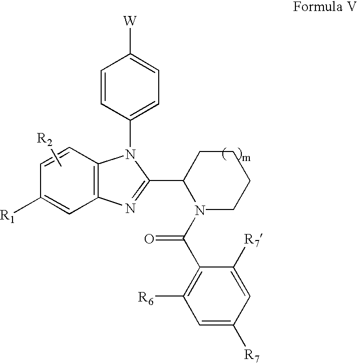

or a pharmaceutically acceptable salt thereof, wherein

R₁ is hydrogen, hydroxy, amino, halogen, C₁-C₆alkoxy, C₁-C₆alkyl, C₁-C₆alkoxyC₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;

R₂ represents from 0 to 3 substituents independently selected from halogen, hydroxy, cyano, amino, C₁-C₂alkyl, C₁-C₂alkoxy, C₁-C₂haloalkyl, C₁-C₂haloalkoxy, and mono- or di-(C₁-C₂)alkylamino;

R₆, R₇, and R₇′ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;

m is O or 1; and

W is methyl, ethyl, methoxy, or ethoxy.

In another aspect of the present invention further provides compounds according to Formula VI:

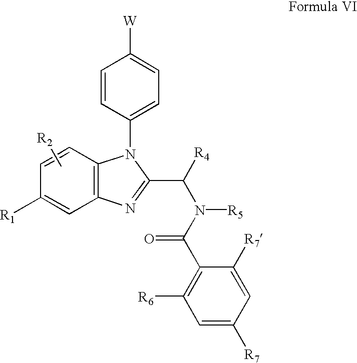

or a pharmaceutically acceptable salt thereof, wherein

R₁ is hydrogen, hydroxy, amino, halogen, C₁-C₆alkoxy, C₁-C₆alkyl, C₁-C₆alkoxyC₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;

R₂ represents from 0 to 3 substituents independently selected from halogen, hydroxy, cyano, amino, C₁-C₂alkyl, C₁-C₂alkoxy, C₁-C₂haloalkyl, C₁-C₂haloalkoxy, and mono- or di-(C₁-C₂)alkylamino;

R₄ is hydrogen or methyl;

R₅ represents branched C₃-C₁₀alkyl; and

R₆, R₇, and R₇′ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

In another aspect of the present invention further provides compounds according to Formula VII:

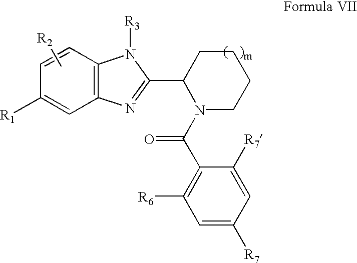

or a pharmaceutically acceptable salt thereof, wherein

R₁ is hydrogen, hydroxy, amino, halogen, C₁-C₆alkoxy, C₁-C₆alkyl, C₁-C₆alkoxyC₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;

R₂ represents from 0 to 3 substituents independently selected from halogen, hydroxy, cyano, amino, C₁-C₂alkyl, C₁-C₂alkoxy, C₁-C₂haloalkyl, C₁-C₂haloalkoxy, and mono- or di-(C₁-C₂)alkylamino;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₆, R₇, and R₇′ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; and

m is O or 1.

In yet another aspect of the present invention further provides compounds according to Formula VIII:

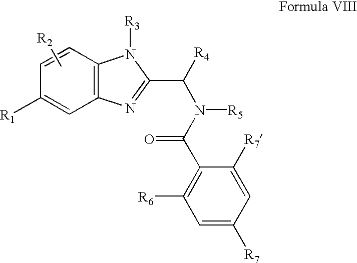

or a pharmaceutically acceptable salt thereof, wherein

R₁ is hydrogen, hydroxy, amino, halogen, C₁-C₆alkoxy, C₁-C₆alkyl, C₁-C₆alkoxyC₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;

R₂ represents from 0 to 3 substituents independently selected from halogen, hydroxy, cyano, amino, C₁-C₂alkyl, C₁-C₂alkoxy, C₁-C₂haloalkyl, C₁-C₂haloalkoxy, and mono- or di-(C₁-C₂)alkylamino;

R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;

R₄ is hydrogen or methyl;

R₅ represents branched C₃-C₁₀alkyl; and

R₆, R₇, and R₇′ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

Compounds of the invention are useful in treating a variety of conditions including affective disorders, anxiety disorders, stress disorders, eating disorders, and drug addiction.

Affective disorders include all types of depression, bipolar disorder, cyclothymia, and dysthymia.

Anxiety disorders include generalized anxiety disorder, panic, phobias and obsessive-compulsive disorder.

Stress-related disorders include post-traumatic stress disorder, hemorrhagic stress, stress-induced psychotic episodes, psychosocial dwarfism, stress headaches, stress-induced immune systems disorders such as stress-induced fever, and stress-related sleep disorders.

Eating disorders include anorexia nervosa, bulimia nervosa, and obesity.

Modulators of the CRF receptors are also useful in the treatment (e.g., symptomatic treatment) of a variety of neurological disorders including supranuclear palsy, AIDS related dementias, multiinfarct dementia, neurodegenerative disorders such as Alzheimer's disease, Parkinson's disease, and Huntington's disease, head trauma, spinal cord trauma, ischemic neuronal damage, amyotrophic lateral sclerosis, disorders of pain perception such as fibromyalgia and epilepsy.

Additionally compounds of Formulae I through VIII are useful as modulators of the CRF receptor in the treatment (e.g., symptomatic treatment) of a number of gastrointestinal, cardiovascular, hormonal, autoimmune and inflammatory conditions. Such conditions include irritable bowel syndrome, ulcers, Crohn's disease, spastic colon, diarrhea, post operative ilius and colonic hypersensitivity associated with psychopathological disturbances or stress, ithypertension, tachycardia, congestive heart failure, infertility, euthyroid sick syndrome, inflammatory conditions effected by rheumatoid arthritis and osteoarthritis, pain, asthma, psoriasis and allergies.

Compounds of Formulae I through VIII are also useful as modulators of the CRF1 receptor in the treatment of animal disorders associated with aberrant CRF levels. These conditions include porcine stress syndrome, bovine shipping fever, equine paroxysmal fibrillation, and dysfunctions induced by confinement in chickens, sheering stress in sheep or human-animal interaction related stress in dogs, psychosocial dwarfism and hypoglycemia.

Typical subjects to which compounds of the invention may be administered will be mammals, particularly primates, especially humans. For veterinary applications, a wide variety of subjects will be suitable, e.g. livestock such as cattle, sheep, goats, cows, swine and the like; poultry such as chickens, ducks, geese, turkeys, and the like; and other domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g., blood, plasma, serum, CSF, lymph, cellular interstitial fluid, aqueous humor, saliva, synovial fluid, feces, or urine) and cell and tissue samples of the above subjects will be suitable for use.

The CRF binding compounds provided by this invention and labeled derivatives thereof are also useful as standards and reagents in determining the ability of test compounds (e.g., a potential pharmaceutical) to bind to a CRF receptor.

Labeled derivatives the CRF antagonist compounds provided by this invention are also useful as radiotracers for positron emission tomography (PET) imaging or for single photon emission computerized tomography (SPECT).

More particularly compounds of the invention may be used for demonstrating the presence of CRF receptors in cell or tissue samples. This may be done by preparing a plurality of matched cell or tissue samples, at least one of which is prepared as an experiment sample and at least one of which is prepared as a control sample. The experimental sample is prepared by contacting (under conditions that permit binding of CRF to CRF receptors within cell and tissue samples) at least one of the matched cell or tissue samples that has not previously been contacted with any compound or salt of the invention with an experimental solution comprising the detectably-labeled preparation of the selected compound or salt at a first measured molar concentration. The control sample is prepared by in the same manner as the experimental sample and is incubated in a solution that contains the same ingredients as the experimental solution but that also contains an unlabelled preparation of the same compound or salt of the invention at a molar concentration that is greater than the first measured molar concentration.

The experimental and control samples are then washed to remove unbound detectably-labeled compound. The amount of detectably-labeled compound remaining bound to each sample is then measured and the amount of detectably-labeled compound in the experimental and control samples is compared. A comparison that indicates the detection of a greater amount of detectable label in the at least one washed experimental sample than is detected in any of the at least one washed control samples demonstrates the presence of CRF receptors in that experimental sample.

The detectably-labeled compound used in this procedure may be labeled with any detectable label, such as a radioactive label, a biological tag such as biotin (which can be detected by binding to detectably-labeled avidin), an enzyme (e.g., alkaline phosphatase, beta galactosidase, or a like enzyme that can be detected its activity in a colorimetric assay) or a directly or indirectly luminescent label. When tissue sections are used in this procedure and the detectably-labeled compound is radiolabeled, the bound, labeled compound may be detected autoradiographically to generate an autoradiogram. When autoradiography is used, the amount of detectable label in an experimental or control sample may be measured by viewing the autoradiograms and comparing the exposure density of the autoradiograms.

The present invention also pertains to methods of inhibiting the binding of CRF to CRF receptors (preferably CFR1 receptors) which methods involve contacting a solution containing a CRF antagonist compound of the invention with cells expressing CRF receptors, wherein the compound is present in the solution at a concentration sufficient to inhibit CRF binding to CRF receptors in vitro. This method includes inhibiting the binding of CRF to CRF receptors in vivo, e.g., in a patient given an amount of a compound of any one of Formulae I through VIII that would be sufficient to inhibit the binding of CRF to CRF receptors in vitro. In one embodiment, such methods are useful in treating physiological disorders associated with excess concentrations of CRF. The amount of a compound that would be sufficient to inhibit the binding of a CRF to the CRF receptor may be readily determined via a CRF receptor binding assay (see, e.g., Example 2), or from the EC₅₀ of a CRF receptor functional assay, such as a standard assay of CRF receptor mediated chemotaxis. The CRF receptors used to determine in vitro binding may be obtained from a variety of sources, for example from cells that naturally express CRF receptors, e.g. IMR32 cells or from cells expressing cloned human CRF receptors.

The present invention also pertains to methods for altering the activity of CRF receptors, said method comprising exposing cells expressing such receptors to an effective amount of a compound of the invention, wherein the compound is present in the solution at a concentration sufficient to specifically alter the signal transduction activity in response to CRF in cells expressing CRF receptors in vitro, preferred cells for this purpose are those that express high levels of CRF receptors (i.e., equal to or greater than the number of CRF1 receptors per cell found in differentiated IMR-32 human neuroblastoma cells), with IMR-32 cells being particularly preferred for testing the concentration of a compound required to alter the activity of CRF1 l receptors. This method includes altering the signal transduction activity of CRF receptors in vivo, e.g., in a patient given an amount of a compound of Formula I that would be sufficient to alter the signal transduction activity in response to CRF in cells expressing CRF receptors in vitro. The amount of a compound that would be sufficient to alter the signal transduction activity in response to CRF of CRF receptors may also be determined via an assay of CRF receptor mediated signal transduction, such as an assay wherein the binding of CRF to a cell surface CRF receptor effects a changes in reporter gene expression.

The present invention also pertains to packaged pharmaceutical compositions for treating disorders responsive to CRF receptor modulation, e.g., eating disorders, depression or stress. The packaged pharmaceutical compositions include a container holding a therapeutically effective amount of at least one CRF1 receptor modulator as described supra and instructions for using the treating disorder responsive to CRF1 receptor modulation in the patient.

### Chemical Description and Terminology

The compounds herein described may have one or more asymmetric centers or planes. Compounds of the present invention containing an asymmetrically substituted atom may be isolated in optically active or racemic forms. It is well known in the art how to prepare optically active forms, such as by resolution of racemic forms (racemates), by asymmetric synthesis, or by synthesis from optically active starting materials. Resolution of the racemates can be accomplished, for example, by conventional methods such as crystallization in the presence of a resolving agent, or chromatography, using, for example a chiral HPLC column. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral (enantiomeric and diastereomeric), and racemic forms, as well as all geometric isomeric forms of a structure are intended, unless the specific stereochemistry or isomeric form is specifically indicated.

When any variable occurs more than one time in any constituent or formula for a compound, its definition at each occurrence is independent of its definition at every other occurrence. Thus, for example, if a group is shown to be substituted with 0-2 R*, then said group may optionally be substituted with up to two R* groups and R* at each occurrence is selected independently from the definition of R*. Also, combinations of substituents and/or variables are permissible only if such combinations result in stable compounds.

Formula I includes, but is not limited to, compounds of Formula I, I, III, IV, V, VI, VII and VIII.

As indicated above, various substituents of the various formulae (compounds of Formula I, II, III, IV, V, VI, VII and VIII) are “optionally substituted”, including Ar, R₁, R₂, R₃, R₄, R₅, R₆, R₇, R₇′, R_{A}, R_{B} and W of Formula I, II, III, IV, V, VI, VII and VII and subformulae thereof, and such substituents as recited in the sub-formulae such as Formula I, II, III, IV, V, VI, VII and VII. The term “substituted,” as used herein, means that any one or more hydrogens on the designated atom or group is replaced with a selection from the indicated group of substituents, provided that the designated atom's normal valence is not exceeded, and that the substitution results in a stable compound. When a substituent is oxo (keto, i.e., ═O), then 2 hydrogens on an atom are replaced. The present invention is intended to include all isotopes (including radioisotopes) of atoms occurring in the present compounds.

When substituents such as Ar, R₁, R₂, R₃, R₄, R₅, R₆, R₇, R₇′, R_{A}, R_{B} and W are further substituted, they may be so substituted at one or more available positions, typically 1 to 3 or 4 positions, by one or more suitable groups such as those disclosed herein. Suitable groups that may be present on a “substituted” Ar, R₁, R₂, R₃, R₄, R₅, R₆, R₇, R₇′, R_{A}, R_{B} and W or other group include e.g., halogen; cyano; hydroxyl; nitro; azido; alkanoyl (such as a C₁-C₆ alkanoyl group such as acyl or the like); carboxamido; alkyl groups (including cycloalkyl groups, having 1 to about 8 carbon atoms, preferably 1, 2, 3, 4, 5, or 6 carbon atoms); alkenyl and alkynyl groups (including groups having one or more unsaturated linkages and from 2 to about 8, preferably 2, 3, 4, 5 or 6, carbon atoms); alkoxy groups having one or more oxygen linkages and from 1 to about 8, preferably 1, 2, 3, 4, 5 or 6 carbon atoms; aryloxy such as phenoxy; alkylthio groups including those having one or more thioether linkages and from 1 to about 8 carbon atoms, preferably 1, 2, 3, 4, 5 or 6 carbon atoms; alkylsulfinyl groups including those having one or more sulfinyl linkages and from 1 to about 8 carbon atoms, preferably 1, 2, 3, 4, 5, or 6 carbon atoms; alkylsulfonyl groups including those having one or more sulfonyl linkages and from 1 to about 8 carbon atoms, preferably 1, 2, 3, 4, 5, or 6 carbon atoms; aminoalkyl groups including groups having one or more N atoms and from 1 to about 8, preferably 1, 2, 3, 4, 5 or 6, carbon atoms; carbocyclic aryl having 6 or more carbons and one or more rings, (e.g., phenyl, biphenyl, naphthyl, or the like, each ring either substituted or unsubstituted aromatic); arylalkyl having 1 to 3 separate or fused rings and from 6 to about 18 ring carbon atoms, with benzyl being a preferred arylalkyl group; arylalkoxy having 1 to 3 separate or fused rings and from 6 to about 18 ring carbon atoms, with O-benzyl being a preferred arylalkoxy group; or a saturated, unsaturated, or aromatic heterocyclic group having 1 to 3 separate or fused rings with 3 to about 8 members per ring and one or more N, O or S atoms, e.g. coumarinyl, quinolinyl, isoquinolinyl, quinazolinyl, pyridyl, pyrazinyl, pyrimidyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such heterocyclic groups may be further substituted, e.g. with hydroxy, alkyl, alkoxy, halogen and amino.

As used herein, “alkyl” is intended to include both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are C₁-C₁₀ alkyl groups. Especially preferred alkyl groups are methyl, ethyl, propyl, butyl, and 3-pentyl. The term C₁₋₄ alkyl as used herein includes alkyl groups consisting of 1 to 4 carbon atoms, which may contain a cyclopropyl moiety. Suitable examples are methyl, ethyl, and cyclopropylmethyl. Especially preferred branched alkyl groups are aliphatic hydrocarbon groups having the specified number of carbon atoms. More preferred are branched alkyl groups where the branching point is at the alpha carbon atom. Particularly preferred branched alkyl groups include 2-propyl, 2-butyl, 2-pentyl, 3-pentyl, 2-hexyl, 3-hexyl, 3-heptyl and 4-heptyl.

“Cycloalkyl” is intended to include saturated ring groups, having the specified number of carbon atoms, such as cyclopropyl, cyclobutyl, cyclopentyl, or cyclohexyl. Cycloalkyl groups typically will have 3 to about 8 ring members.

In the term “(C₃-C₇cycloalkyl)C₁-C₄alkyl”, cycloalkyl, and alkyl are as defined above, and the point of attachment is on the alkyl group. This term encompasses, but is not limited to, cyclopropylmethyl, cyclohexylmethyl, and cyclohexylmethyl.

“Alkenyl” is intended to include hydrocarbon chains of either a straight or branched configuration comprising one or more unsaturated carbon-carbon bonds, which may occur in any stable point along the chain, such as ethenyl and propenyl. Alkenyl groups typically will have 2 to about 8 carbon atoms, more typically 2 to about 6 carbon atoms.

“Alkynyl” is intended to include hydrocarbon chains of either a straight or branched configuration comprising one or more carbon-carbon triple bonds, which may occur in any stable point along the chain, such as ethynyl and propynyl. Alkynyl groups typically will have 2 to about 8 carbon atoms, more typically 2 to about 6 carbon atoms.

“Haloalkyl” is intended to include both branched and straight-chain saturated aliphatic hydrocarbon groups having the specified number of carbon atoms, substituted with 1 or more halogen atoms. Examples of haloalkyl include, but are not limited to, mono-, di-, or tri-fluoromethyl, mono-, di-, or tri-chloromethyl, mono-, di-, tri-, tetra-, or penta-fluoroethyl, and mono-, di-, tri-, tetra-, or penta-chloroethyl. Typical haloalkyl groups will have 1 to about 8 carbon atoms, more typically 1 to about 6 carbon atoms.

“Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Alkoxy groups typically have 1 to about 8 carbon atoms, more typically 1 to about 6 carbon atoms.

“Halolkoxy” represents a haloalkyl group as defined above with the indicated number of carbon atoms attached through an oxygen bridge.

As used herein, the term “alkylthio” includes those groups having one or more thioether linkages and preferably from 1 to about 8 carbon atoms, more typically 1 to about 6 carbon atoms.

As used herein, the term “alkylsulfinyl” includes those groups having one or more sulfoxide (SO) linkage groups and typically from 1 to about 8 carbon atoms, more typically 1 to about 6 carbon atoms.

As used herein, the term “alkylsulfonyl” includes those groups having one or more sulfonyl (SO₂) linkage groups and typically from 1 to about 8 carbon atoms, more typically 1 to about 6 carbon atoms.

As used herein, the term “alkylamino” includes those groups having one or more primary, secondary and/or tertiary amine groups and typically from 1 to about 8 carbon atoms, more typically 1 to about 6 carbon atoms.

“Halo” or “halogen” as used herein refers to fluoro, chloro, bromo, or iodo; and “counter-ion” is used to represent a small, negatively charged species such as chloride, bromide, hydroxide, acetate, sulfate, and the like.

As used herein, “carbocyclic group” is intended to mean any stable 3- to 7-membered monocyclic or bicyclic or 7-to 13-membered bicyclic or tricyclic group, any of which may be saturated, partially unsaturated, or aromatic. In addition to those exemplified elsewhere herein, examples of such carbocycles include, but are not limited to, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, phenyl, naphthyl, indanyl, and tetrahydronaphthyl.

As used herein, the term “heterocyclic group” is intended to include saturated, partially unsaturated, or unsaturated (aromatic) groups having 1 to 3 (preferably fused) rings with 3 to about 8 members per ring at least one ring containing an atom selected from N, O or S. The nitrogen and sulfur heteroatoms may optionally be oxidized. The term or “heterocycloalkyl” is used to refer to saturated heterocyclic groups.

The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. As used herein, the term “aromatic heterocyclic system” is intended to include any stable 5-to 7-membered monocyclic or 10- to 14-membered bicyclic heterocyclic aromatic ring system which comprises carbon atoms and from 1 to 4 heteroatoms independently selected from the group consisting of N, O and S. It is preferred that the total number of S and O atoms in the aromatic heterocycle is not more than 2, more preferably not more than 1.

Examples of heterocycles include, but are not limited to, those exemplified elsewhere herein and further include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl;- 1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

Preferred heterocyclic groups include, but are not limited to, pyridinyl, pyrimidinyl, furanyl, thienyl, pyrrolyl, pyrazolyl, pyrrolidinyl, morpholinyl, piperidinyl, piperazinyl, and imidazolyl. Also included are fused ring and spiro compounds containing, for example, the above heterocycles.

As used herein, the term “carbocyclic aryl” includes groups that contain 1 to 3 separate or fused rings and from 6 to about 18 ring atoms, without hetero atoms as ring members. Specifically preferred carbocyclic aryl groups include phenyl, and naphthyl including 1-napthyl and 2-naphthyl.

As used herein, “pharmaceutically acceptable salts” refer to derivatives of the disclosed compounds wherein the parent compound is modified by making non-toxic acid or base salts thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, malefic, hydroxymaleic, phenylacetic, glutamic, benzoic, salicylic, mesylic, sulfanilic, 2-acetoxybenzoic, fumaric, toluenesulfonic, methanesulfonic, ethane disulfonic, oxalic, isethionic, HOOC—(CH₂)n—COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts may be found, e.g., in Remington's Pharmaceutical Sciences, 17th ed., Mack Publishing Company, Easton, Pa., p. 1418 (1985).

“Prodrugs” are intended to include any compounds that become compounds of Formula I when administered to a mammalian subject, e.g., upon metabolic processing of the prodrug. Examples of prodrugs include, but are not limited to, acetate, formate and benzoate and like derivatives of functional groups (such as alcohol or amine groups) in the compounds of Formula I.

Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. The term “therapeutically effective amount” of a compound of this invention means an amount effective, when administered to a human or non-human patient, to provide a therapeutic benefit such as an amelioration of symptoms, e.g., an amount effective to antagonize the effects of pathogenic levels of CRF or to treat the symptoms of stress disorders, affective disorder, anxiety or depression.

### Pharmaceutical Preparations

The compounds of Formulae I, II, III, IV, V, VI, VII and/or VIII may be administered orally, topically, transdermally, parenterally, by inhalation or spray or rectally or vaginally in dosage unit formulations containing conventional non-toxic pharmaceutically acceptable carriers, adjuvants and vehicles. The term parenteral as used herein includes subcutaneous, intravenous, intramuscular, intrathecal and like types of injection or infusion techniques. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents Iand suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable dilutent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at body temperature and will therefore melt in the body to release the drug. Such materials include cocoa butter and polyethylene glycols.

Compounds of Formulae I, II, III, IV, V, VI, VII and/or VIII may be administered parenterally in a sterile medium. The drug, depending on the vehicle and concentration used, can either be suspended or dissolved in the vehicle. Advantageously, one or more adjuvants such as preservatives, buffering agents, or local anesthetics can also be present in the vehicle.

Dosage levels of the order of from about 0.05 mg to about 100 mg per kilogram of body weight per day are useful in the treatment of the above-indicated conditions, preferred dosages range from about 0.1 to about 30 mg per kg and more preferably from about 0.5 to about 5 mg per kg per subject per day. The amount of active ingredient that may be combined with the carrier materials to produce a single dosage form will vary depending upon the host treated and the particular mode of administration. Dosage unit forms will generally contain between from about 0.1 mg to about 750 mg of an active ingredient.

Frequency of dosage may also vary depending on the compound used and the particular disease treated. However, for treatment of most CNS and gastrointestinal disorders, a dosage regimen of four times daily, preferably three times daily, more preferably two times daily and most preferably once daily is contemplated. For the treatment of stress and depression a dosage regimen of 1 or 2 times daily is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination (i.e. other drugs being used to treat the patient) and the severity of the particular disease undergoing therapy.

Preferred compounds of the invention will have certain pharmacological properties. Such properties include, but are not limited to oral bioavailability, such that the preferred oral dosage forms discussed above can provide therapeutically effective levels of the compound in vivo. Penetration of the blood brain barrier is necessary for most compounds used to treat CNS disorders, while low brain levels of compounds used to treat periphereal disorders are generally preferred.

Assays may be used to predict these desirable pharmacological properties. Assays used to predict bioavailability include transport across human intestinal cell monolayers, including Caco-2 cell monolayers. Toxicity to cultured hepatocyctes may be used to predict compound toxicity, with non-toxic compounds being preferred. Penetration of the blood brain barrier of a i compound in humans may be predicted from the brain levels of the compound in laboratory animals given the compound, e.g., intravenously.

Percentage of serum protein binding may be predicted from albumin binding assays. Examples of such assays are described in a review by Oravcová, et al. (Journal of Chromatography B (1996) volume 677, pages 1-27). Preferred compounds exhibit reversible serum protein binding. Preferably this binding is less than 99%, more preferably less than 95%, even more preferably less than 90%, and most preferably less than 80%.

Frequency of administration is generally inversely proportional to the in vivo half-life of a compound. In vivo half-lives of compounds may be predicted from in vitro assays of microsomal half-life as described by Kuhnz and Gieschen (Drug Metabolism and Disposition, (1998) volume 26, pages 1120-1127). Preferred half lives are those allowing for a preferred frequency of administration.

As discussed above, preferred compounds of the invention exhibit good activity in standard in vitro CRF receptor binding assays, preferably the assay as specified in Example 2, which follows. References herein to “standard in vitro receptor binding assay” are intended to refer to that protocol as defined in Example 2, which follows. Generally preferred compounds of the invention have an IC₅₀ (half-maximal inhibitory concentration) of about 1 micromolar or less, still more preferably and IC₅₀ of about 100 nanomolar or less even more preferably an IC₅₀ of about 10 nanomolar or less or even 1 nanomolar or less in such a defined standard in vitro CRF receptor binding assay as exemplified by Example 2 which follows.

### EXAMPLES

### Preparation of Compounds

The compounds of the present invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include but are not limited to those methods described below. Methods for the preparation of compounds of the present invention include, but are not limited to, those described in the schemes and examples given below. Those who are skilled in the art will recognize that the starting materials may be varied and additional steps employed to produce compounds encompassed by the present invention.

### Abbreviations

The following abbreviations are used in synthetic examples 1-11.

MeOH—methanol LAH—lithium aluminum hydride

HOAc—acetic acid Et₂O—diethyl ether

EtOAc—ethyl acetate PTAB—diethylphosphoryl cyanide

Et₃N—triethylamine THF—tetrahydrofuran

DMF—dimethylformamide DiBAL—diisobutyl aluminum hydride

DEPC—phenyltrimethylammonium SPE—solid phase extraction tribromide

### Example 1

Preparation of N-Isopropyl-2,4,6-trimethyl-N-[1-(1-propylbutyl)-1H-benzoimidazol-2-ylmethyl]benzamide

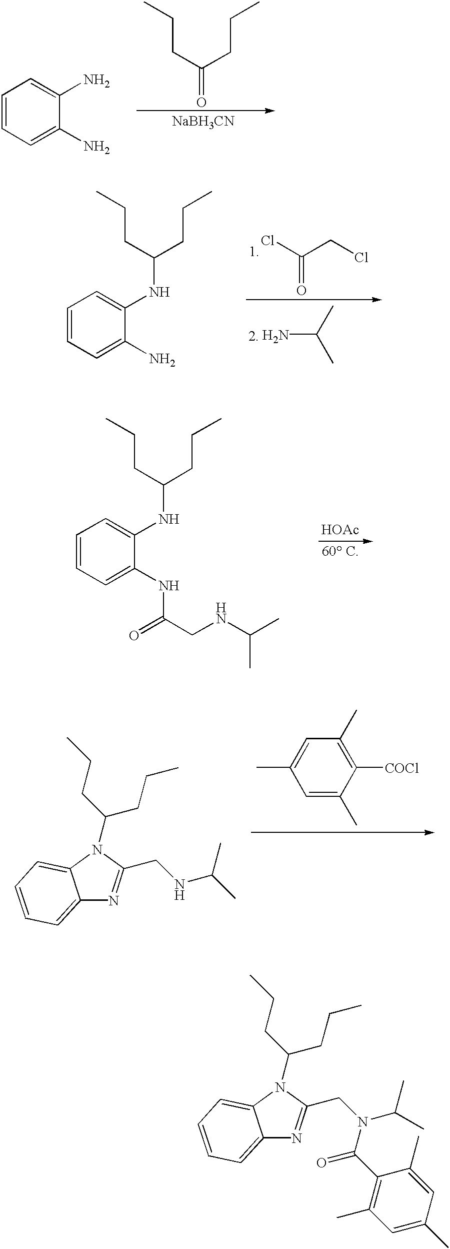

Step 1. 4-heptanone (0.9 ml, 6.4 mmol) is added to a solution of 1,2-phenylenediamine (1.35 g, 12.4 mmol) in MeOH (4 ml), followed by HOAc (acetic acid, 0.2 ml). The resulting mixture is heated to 65° C. and NaBH₃CN (500 mg, 8 mmol) is added. After 30 min, the mixture is cooled to room temperature. and evaporated to dryness. The residue is dissolved in EtOAc, washed with water, brine, dried, filtered and evaporated. The crude product is purified by column chromatograph (eluted with 30% CH₂Cl₂ in hexane) to give the product as a colorless oil. ¹H NMR (CDCl₃): δ0.92 (t, J=6.9 Hz, 6H), 1.30-1.56 (m, 8H), 3.20 (br, 3H), 3.35 (pt, J=5.7 Hz, 1H), 6.60-6.64 (m, 1H), 6.63 (d, J=7.8 Hz, 1H), 6.72 (dd, J=1.8 and 8.1 Hz, 1H), 6.81 (ddd, J=1.5, 7.5 and 9.6 Hz, 1H).

Step 2. Chloroacetyl chloride (0.09 ml, 1.12 mmol) is added to a solution of the above diamine (230 mg, 1.12 mmol) in CH₂Cl₂ (1 ml). at room temperature. The resulting solution is stirred for 30 minutes, and then poured into a solution of isopropylamine (0.95 ml, 11.15 mmol) in CH₂Cl₂ (4 ml) and stirred for 2 hours more. The reaction mixture is evaporated to dryness, dissolved in CH₂Cl₂ and washed with water, brine, dried, filtered and evaporated to give a light yellow oil. ¹H NMR (CDCl₃): δ0.91 (t, J=6.9 Hz, 6H), 1.14 (d, J=6.3 Hz, 6m), 1.30-1.56 (m, 8H), 1.60-1.80 (br, 2H), 2.85 (pt, J=6.3 Hz, 1H), 3.36 (pt, J=5.7Hz, 1H), 3.44 (s, 2H), 6.67-6.72 (m, 2H), 7.05-7.11 (m, 1H), 7.34-7.38(m,1H), 9.18 (s, 1H). LC-MS: 306.2 (M+1).

Step 3. The solution of the above amide in HOAc (2.5 ml) is heated to 60° C. for 10 hours, then evaporated to dryness, dissolved in CH₂Cl₂, washed with saturated NaHCO₃, water, brine, dried, filtered and evaporated to give a light brown oil. ¹H NMR (CDCl₃): δ0.86 (t, J=7.5 Hz, 6H), 1.14 (d, J=6.0 Hz, 6H), 1.20-1.40 (m, 2H), 1.80-1.92 (m, 4H), 2.02-2.18 (m, 211), 2.93 (ht, J=6.3 Hz, 1H), 4.02 (s, 2H), 4.43 (m, 1H), 7.18-7.22 (m, 2H), 7.45-7.49 (m, 1H), 7.71-7.74(m,1H). LC-MS: 288.3 (M+1).

Step 4. Et₃N (0.135 ml, 0.98 mmol) is added to a solution of the above amine (255 mg, 0.887 mmol) in CH₂Cl₂ (1.5 ml) at room temperature, followed by 2,4,6-trimethylbenzoyl chloride (0.147 ml, 0.887 mmol). The resulting mixture is stirred for 2 hours, and then diluted with CH₂Cl₂ (4 ml), washed with water, brine, dried, filtered and evaporated. The crude is purified by column chromatography (eluted with CH₂Cl₂ to 1% MeOH in CH₂Cl₂ ) to give the product as a light yellow oil. ¹H NMR (CDCl₃) showed two conformers in a ratio of about 1 to 3. Peaks derived from the major conformer: δ0.89 (t, J=7.5 Hz, 6H), 1.19 (d, J=6.9 Hz, 6H), 2.25 (s, 3H), 2.33 (s, 6H), 3.90 (m, 1H), 4.72 (m, 1H), 4.98 (s, 211), 6.82 (s, 2H). Peaks derived from the minor conformer: δ0.71 (t, J=7.5 Hz, 6H), 1.38 (d, J=6.6 Hz, 611), 2.14 (s, 3H), 2.28 (s, 6H), 3.80 (m, 1H), 4.45 (s, 2H), 4.95 (m, 1H), 6.65 (s, 2H). Peaks mixed together: 1.58-1.70 (m, 2H), 1.90-2.10 (m, 6H), 7.10-7.25 (m, 2H), 7.50-7.60 (m, 1H), 7.68-7.78 (m, 1H). LC-MS: 434.4 (M+1).

### Example 2

Preparation of N-(2-Hydroxy-1-methylethyl)-N-[1-(4-methoxyphenyl)-5-trifluoromethyl-1H-benzoimidazol-2-yl-methyl]-2,4,6-trimethylbenzamide

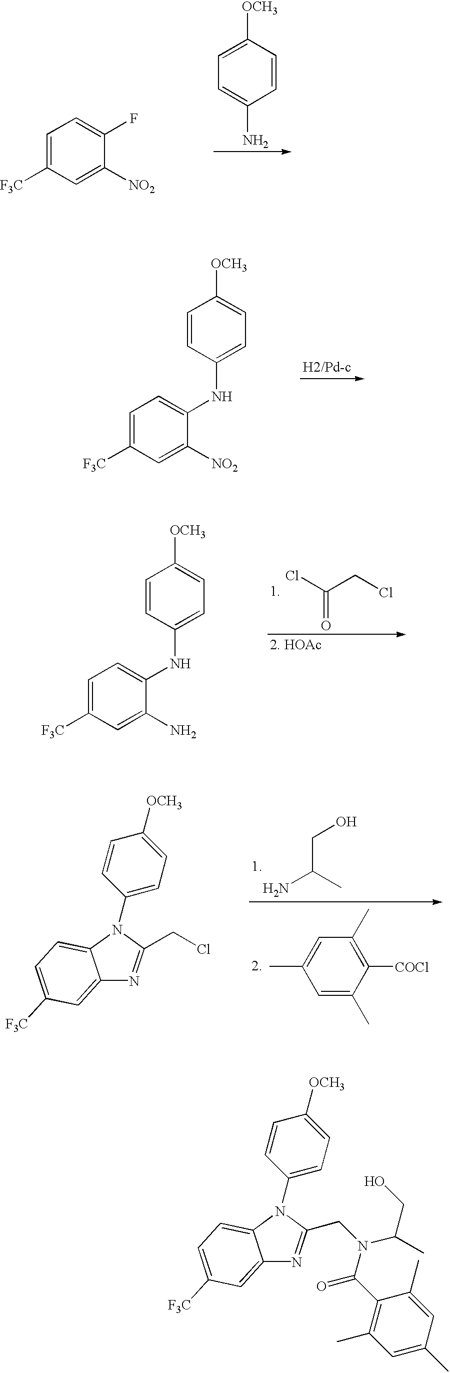

Step 1. 4-fluoro-3-nitrobenzotrifluoride (28 ml, 0.2 mole) is added to a solution of p-anisidine (24.6 g, 0.2 mole) in Et₃N (65 ml, 0.47 mole). The resulting mixture is heated at reflux and kept stirring for 1 hour. After cooling, water (500 ml) is added. The resulting red crystals are collected by filtration, washed thoroughly with water and dried.

Step 2. The above nitro compound (20 g, 64 mmol) is dissolved in EtOH/EtOAc (50 ml/100 ml) and hydrogenated at 30 psi with 10% Pd on carbon (0.7 g) for 2 hours. The catalyst is removed and the light yellow filtrate evaporated to dryness to give the product as a light brown crystalline solid. ¹H NMR (CDCl₃): δ3.65 (br, 2H), 3.80 (s, 3H), 5.24 (br, 1H), 6.86 (d, 2H), 6.91 (d, 2H), 6.98 (m, 3H).

Step 3. Et₃N (0.5 ml, 3.54 mmol) is added to a solution of the above aniline (1.0 g, 3.54 mmol) in 1,2-dichloroethane (10 ml), followed by chloroacetyl chloride (0.28 ml, 3.54 mmol) at 0° C. The resulting mixture is stirred at room temperature overnight, then diluted with CH₂Cl₂ and washed with water, brine, dried, filtered and evaporated to give an oil residue. This residue is dissolved in HOAc (10 ml) and heated to 70° C. for 2 hours then evaporated to dryness. The resulting oil residue is dissolved in 50% EtOAc in hexane and washed with water, brine, dried, filtered and evaporated to give a light yellow oil. ¹H NMR (CDCl₃): δ3.92 (s, 3H), 4.65 (s, 2H), 7.10 (d, 2H), 7.22 (d, 1H), 7.40 (d, 2H), 7.54 (d, 1H), 8.10 (s,1H). LC-MS: 341.1 (M+1).

Step 4. A solution of the above chloride (0.56 g, 1.64 mmol) in CH₃CN (5 ml) is added to a solution of 2-aminopropanol (0.79 ml, 9.86 mmol) in CH₃CN (3 ml). The resulting mixture is stirred overnight, and then evaporated. The residue is dissolved in EtOAc and washed with water, brine, dried, filtered and evaporated. The obtained oil (0.57 g) is dissolved in 1,2-dichloroethane (5 ml). Et₃N (0.26 ml) is added, followed by 2,4,6-trimethylbenzoyl chloride (0.25 ml, 1.5 mmol). The resulting mixture is stirred for 2 hours, then diluted with CH₂Cl₂ (15 ml) and washed with water, brine, dried, filtered and evaporated. The crude is purified by column chromatograph (eluted with 1% MeOH in CH₂Cl₂ ) to give the product as white crystalline solid (460 mg). ¹H NMR (CDCl₃) δ1.06 (d, J=5.1 Hz, 3H), 2.19 (s, 3H), 2.27 (s, 3H), 2.28 (s, 3H), 3.50 (m, 1H), 3.62 (dd, 1H), 3.86 (m, 1H), 3.92 (s, 3H), 4.10 (d, J=12.0 Hz, 1H), 4.92 (d, J=12.0 Hz, 1H), 6.80 (s, 1H), 6.86 (s, 1H), 7.13 (m, 4H), 7.20 (d, J=6.3 Hz, 2H), 7.48 (d, J=6.3 Hz, 1H), 7.55 (br, 1H), 8.01 (s, 1H). LC-MS: 526.5 (M+1).

### Example 3

Preparation of 2-[1-(4-Methoxyphenyl)-1H-benzoimidazol-2-yl]-N-(2,4-dimethoxyphenzoyl)-piperidine

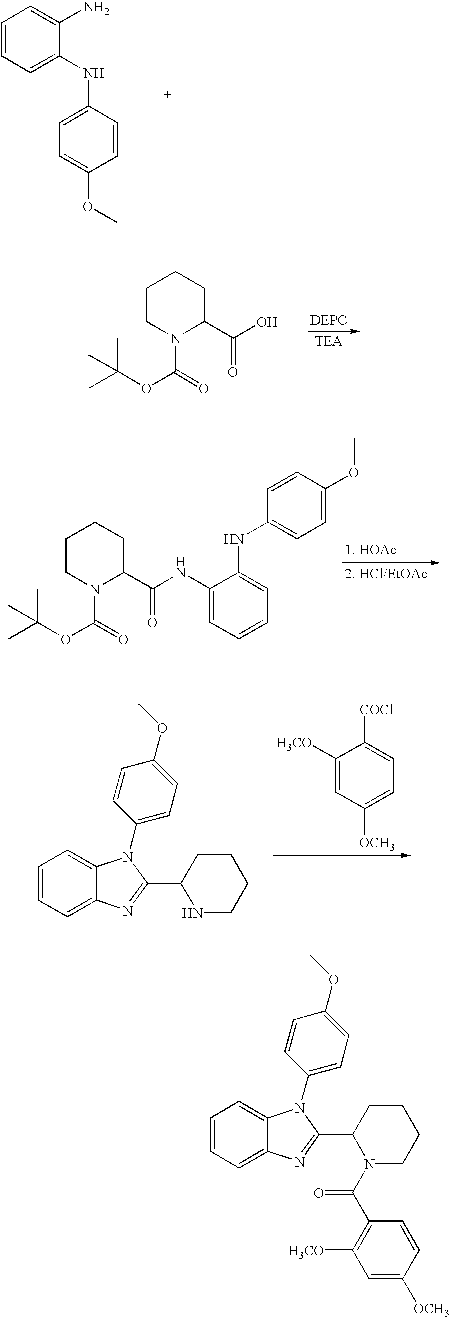

Step 1. Et₃N (1.9 ml, 13.6 mmol) is added to a solution of piperidine-1,2-dicarboxylic acid 1-tert-butyl ester (2.6 g, 11.34 mmol) in DMF (10 ml), followed by DEPC (2.3 ml, 13.6 mmol). The resulting solution is stirred at 0° C. for 20 minutes. N-(4-methoxyphenyl)benzene-1,2-diamine is added and the solution is then warmed to room temperature. After 10 hours, the mixture is diluted with EtOAc, washed with water, then brine, and dried, filtered and evaporated. The crude is purified by column chromatography (eluted with 20% EtOAc in hexane) to give the product as white solid. ¹H NMR (CDCl₃) δ1.40 (s, 9H), 1.50-1.70 (m, 4H), 3.32 (m, 2H), 2.62 (m, 211), 3.78 (s, 3H), 3.95 (m, 1H), 4.85 (br, 1H), 6.80 (s, 4H), 7.04-7.14 (m, 3H), 7.82-7.88 (m, 1H), 8.28 (br, 1H).

Step 2. The above amide (780 mg) is dissolved in HOAc (8 ml), heated to reflux for 2 hours, and then evaporated to dryness. The residue is dissolved in HCl/EtOAc (2N, 10 ml) and stirred for 3 hours. After evaporation, the residue is crystallized from EtOAc/hexane. The crystals are collected, washed with 20% EtOAc in hexane and dried to give the HCl salt as a light yellow crystalline solid. ¹H NMR (CDCl₃) δ1.40 (m, 2H), 1.80-2.02 (m, 4H), 3.05 (m, 1H), 3.45 (s, 2H), 3.85 (s, 3H), 4.40 (m, 1H), 7.08 (m, 2H), 7.20-7.32 (m, 5H), 7.78 (dd, J=1.2 and 6.9 Hz, 1H). LC-MS: 308.2 (M+1).

Step 3. 2,4-dimethoxybenzoyl chloride (60 mg, 0.3 mmol) and Et₃N (0.11 ml, 0.8 mmol) in CH₂Cl₂ (2 ml) is added to a solution of the above hydrochloride (70 mg, 0.2 mmol). The resulting mixture is stirred at room temperature for 3 hours, diluted with CH₂Cl₂, washed with water, saturated NaHCO₃, then brine, and dried, filtered and evaporated. The crude product is purified by preparative TLC (eluted with 2% MeOH in CH₂Cl₂, Rf 1.5) to give the product as white solid. LC-MS: 472.1 (M+1).

### Example 4

Preparation of N-isopropyl-N-[1-(4-methoxyphenyl)-1H-indol-2-yl)methyl]-2,4,6-trimethylbenzamnide

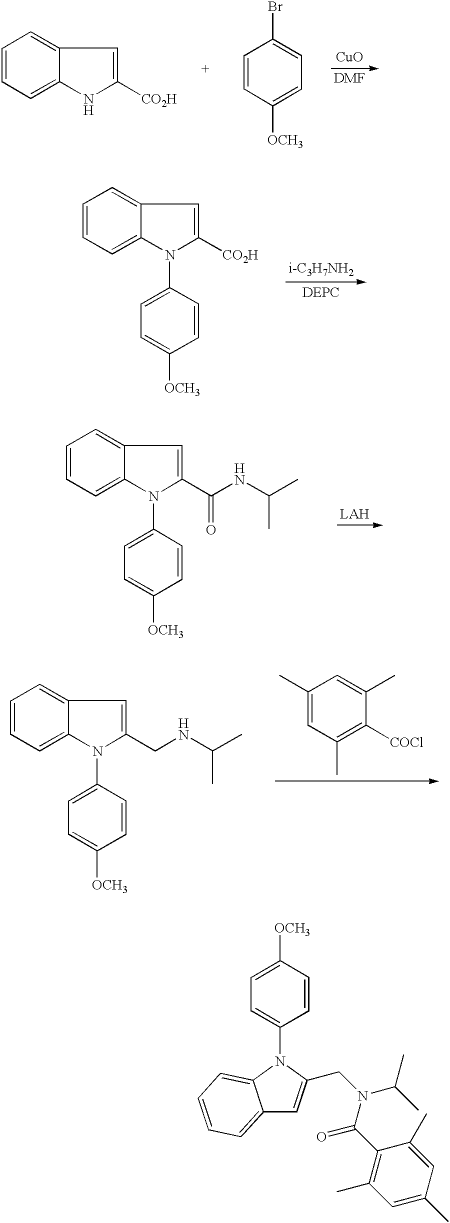

Step 1. 4-bromoanisole (40 ml, 0.32 mole) is added to a solution of indole-2-carboxylic acid (8.15 g, 50 mmol) in DMF (60 ml), followed by the addition of CuO (0.4 g, 5 mmol) and K₂CO₃ (20 g, 0.144 mole). The resulting mixture is heated to 150° C. overnight. The mixture is diluted with Et₂O, then extracted with H₂O. All the aqueous extracts are combined, and extracted with 50% EtOAc in Et₂O. The aqueous layer is then filtered, acidified to pH 1 at 0° C. with concentrated HCl. The resulting white precipitate is collected, washed with EtOAc, and dried. ¹H NMR (CDCl₃) δ3.90 (s, 3H), 7.02 (d, J=8.7 Hz, 2H), 7.08 (dd, J=8.1 and 0.6 Hz, 1H), 7.19 (ddd, J=0.9, 6.9 and 7.8 Hz, 1H), 7.26 (d, J=8.7 Hz, 2H), 7.29 (ddd, J=1.5, 7.2 and 8.4 Hz, 1H), 7.53 (d, J=0.9 Hz, 1H), 7.74 (dt, J=0.9 and 7.8 Hz, 1H). LC-MS: 268.2 (M+1).

Step 2. Et₃N (1.25 ml, 9 mmol) at 0° C. is added to a solution of the above acid (2.0 g, 7.5 mmol) in DMF (15 ml), followed by the addition of DEPC (1.52 ml, 9 mmol). The resulting solution is stirred for 20 minutes; isopropylamine (1.92 ml, 22.5 mmol) is then added. After 1 hour at room temperature, the reaction mixture is diluted with EtOAc (100 ml), and washed with water, saturated NaHCO₃, brine, dried, filtered and evaporated to give the amide as white crystalline solid. ¹H NMR (CDCl₃) δ1.08 (d, J=6.6 Hz, 6H), 3.89 (s, 3H), 4.15 (m, 1H), 5.58 (br, 1H), 7.04 (d, J=8.4 Hz, 2H), 7.10-7.30 (m, 4H), 7.30 (d, J=8.4 Hz, 2H), 7.70 (d, J=7.2 Hz, 1H). LC-MS: 309.2 (M+1).

Step 3. LAH (1N in THF, 6.8 ml, 6.8 mmol) is added to a solution of the above amide (840 mg, 2.7 mmol) in THF (4 ml). The resulting solution is heated to reflux for 20 hours, then cooled to 0° C., diluted with Et₂O, and quenched by adding water. The Et₂O layer was separated. The gel part is extracted thoroughly with Et₂O. The combined Et₂O layer is washed with water, dried, filtered and evaporated. The oil residue is then dissolved in Et₂O (8 ml) and acidified with HCl (1N). The resulting precipitate is collected, washed with water and Et₂O, and dried to give the amine in HCl salt form as a white crystalline solid. ¹H NMR (CDCl₃) (free base) δ0.94 (d, J=6.3 Hz, 6H), 2.70 (pt, J=6.3 Hz, 1H), 3.82 (s, 2H), 3.89 (s, 3H), 6.54 (s, 1H), 7.04 (d, J=9.0 Hz, 2H), 7.05-7.15 (m, 4H), 7.32 (d, J=9.0 Hz, 2H), 7.60 (m, 1H). LC-MS: 309.2 (M+1).

Step 4. Et₃N (0.25 ml, 1.75 mmol) is added to a suspension of the above salt (165 mg, 0.5 mmol) in CH₂Cl₂ (2 ml) at room temperature, followed by the addition of 2,4,6-trimethylbenzoyl chloride (0.17 ml, 1.0 mmol). The resulting mixture is stirred for 2 hours, diluted with CH₂CI₂ (4 ml), washed with water, saturated NaHCO₃, then brine, and dried, filtered and evaporated. The crude is purified by column chromatography (eluted with CH₂Cl₂ to 1% MeOH in CH₂Cl₂) to give the product as a light yellow oil. LC-MS: 441.3 (M+1). ¹H NMR (CDCl₃) shows two conformers in a ratio of about 1 to 5. Peaks derived from the major conformer: δ0.95 (d, 6H), 2.29 (s, 3H), 2.30 (s, 6H), 3.80 (m, 1H), 3.92 (s, 3H), 4.65 (s, 2H), 6.70 (s, 1H), 6.85 (s, 2H), 7.04-7.15 (m, 6H), 7.40 (d, 2H), 7.60 (m, 1H). Peaks derived from the minor conformer: 1.38 (d, 6H), 2.12 (s, 6H), 2.25 (s, 3H), 3.90 (s, 3H), 4.20 (s, 2H), the aromatic peaks overlap with the major isomer.

### Example 5

Additional compounds of the invention which may be prepared by the methods illustrated in Examples 1-4 are shown are given in Table I. All the compounds given in table I have been tested in the assay of CRF 1 receptor binding given in Example 11 and found to exhibit Ki values of 4 micromolar or less.

TABLE I

| Ex# | Structure | Name | m/e |
|---|---|---|---|
| a. | 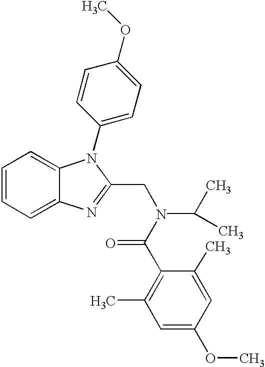 | N-Isopropyl-2,6-trimethyl-4- methoxy-N-[1-(4- methoxyphenyl)-1H- benzoimidazol-2-ylmethyl]- benzamide | 458.3 |
| b. | 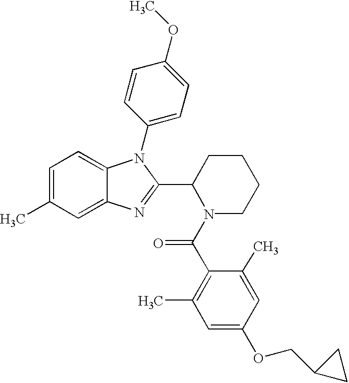 | {2-[1-(4-Methoxy-phenyl)-5- methyl-1H-benzoimidazol-2-yl]- piperidin-1-yl}-(2,6-dimethyl-4- cyclopropylmethyl-phenyl)- methanone | 524.3 |
| c. | 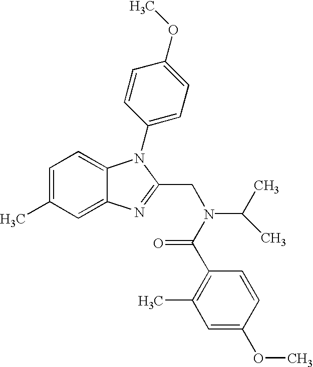 | N-Isopropyl-2,6-trimethyl-4- methoxy-N-[1-(4- methoxyphenyl)-5-methyl-1H- benzoimidazol-2-ylmethyl]- benzamide | 458.4 |
| d. | 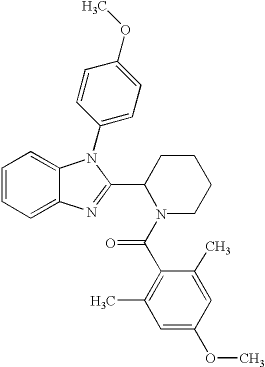 | {2-[1-(4-Methoxy-phenyl)-1H- benzoimidazol-2-yl]- piperidin- 1-yl}-(2,6-dimethyl-4- methoxy- phenyl)- methanone | 470.2 |
| e. | 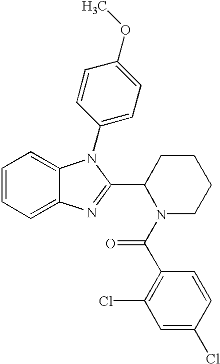 | {2-[1-(4-Methoxy-phenyl)-1H- benzoimidazol-2-yl]- piperidin- 1-yl}-(2,4-dichloro-phenyl)- methanone | 480.2 482.2 484.2 |
| f. | 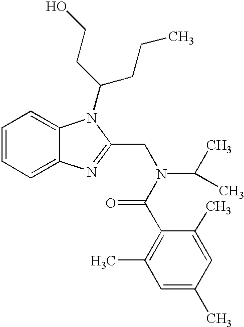 | N-Isopropyl-2,4,6-trimethyl-N- [1-(1-(3-hydroxypropyl)-butyl)- 5-trifluoromethyl-1H- benzoimidazol-2-ylmethyl]- benzamide | 436.4 |
| g. | 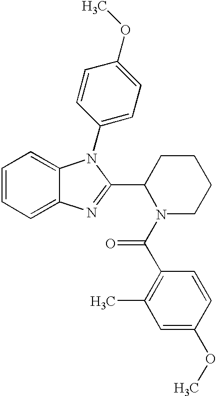 | {2-[1-(4-Methoxy-phenyl)-1H- benzoimidazol-2-yl]- piperidin- 1-yl}-(2-methyl-4-methoxy- phenyl)-methanone | 456.4 |
| h. | 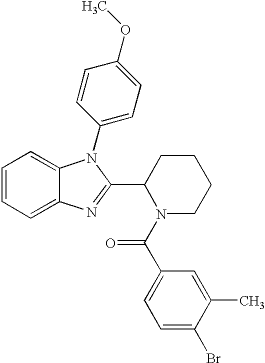 | {2-[1-(4-Methoxy-phenyl)-1H- benzoimidazol-2-yl]- piperidin- 1-yl}-(3-methyl-4-bromo- phenyl)-methanone | 504, 506 |
| i. | 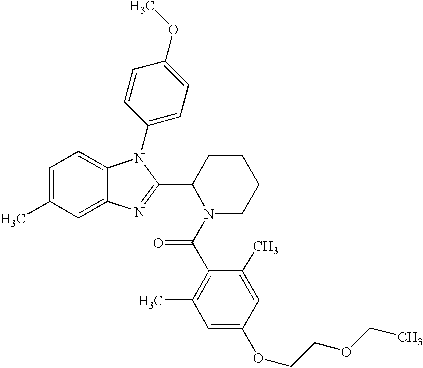 | [4-(2-Ehtoxy-ethoxy)-2,6- dimethyl-phenyl]-2{2-[1-(4- methoxy-phenyl)-5-methyl-1H- benzoimidazol-2-yl]-piperidin- 1-yl}-methanone | 542 |
| j. | 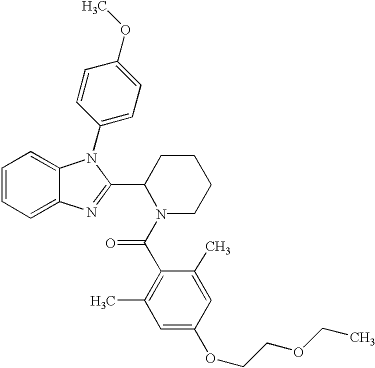 | [4-(2-Ehtoxy-ethoxy)-2,6- dimethyl-phenyl]-2{2-[1-(4- methoxy-phenyl)-1H- benzoimidazol-2-yl]-piperidin- 1-yl}-methanone | 528 |
| k. | 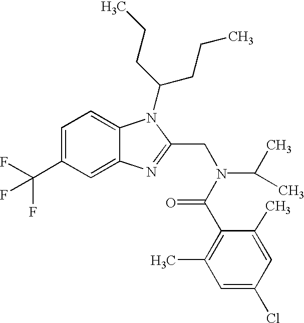 | N-Isopropyl-2,6-dimethyl-4- chloro-N-[1-(1-propyl-butyl)-5- trifluoromethyl-1H- benzoimidazol-2-ylmethyl]- benzamide | 522.3, 524.3, 526.6 |
| l. | 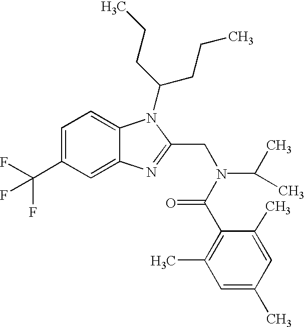 | N-Isopropyl-2,4,6-trimethyl-N- [1-(1-propyl-butyl)-5- trifluoromethyl-1H- benzoimidazol-2-ylmethyl]- benzamide | 502.4 |
| m. | 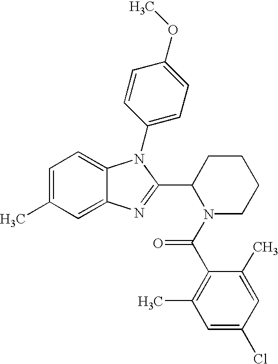 | {2-[1-(4-Methoxy-phenyl)-5- methyl-1H-benzoimidazol-2-yl]- piperidin- 1-yl}-(2,6-dimethyl-4- chloro-phenyl)-methanone | 488.8, 490.0 |
| n. | 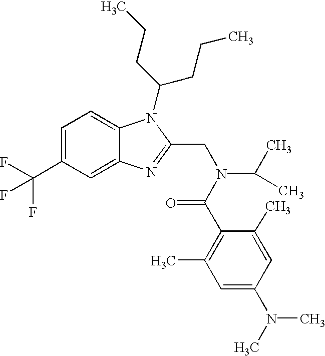 | N-Isopropyl-2,6-dimethyl-4- dimethylamino-N-[1-(1-propyl- butyl)-5-trifluoromethyl-1H- benzoimidazol-2-ylmethyl]- benzamide | 472.2 |
| o. | 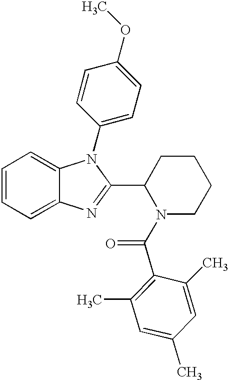 | {2-[1-(4-Methoxy-phenyl)-1H- benzoimidazol-2-yl]-piperidin- 1-yl}-(2,4,6-trimethyl-phenyl)- methanone | 454.3 |
| p. | 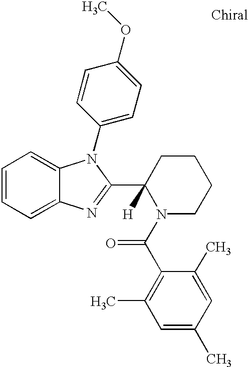 | S-{2-[1-(4-Methoxy-phenyl)- 1H-benzoimidazol-2-yl]- piperidine-1-yl}-(2,4,6-trimethyl- phenyl)-methanone | 454.3 |
| q. | 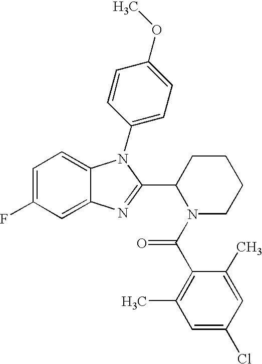 | {2-[1-(4-Methoxy-phenyl)-5- fluoro-1H-benzoimidazol-2-yl]piperidin-1-yl}-(2,6-dimethyl-4- chloro-phenyl)-methanone | 492.3, 494.3 |
| r. | 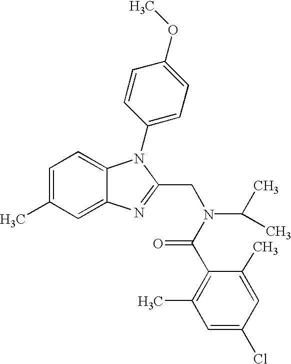 | N-Isopropyl-2,6-trimethyl-4- chloro-N-[1-(4-methoxyphenyl)- 5-methyl-1H-benzoimidazol-2- ylmethyl]-benzamide | 476.6 |
| s. | 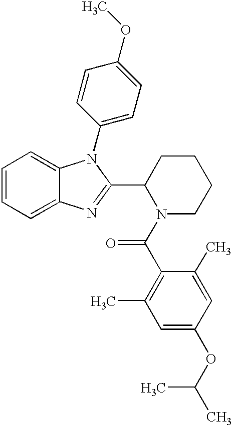 | {2-[1-(4-Methoxy-phenyl)-1H- benzoimidazol-2-yl]-piperidin- 1-yl}-(2,6-dimethyl-4- isopropoxy-phenyl)-methanone | 498.4 |
| t. | 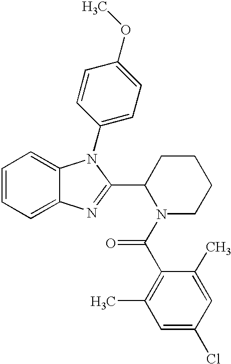 | {2-[1-(4-Methoxy-phenyl)-1H- benzoimidazol-2-yl]-piperidin- 1-yl}-(2,6-dimethyl-4-chloro- phenyl)-methanone | 474.3, 476.3 |
| u. | 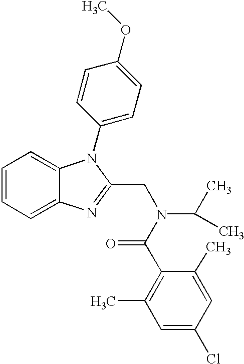 | N-Isopropyl-2,6-trimethyl-4- chloro-N-[-1(4-methoxyphenyl)- 1H-benzoimidazol-2-ylmethyl]- benzamide | 462.3, 464.3 |
| v. | 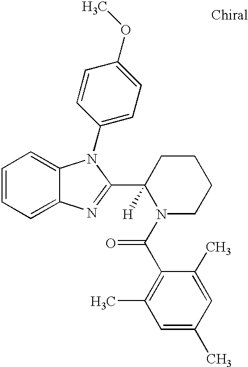 | R-{2-[1-(4-Methoxy-phenyl)- 1H-benzoimidazol-2-yl]- piperidin-1-yl}-(2,4,6-trimethyl- phenyl)-methane | 454.3 |
| w. | 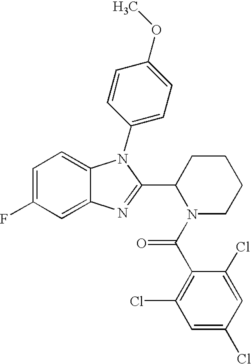 | {2-[1-(4-Methoxy-phenyl)-5- fluoro-1H-benzoimidazol-2-yl]- piperidin-1-yl}-(2,4,6-trichloro- phenyl)-methanone | 533.8 |
| x. | 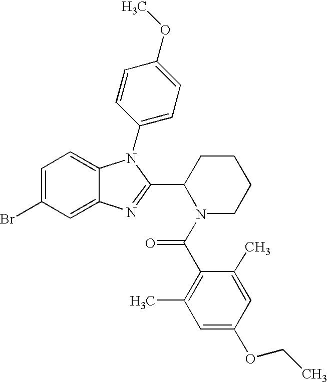 | {2-[1-(4-Methoxy-phenyl)-5- bromo-1H-benzoimidazol-2-yl]- piperidin-1-yl}-(2,6-dimethyl-4- methoxy-phenyl)-methanone | 562, 564 |
| y. | 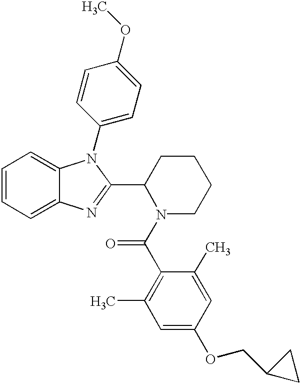 | {2-[1-(4-Methoxy-phenyl)-1H- benzoimidazol-2-yl]-piperidin- 1-yl}-(2,6-dimethyl-4- cylcopropyl-phenyl)- methanone | 510.2 |
| z. | 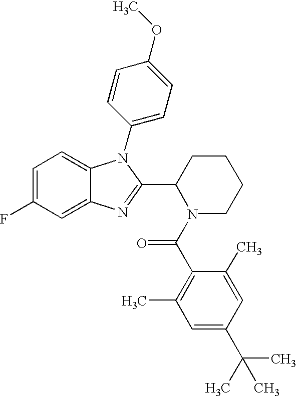 | {2-[1-(4-Methoxy-phenyl)-5- fluoro-1H-benzoimidazol-2-yl]- piperidin-1-yl}-(2,6-dimethyl-4- t-butyl-phenyl)-methanone | 515.3 |
| aa. | 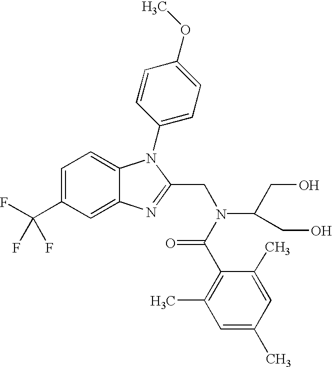 | N-2-hydroxyethylN- hydroxymethyl-2,4,6-trimethyl- N-[1-(4-methoxyphenyl)-5- methyl-1H-benzoimidazol-2- ylmethyl]-benzamide | 542.3 |
| bb. | 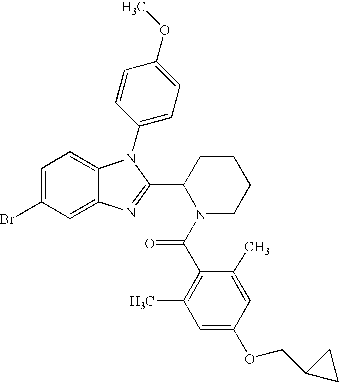 | {2-[1-(4-Methoxy-phenyl)-5- bromo-1H-benzoimidazol-2-yl]- piperidin-1-yl}-(2,6-dimethyl-4- cyclopropylmethyl-phenyl)- methanone | 543.2 |
| cc. | 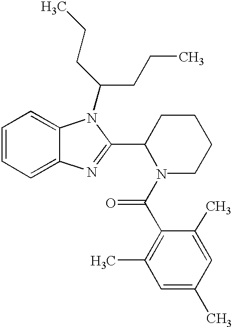 | [2-(1-Dipropylamino-1H- benzoimidazol-2-yl)-piperidin- 1-yl]-(2,4,6-trimethyl-phenyl)- methanone | 446.3 |
| dd. | 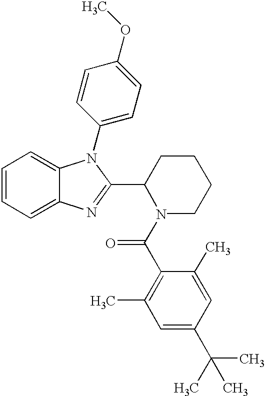 | {2-[1-(4-Methoxy-phenyl)-1H- benzoimidazol-2-yl]-piperidin- 1-yl}-(2,6-dimethyl-4-t-butyl- phenyl)-methanone | 496.3 |
| ee. | 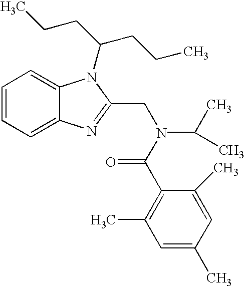 | N-Isopropyl-2,4,6-trimethyl-N- [1-(1-propyl-butyl)-1H- benzoimidazol-2-ylmethyl]- benzamide | 434.4 |
| ff. | 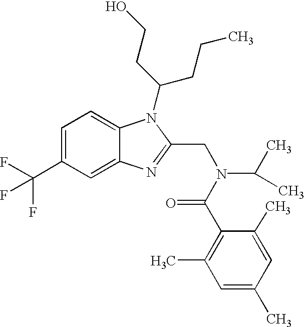 | N-{1-[1-(2-Hydroxy-ethyl)- butyl]-5-trifluoromethyl-1H- benzoimidazol-2-ylmethyl}-N- isopropyl-2, 4,6-trimethyl-benzamide | 504.4 |
| gg. | 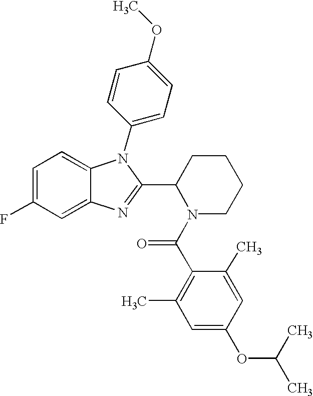 | {2-[5-Fluoro-1-(4-methoxy- phenyl)-1H-benzoimidazol-2- yl]-piperidin-1-yl}-(4- isopropoxy-2,6-dimethyl- phenyl)-methanone | 516.3 |
| hh. | 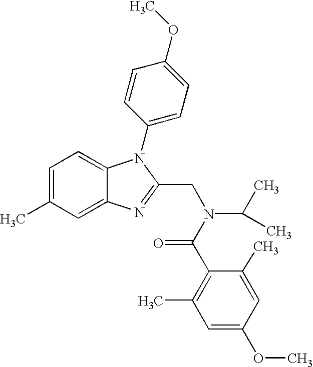 | N-Isopropyl-2,4,6-trimethyl-N- [1-(4-methoxyphenyl)-5- methyl-1H-benzoimidazol-2- ylmethyl]- benzamide | 472.2 |
| ii. | 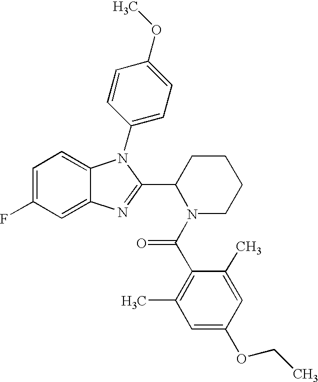 | {2-[1-(4-Methoxy-phenyl)-5- fluoro-1H-benzoimidazol-2-yl]- piperidin-1-yl}-(2,6-dimethyl-4- ethoxy-phenyl)-methanone | 502.3 |
| jj. | 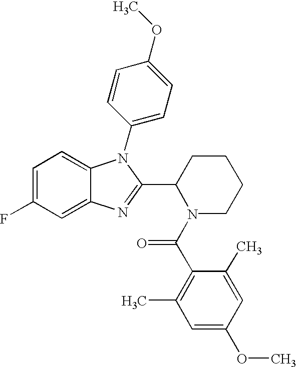 | {2-[1-(4-Methoxy-phenyl)-5- fluoro-1H-benzoimidazol-2-yl]- piperidin-1-yl}-(2,6-dimethyl-4- methoxy-phenyl)-methanone | 488.3 |
| kk. | 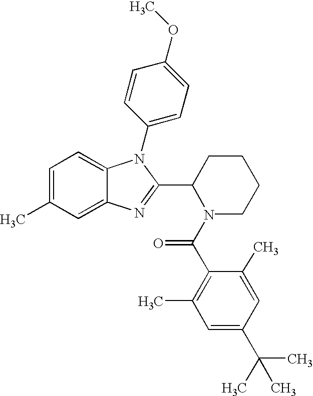 | {2-[1-(4-Methoxy-phenyl)-5- methyl-1H-benzoimidazol-2-yl]- piperidin-1-yl}-(2,6-dimethyl-4- t-butyl-phenyl)-methanone | 512.7 |
| ll. | 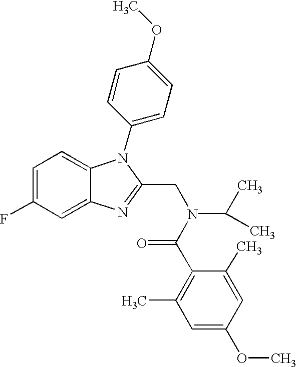 | N-Isopropyl-2,6-dimethyl-4- methoxy-N-[1-(4- methoxyphenyl)-5-fluoro-1H- benzoimidazol-2-ylmethyl]- benzamide | 476.2 |
| mm. | 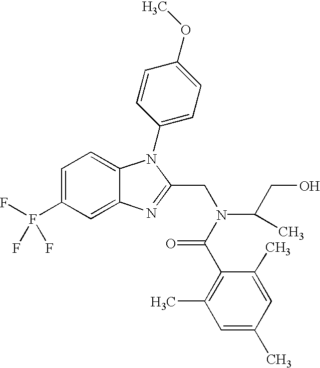 | N-(2-hydroxy-1-methyl-ethyl)-2,6-dimethyl-4-methoxy-N-]1- (4-methoxyphenyl)-5- trifluoromethyl-1H- benzoimidazol-2-ylmethyl]- benzamide | 526.5 |
| nn. | 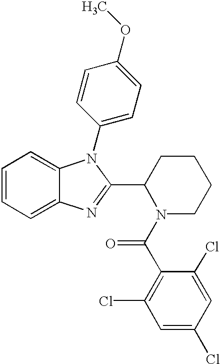 | {2-[1-(4-Methoxy-phenyl)-1H- benzoimidazol-2-yl]-piperidin- 1-yl}-(2,4,6-trichloro-phenyl)- methanone | 513.9, 515.9, 517.7, 521.9 |
| oo. | 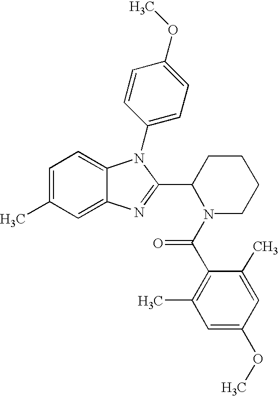 | {2-[1-(4-Methoxy-phenyl)-5- methyl-1H-benzoimidazol-2-yl]- piperidin- 1-yl}-(2,6-dimethyl-4- methoxy-phenyl)-methanone | 484.1 |
| pp. | 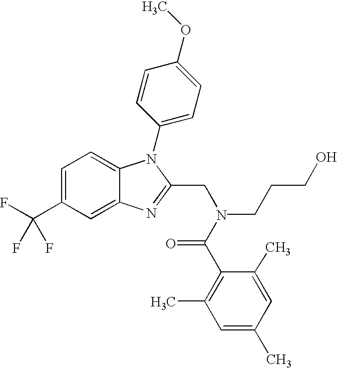 | N-3-hydroxy-propyl-2,4,6- trimethyl-N-[1-(4- methoxyphenyl)-5- trifluoromethyl-1H- benzoimidazol-2-ylmethyl]- benzamide | 526.3 |
| qq. | 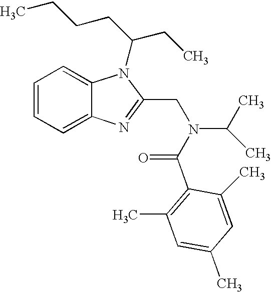 | N-Isopropyl-2,4,6-trimethyl-N- [1-(1-ethyl-pentyl)-1H- benzoimidazol-2-ylmethyl]- benzamide | 434.6 |
| rr. | 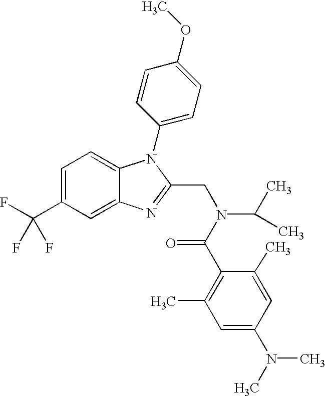 | N-Isopropyl-2,6-dimethyl-4- dimethylamino-N-[1-(4- methoxyphenyl)-5- trifluoromethyl-1H- benzoimidazol-2-ylmethyl]- benzamide | 539.4 |
| ss. | 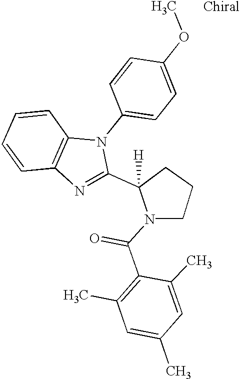 | {2-[1-(4-Methoxy-phenyl)-1H- benzoimidazol-2-yl]-piperidin- 1-yl}-(2,4,6-trimethyl-phenyl)- methanone | 440.3 |
| tt. | 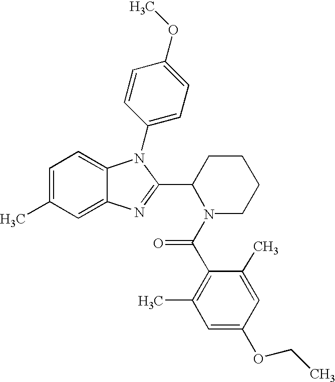 | {2-[1-(4-Methoxy-phenyl)-5- methyl-1H-benzoimidazol-2-yl]- piperidin-1-yl}-(2,6-dimethyl-4- ethoxy-phenyl)-methanone | 498.3 |
| uu. | 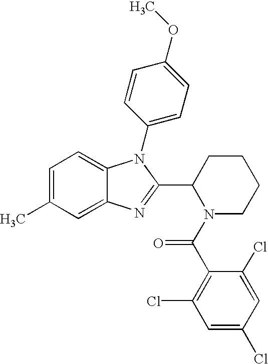 | {2-[1-(4-Methoxy-phenyl)-5- methyl-1H-benzoimidazol-2-yl]- piperidin-1-yl}-(2,4,6-trichloro- phenyl)-methanone | 527.6 529.6, 531.6, 533.6 |
| vv. | 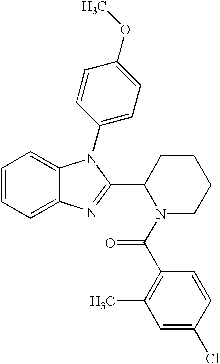 | {2-[1-(4-Methoxy-phenyl)-1H- benzoimidazol-2-yl]-piperidin- 1-yl}-(2-methyl-4-chloro- phenyl)-methanone | 460.3, 462.3 |
| ww. | 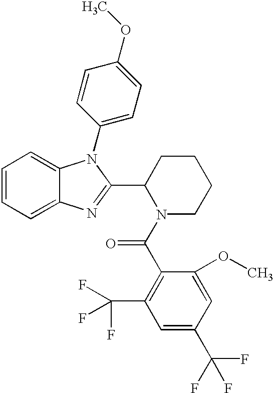 | {2-[1-(4-Methoxy-phenyl)-1H- benzoimidazol-2-yl]-piperidin- 1-yl}-(2,4-bis-trifluoromethyl-6- methoxy-phenyl)-methanone | 578 |
| xx. | 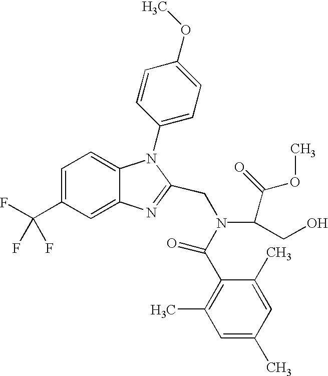 | 2-{Benzoyl-[1-(4-methoxy- phenyl)-5-trifluoromethyl-1H- benzoimidazol-2-ylmethyl]- amino}-3-hydroxy-propionic acid methyl ester | 570.3 |
| yy. | 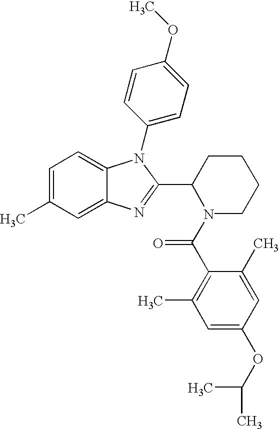 | {2-[1-(4-Methoxy-phenyl)-5- methyl-1H-benzoimidazol-2-yl]- piperidin-1-yl}-(2,6-dimethyl-4- isopropoxy-phenyl)- methanone | 512.1 |
| zz. | 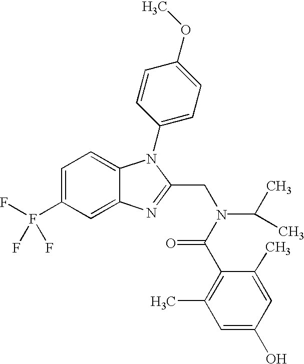 | 4-Hydroxy-N-isopropyl-N-[1-(4- methoxy-phenyl)-5- trifluoromethyl-1H- benzoimidazol-2-ylmethyl]-2,6- dimethyl-benzamide | 512.3 |
| aaa. | 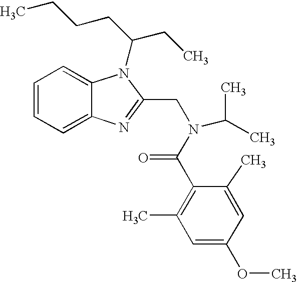 | N-Isopropyl-2,6-dimethyl-4- methoxy-N-[1-(1-ethyl-pentyl)- 1H-benzoimidazol-2-ylmethyl]- benzamide | 450.3 |
| bbb. | 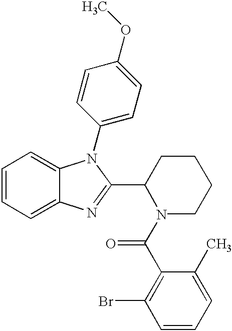 | {2-[1-(4-Methoxy-phenyl)-1H- benzoimidazol-2-yl]-piperidin- 1-yl}-(2-bromo-6-methyl- phenyl)-methanone | 504, 506 |
| ccc. | 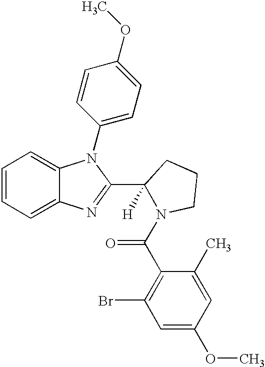 | (4-Methoxy-2,6-dimethyl- phenyl)-{2-[1-(4-methoxy- phenyl)-1H-benzoimidazol-2- yl]-pyrrolidin-1-yl}-methanone | 456.3 |
| ddd. | 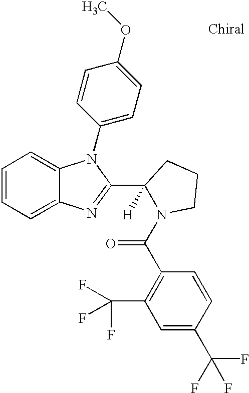 | (4-Methoxy-2,4-bis- trifluoromethy-phenyl)-{2-[1-(4- methoxy-phenyl)-1H benzoimidazol-2-yl]-pyrrolidin- 1-yl}-methanone | 434.2 |
| eee. | 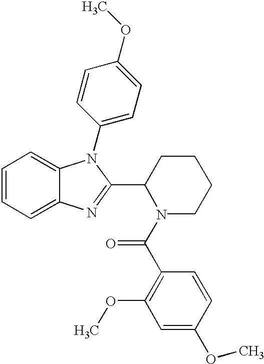 | {2-[1-(4-Methoxy-phenyl)-1H- benzoimidazol-2-yl]-piperidin- 1-yl}-(2,6-dimethyl-phenyl)- methanone | 472.1 |
| fff. | 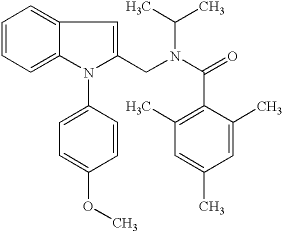 | N-Isopropyl-2,4,6-trimethyl-N- [1-(4-methoxyphenyl)-5-fluoro- 1H-benzoimidazol-2-ylmethyl]- benzamide | 441.3 |
| ggg. | 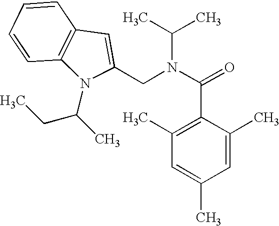 | N-(1-sec-Butyl-1H-indol-2- ylmethyl)-N-isopropyl-2,4,6- trimethyl-benzamide | 391.4 |
| hhh. | 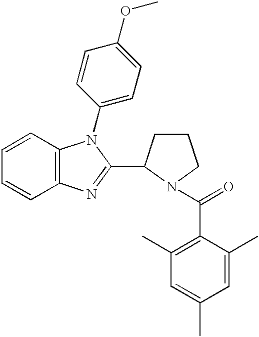 | {2-[1-(4-Methoxy-phenyl)-1H- benzoimidazol-2-yl]-pyrrolidin- 1-yl}-(2,4,6-trimethyl-phenyl)- methanone | |

### Example 6

Preparation of {2-[5-Bromo-1-(4-methoxy-phenyl)-1H-benzoimidazol-2-yl]-piperidin-1-yl}-(2,4,6-trimethyl-phenyl)-methanone

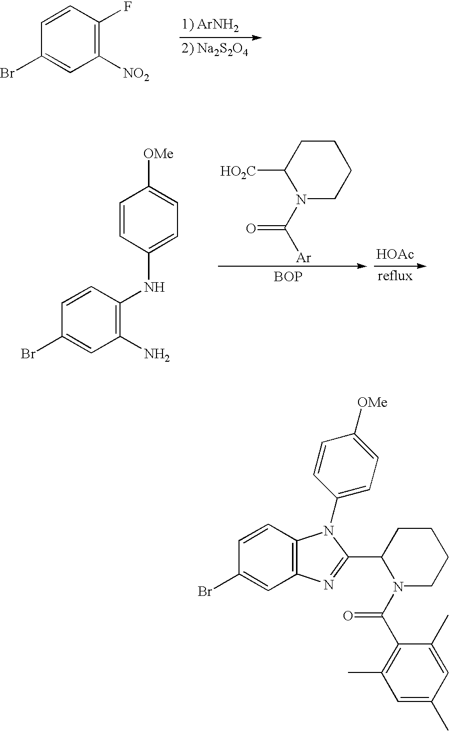

Step 1. Potassium fluoride (2 g) is added to a solution of 5-Bromo-2-fluoronitrobenzene (5 g, 22.7 mmol) and p-anisidine (4.2 g, 1.5 eq) in ethanol (50 mL). The mixture is sealed and heated at 160° C. for 1 hour. After the reaction is complete, the mixture is allowed to cool to room temperature, and is then taken up into dichloromethane and washed with 0.1N HCl. The organic layer is separated, dried, concentrated and the residue recrystallized from ethanol to give a red crystalline solid.

Step 2. The nitroaniline (2.0 g) is dissolved in THF (25 mL). Concentrated ammonium hydroxide (8 mL), sodium dithionite (5 g), and water (8 mL) is added to the nitroaniline solution. The mixture is stirred at room temperature for 3 hours, concentrated in vacuo to remove THF, diluted with aqueous sodium hydroxide and vigrously stirred at 0° C. for 30 minutes. The resulting purple powder is filtered and air dried to give crude diamine.

Step 3. A solution of the diamine (0.91 g), acid (0.93 g, 1.1 eq), and BOP (benzotriazol-1-yloxytris(dimethylamino)phosphonium hexafluorophosphate, 1.5 g) in N,N-dimethylformamide (5 mL) containing triethylamine (0.75 mL) is stirred at room temperature overnight. After that, additional BOP (0.3 g) is added, and the mixture is briefly heated to 80° C. The cooled reaction mixture is taken up in ethyl acetate, washed successively with aqueous sodium bicarbonate and 1N HCl before being dried and concentrated. The residue is dissolved in glacial acetic acid (20 mL) and heated at reflux overnight. Concentration followed by normal aqueous work-up and silica gel column chromatography gives the desired benzimidazole, {2-[5-Bromo-1-(4-methoxy-phenyl)-1H-benzoimidazol-2-yl]-piperidin-1-yl}-(2,4,6-trimethyl-phenyl)-methanone (Ex #6). MS, m/e=534. 1H nmr (400 MHz, CDCl3), d 1.4-2.0 (m, 6H), 2.1 (s, 3H), 2.2 (s, 3H), 2.25 (s, 3H), 3.4 (br d, 1H), 3.9 (s, 3H), 4.2 (t, 1H), 6.1 (br s, 1H), 6.8 (s, 2H), 7.0 (d, 1H), 7.1 (d, 2H), 7.3 (d, 1H), 7.5 (br, 1H), 7.9 (s, 1H).

| Ex# | Structure | Name | NMR | MS m/e |
|---|---|---|---|---|
| 6a | 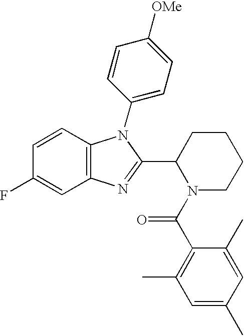 | {2-[5-Fluoro-1-(4-methoxy- phenyl)-1H-benzoimidazol-2-yl]- piperidin-1-yl}-(2,4,6-trimethyl- phenyl)-methanone | 1.4-2.0 (m, 6H), 2.1 (s, 3H), 2,2 (s, 3H), 2.25 (s, 3H), 3.4 (br d, 1H), 3.9 (s, 3H), 4.2 (t, 1H), 6.1 (br s, 1H), 6.8 (s, 2H), 7.0 (m, 2H), 7.1 (app d, 2H), 7.4 (d, 2H), 7.5 (br, 1H) | 472 |
| 6b | 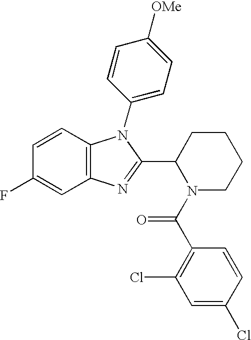 | {2-[5-Fluoro-1-(4-methoxy- phenyl)-1H-benzoimidazol-2-yl]- piperidin-1-yl}-(2,4-dichloro- phenyl)-methanone | 1.5-2 (m,6H + 6 H′), 3.3 (br d, 2H), 3.9 (s, sH + 3H′), 4.0 (br t, 2H′), 4.4 (m, 1H), 4.8 (m, 1H′), 6.0 (br s, 1H′), 6.1 (br s, 1H), 6.9-7.5 (m, 10H + 10 H′) [H and H′denote protons of major and minor rotamer, respectively] | 498 |
| 6c | 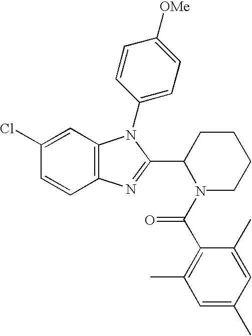 | {2-[5-Chloro-1-(4-methoxy- phenyl)-1H-benzoimidazol-2-yl]- piperidin-1-yl}-(2,4,6-trimethyl- phenyl)-methanone | 1.4-2.0 (m, 6H), 2.1 (s, 3H), 2.2 (s, 3H), 2.3 (s, 3H), 3.4 (br d, 1H), 3.9 (s, 3H), 4.2 (t, 1H), 6.2 (br s, 1H), 6.8 (s, 2H), 7.05 (s, 1H), 7.1 (d, 2H), 7.2 (d, 2H), 7.5 (d, 1H), 7.6 (d, 1H) | 488 |
| 6d | 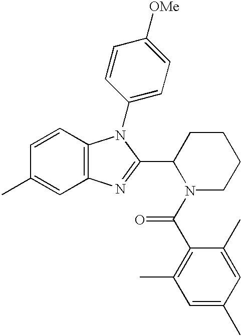 | {2-[5-Methyl-1-(4-methoxy- phenyl)-1H-benzoimidazol-2-yl]- piperidin-1-yl}-(2,4,6-trimethyl- phenyl)-methanone | 1.4-2.0 (m. 6H), 2.1 (s, 3H), 2.2 (s, 3H), 2.3 (s, 3H), 2.5 (s, 3H), 3.4 (br d, 1H), 3.9 (s, 3H), 4,2 (t, 1H), 6.2 (br s, 1H), 6.8 (s, 2H), 6.9-7.1 (m, 4H), 7.5 (br, 2H), 7.6 (s, 1H) | 468 |
| 6e | 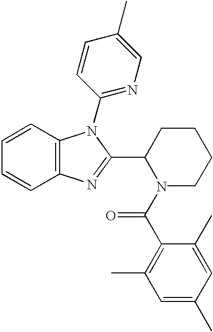 | {2-[1-(5-methyl-pyrid-2-yl)-1H- benzoimidazol-2-yl]-piperidin-1- yl}(2,4,6-trimethyl-phenyl)- methanone | 1.4-2.0 (m, 6H), 2.0 (s, 3H), 2.2 (s, 3H), 2.25 (s, 3H), 2.5 (s, 3H), 3.4 (br d, 1H), 4.2 (t, 1H), 6.4 (d, 1H), 6.8 (d, 2H), 7.2-7.4 (m, 3H), 7.6 (d, 1H), 7.8 (m, 2H), 8.6 (s, 1H) | 439 |
| 6f | 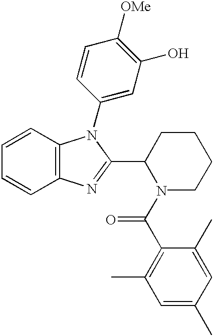 | {2-[1-(3-hydroxy-4-methoxy- phenyl)-1H-benzoimidazol-2-yl]- piperidin-1-yl}-(2,4,6-trimethyl- phenyl)-methanone | 1.4-2.0 (m, 6H), 2.0 (s, 3H), 2.2 (s, 3H), 2.25 (s, 3H), 3.4 (br d, 1H), 4.0 (s, 3H), 4.2 (br, 1H), 6.3 (d, 1H), 6.8 (d, 2H), 7.0-7.3 (m, 6H), 7.8 (d, 2H) | 470 |
| 6g | 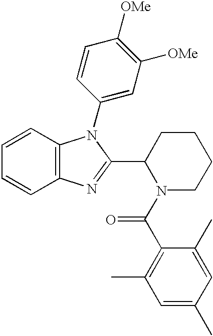 | {2-[1-(3,4-dimethoxy-phenyl)- 1H-benzoimidazol-2-yl]- piperidin-1-yl}-(2,4,6-trimethyl- phenyl)-methanone | 1.4-2.0 (m, 6H), 2.0 (s, 3H), 2.2 (s, 3H), 2.25 (s, 3H), 3.4 (br d, 1H), 3.9 (br s, 3H), 4.0 (s, 3H), 4.3 (br t, 1H), 6.3 (br s, 1H), 6.8 (d, 2H), 7.0-7.3 (m, 6H), 7.8 (d, 2H) | 484 |
| 6h | 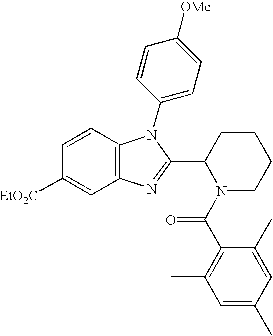 | 1-(4-Methoxy-phenyl)-2-[1- (2,4,6-trimethyl-benzoyl)- piperidin-2-yl]-1H- benzoimidazole-5-carboxylic acid ethyl ester | 1.4 (t, 3H), 1.4-2.0 )m, 6H), 2.0 (s, 3H), 2.2 (s, 3H), 2.25 (s, 3H), 3.9 (s, 3H), 4.3 (t, 1H), 4.4 (m, 2H), 6.2 (br s, 1H), 6.9 (d, 2H), 7.0 7.1 (m, 3H), 7.3 (br, 1H), 7.6 (br, 1H), 8.0 (d, 1H), 8.5 (s, 1H) | |
| 6i | 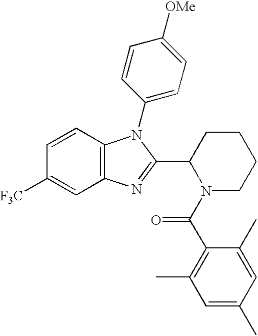 | {2-[5-Trifluoromethyl-1-(4- methoxy-phenyl)-1H- benzoimidazol-2-yl]-piperidin-1- yl}-(2,4,6-trimethyl-phenyl)- methanone | 1.4-2.0 (m, 6H), 2.1 (s, 3H), 2.2 (s, 3H), 2.3 (s, 3H), 3.4 (br, 1H), 3.9 (s, 3H), 4.2 (br, 1H), 6.2 (br s, 1H), 6.8 (s, 2H), 7.2 (m, 2H), 7.5 (br 1H, 7.7 (v br, 1H), 8.1 (s, 1H) | 522 |
| 6j | 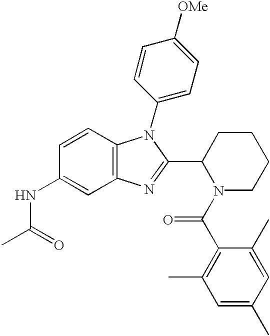 | N-}1-(4-Methoxy-phenyl)-2-[1- (2,4,6-trimethyl-benzoyl)- piperidin-2-yl]-1H- benzoimidazol-5-yl}- acetamide | 1.4-2.0 (m, 6H), 2.1 (s, 3H), 2.2 (s, 3H), 2.25 (s, 3H), 2.3 (s, 3H), 3.4 (br, 1H), 3.9 (s, 3H), 4.2 (br, 1H), 6.2 (br s, 1H), 6.8 (s, 2H), 7.0 (d, 1H), 7.1 (d, 2H), 7.4 (d, 1H), 7.2-7.5 (v. br, 2H), 7.9 (s, 1H) | 511 |

### Example 7

Preparation of {2-[5-Substituted-1-(4-methoxy-phenyl)-1H-benzoimidazol-2-yl]-piperidin-1-yl}-(2,4,6-trimethyl-phenyl)-methanone compounds

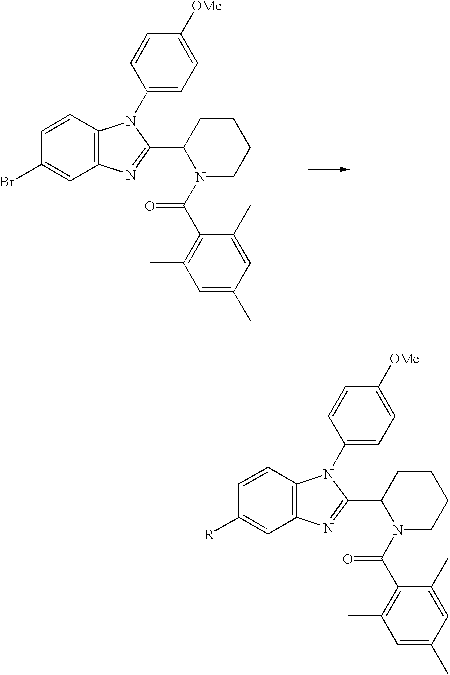

Example 7a [R=Et]; Dichloronickel(II) tetrakis(triphenylphosphine) (5 mg) is added to a solution of the boromobenzimidazole (50 mg) in THF (1 mL), followed by the addition of ethylmagnesium bromide (3M in ether, 0.10 mL). The mixture is stirred at room temperature for 1 hour. Normal aqueous workup and column chromatography give the desired ethylbenzimidazole, {2-[5-ethyl-1-(4-methoxy-phenyl)-1H-benzoimidazol-2-yl]-piperidin-1-yl}-(2,4,6-trimethyl-phenyl)-methanone. MS, m/e=482. 1H nmr (400 MHz, CDCl3), d 1.3 (t, 3H), d 1.4-2.0 (m, 6H), 2.1 (s, 3H), 2.2 (s, 3H), 2.25 (s, 3H), 2.8 (q, 2H), 3.4 (br d, 1H), 3.9 (s, 3H), 4.2 (t, 1H), 6.2 (br s, 1H), 6.8 (d, 2H), 7.0 (d, 1H), 7.1 (d, 211), 7.3 (d, 1H), 7.5 (br, 1H), 7.9 (s,

Example 7b [R=Ph]; May be prepared in the same manner as ethyl derivative. Product name {2-[5-phenyl-1-(4-methoxy-phenyl)-1H-benzoimidazol-2-yl]-piperidin-1-yl}-(2,4,6-trimethyl-phenyl)-methanone: MS, m/e=530. 1H nmr (400 MHz, CDCl3), d 1.4-2.0 (m, 6H), 2.1 (s, 3H), 2.2 (s, 3H), 2.25 (s, 3H), 3.4 (br d, 1H), 3.9 (s, 3H), 4.2 (t, 1H), 6.2 (br s, 1H, 6.8 (d, 2H), 7.1 (m, 3H), 7.4 (t, 1H), 7.5 (m, 4H), 7.7 (d, 2H), 8.0 (s, 1H).

Example 7c [R=CN]; A mixture of the bromobenzimidazole (300 mg) and copper cyanide (150 mg) in N,N-dimethylformamide is heated at 200° C. for 2.5 h and then at 160° C. overnight. The mixture is allowed to cool and poured into ammonium hydroxide - ammonium chloride and vigorously stirred for 30 minutes. Extraction and column chromatography gives the desired nitrile, {2-[5-cyano-1-(4-methoxy-phenyl)-1H-benzoimidazol-2-yl]-piperidin-1-yl}-(2,4,6-trimethyl-phenyl)-methanone: MS, m/e=479. 1H nmr (400 MHz, CDCl3), d 1.4-2.0 (m, 6H), 2.1 (s, 3H), 2.2 (s, 3H), 2.25 (s, 3H), 3.4 (br d, 1H), 3.9 (s, 3H), 4.2 (t, 1H), 6.2 (br s, 1H), 6.8 (d, 2H), 7.2 (m, 3H), 7.5 (d, 1H), 7.7 (br, 1H), 8.1 (s, 1H).

Example 7d [R=C(O)NMe₂]; Aminocarboxybenzimidazole (R=CONH2, 25 mg), a side-product of the cyanation reaction described above, is dissolved in DMF (1 mL). Sodium hydride (60% in mineral oil, 10 mg) and iodomethane (10 uL) are added successively. After 2 hours at room temperature, normal aqueous workup and chromatography on silica gel SPE (solid phase extraction) gives the desired product, 1-(4-Methoxy-phenyl)-2-[1-(2,4,6-trimethyl-benzoyl)-piperidin-2-yl]-1H-benzoimidazole-5-carboxylic acid dimethylamide: 1H nmr (400 MHz, CDCl3), d 1.4-2.0 (m, 6H), 2.1 (s, 3H), 2.2 (s, 3H), 2.25 (s, 3H), 3.1 (br s, 6H), 3.4 (br d, 1H), 3.9 (s, 3H), 4.2 (t, 1H), 6.2 (br s, 1H), 6.8 (d, 2H), 7.2 (m, 3H), 7.5 (d, 1H), 7.7 (br, 1H), 8.1 (s, 1H).

Example 7e [R=5-tetrazole]; A mixture of cyanobenzimidazole (above, 18 mg), sodium azide (100 mg), and ammonium chloride (100 mg) in N-methylpyrrolidinone (0.5 mL) are heated at 100° C. for 2 days and left to stand for 3 days more. The mixture is diluted with water and extracted twice with ethyl acetate. Combined extracts are dried, concentrated, and chromatographed to give the tetrazole product, {2-[5-(5-tetrazolyl)-1-(4-methoxy-phenyl)-1H-benzoimidazol-2-yl]-piperidin-1-yl}-(2,4,6-trimethyl-phenyl)-methanone: MS, m/e=522.

Example 7f [R=3-pyridyl]; The bromobenzimidazole (50 mg), 3-pyridineboronic acid (25 mg), tris(dibenzylideneacetone)dipalladium (5 mg), and bis(diphenylphosphino)ferrocene (dppf, 5 mg) are placed in a flask. The reaction vessel is charged with argon, and ethylene glycol dimethyl ether (2 mL) is added, followed by the addition of IM sodium carbonate (1 mL). The mixture is stirred overnight at 70° C. before normal aqueous workup and silica gel column chromatography, which yields the desired 5-(3-pyridyl)benzimidazole, {2-[5-(3-pyridyl)-1-(4-methoxy-phenyl)-1H-benzoimidazol-2-yl]-piperidin-1-yl}-(2,4,6-trimethyl-phenyl)-methanone. MS, m/e=531. 1H nmr (400 MHz, CDCl3), d 1.4-2.0 (m, 6H), 2.1 (s, 3H), 2.2 (s, 3H), 2.25 (s, 3H), 2.8 (q, 2H), 3.4 (br d, 1H), 3.9 (s, 3H), 4.2 (t, 1H), 6.2 (d, 1H). 6.8 (d, 2H), 7.0-7.2 (m, 3H), 7.4 (m, 3H), 7.6 (br, 1H), 8.0 (m, 2H), 8.6 (d, 1H), 8.9 (s, 1H).

Example 7g [R=4-pyridyl]; May be prepared in the same manner as 3-pyridyl derivative. Product name: {2-[5-(4-pyridyl)-1-(4-methoxy-phenyl)-1H-benzoimidazol-2-yl]-piperidin-1-yl}-(2,4,6-trimethyl-phenyl)-methanone MS, m/e=531. 1H nmr (400 MHz, CDCl3), d 1.4-2.0 (m, 6H), 2.1 (s, 3H), 2.2 (s, 3H), 2.25 (s, 311), 2.8 (q, 2H), 3.4 (br d, 1H), 3.9 (s, 3H), 4.2 (t, 1H), 6.2 (d, 1H). 6.8 (d, 2H), 7.0 - 7.2 (m, 3H), 7.5 (d, 1H), 7.6 (d, 2H), 7.6 (br, 1H), 8.1 (s, 1H), 8.6 (d, 2H).

### Example 8

Preparation of {2-[1-(4-Methoxy-phenyl)-5-morpholin-4-ylmethyl-1H-benzoimidazol-2-yl]-piperidin-1-yl}-(2,4,6-trimethyl-phenyl)-methanone

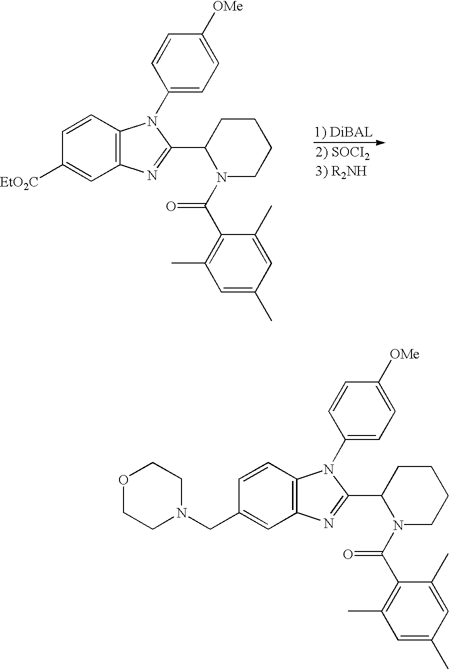

Step 1. Diisobutylaluminum hydride (20% in toluene, 2.0 mL)is added dropwise to a solution of the ethyl ester (440 mg) in dichloromethane (10 mL) at −78° C. After the addition, the mixture is further stirred at −78° C. for 1 hour and then allowed to warm to 0° C. over a 30 minute period before being quenched by 1N HCl. The mixture is vigorously stirred for 30 minutes and extracted twice with dichloromethane. The organic layers are combined and washed with aqueous sodium bicarbonate, dried, filtered, and concentrated. Column chromatography of the residue results in isolation of the alcohol.

Step 2. Thionyl chloride (0.2 mL) is added to a solution of the alcohol (410 mg) in dichloromethane (4 mL). The mixture is stirred at room temperature for 1 hour, and then concentrated in vacuo to give the crude chloride as its hydrochloride salt.

Step 3. The chloromethylbenzimidazole (HCl salt, 25 mg) is dissolved in acetonitrile (1 mL) and morpholine (30 uL) is added. After 3 hours at room temperature, the mixture is directly chromatographed on silica gel to give the desired product (Ex #7). MS, m/e=553. 1H nmr (400 MHz, CDCl3), d 1.4-2.0 (m, 6H), 2.1 (s, 3H), 2.2 (s, 3H), 2.25 (s, 3H), 2.5 (br s, 4H), 3.4 (br d, 1H), 3.6 (br s, 2H), 3.7 (br s, 4H), 3.9 (s, 3H), 4.2 (t, 1H), 6.2 (d, 1H). 6.8 (d, 2H), 7.0 (d, 1H), 7.1 (d, 2H), 7.2 (d, 1H), 7.2-7.7 (v br, 2H), 7.7 (s, 1H).

| Ex # | Example | Name | 1H nmr | MS, m/e |
|---|---|---|---|---|
| 7a | 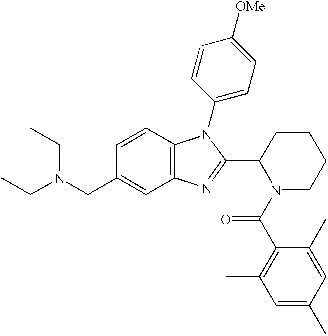 | {-[5-Diethylamino methyl- 1-(4-methoxy-phenyl)- 1H-benzo imidazol-2-yl]- piperidin-1-yl}-(2,4,6- trimethyl-phenyl)- methanone | | 539 |
| 7b | 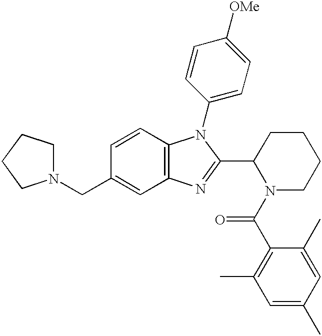 | {2-[1-(4-Methoxy-phenyl)- 5-pyrrolidin-1-ylmethyl- 1H-benzoimidazol-2-yl]- piperidin-1-yl}-(2,4,6- trimethyl-phenyl)- methanone | d 1.4-2.0 (m, 10H), 2.1 (s, 3H), 2.2 (s, 3H), 2.25 (s, 3H), 2.6 (br s, 4H), 3.4 (br d, 1H), 3.8 (br q, 2H), 3.9 (s, 3H), 4.2 (t, 1H), 6.2 (d, 1H). 6.8 (d, 2H), 7.0 (d, 1H), 7.1 (d, 2H), 7.2 (d, 1H, 7.2-7.7 (v br, 2H), 7.7 (s, 1H) | 537 |
| 7c | 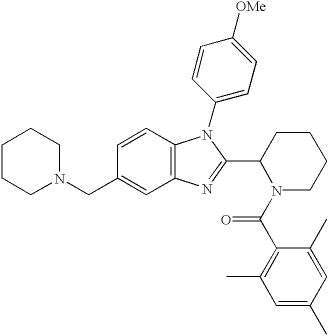 | {2-[1-(4-Methoxy-phenyl)- 5-piperidin-1-ylmethyl-1H- benzoimidazol-2-yl]- piperidin-1-yl}-(2,4,6- trimethyl-phenyl)- methanone | | 551 |
| 7e | 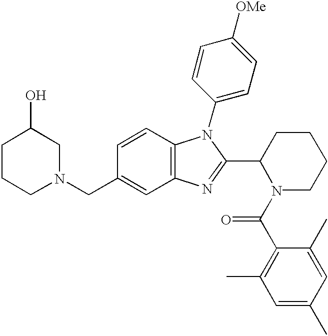 | {2-[1-(4-Methoxy-phenyl)- 5-(3-hydroxy-piperidin-1- ylmethyl-1H-benzo imidazol-2-yl]-piperidin-1- yl}-(2,4,6-trimethyl- phenyl)- methanone | | 567, 466 |
| 7f | 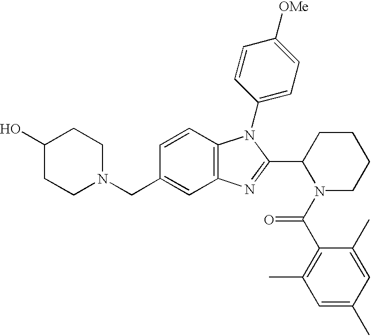 | {2-[1-(4-Methoxy-phenyl)- 5-(4-hydroxy-piperidin-1- ylmethyl-1H-benzo imidazol-2-yl]-piperidin-1- yl}-(2,4,6-trimethyl- phenyl)- methanone | | 567, 466 |
| 7g | 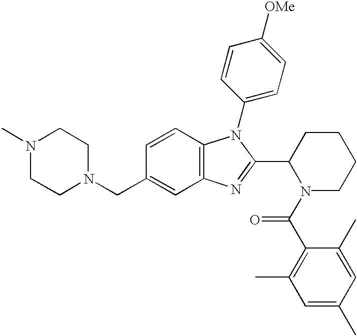 | {2-[1-(4-Methoxy-phenyl)- 5-(4-methyl-piperazin-1- ylmethyl-1H-benzo imidazol-2-yl]-piperidin-1- yl}-(2,4,6-trimethyl- phenyl)- methanone | | 566, 466 |

### Example 9

Preparation of additional {2-[5-Substituted-1-(4-methoxy-phenyl)-1H-benzoimidazol-2-yl]-piperidin-1-yl}-(2,4,6-trimethyl-phenyl)-methanone compounds

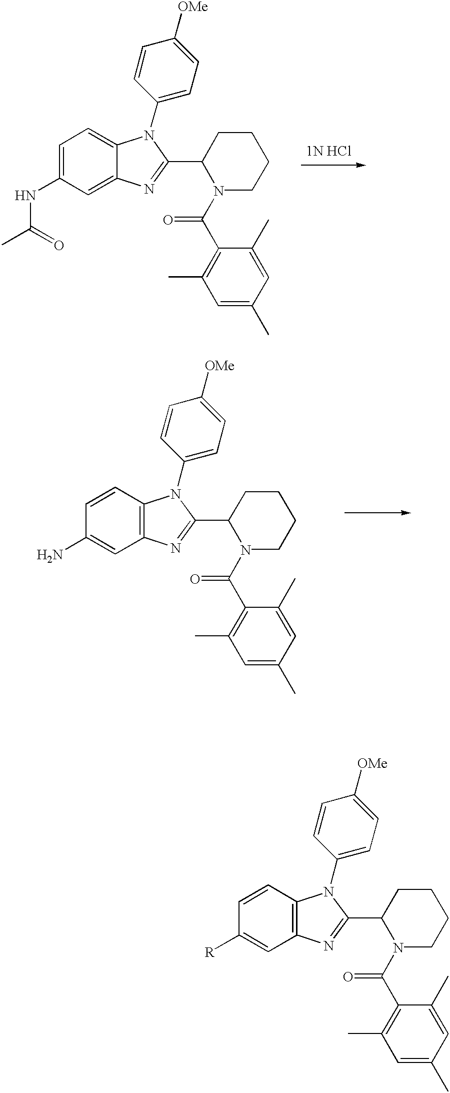

Example 9a [R=NH₂]; N-{1-(4-Methoxy-phenyl)-2-[1-(2,4,6-trimethyl-benzoyl)-piperidin-2-yl]-1H-benzoimidazol-5-yl}-acetamide is prepared by the method given in Example 7, Step 1, using 2-Fluoro-5-acetamidobezimdiazole as a starting material. 1N HCl (6 mL) is added to a solution of the acetamidobenzimidazole (800 mg) in methanol (6 mL) and the mixture is heated to reflux overnight. Normal aqueous workup and chromatography gives {2-[5-Amino-1-(4-methoxy-phenyl)-1H-benzoimidazol-2-yl]-piperidin-1-yl}-(2,4,6-trimethyl-phenyl)-methanone as the product. MS, m/e=469. ¹H nmr (400 MHz, CDCl3), d 1.4-2.0 (m, 6H), 2.1 (s, 3H), 2.2 (s, 3H), 2.3 (s, 3H), 3.4 (br, 1H), 3.6 (v. br, 2H), 3.9 (s, 3H), 4.2 (t, 1H), 6.2 (d, 1H), 6.6 (d, 2H), 6.8 (d, 2H), 6.9 (d, 2H), 7.1 (app s, 3H), 7.4 (v. br, 2H).

Example 9b [R=NMe₂]; Paraformaldehyde (100 mg) and sodium cyanoborohydride (100 mg) are added to a solution of aminobenzimidazole (100 mg) in acetic acid (1 mL). The mixture is heated at 80° C. for 30 minutes, diluted with water, basified with ammonium hydroxide, and extracted with ether. Organics were combined, dried, and concentrated and the residue chromatographed on silica gel to give the dimethylamino-benzimicazole, {2-[5-Dimethylamino-1-(4-methoxy-phenyl)-1H-benzoimidazol-2-yl]-piperidin-1-yl}-(2,4,6-trimethyl-phenyl)-methanone. MS, m/e=, m/e=497. ¹H nmr (400 MHz, CDCl3), d 1.4-2.0 (m, 6H), 2.1 (s, 3H), 2.2 (s, 3H), 2.3 (s, 3H), 3.0 (s, 6H), 3.4 (br, 1H), 3.9 (s, 3H), 4.2 (t, 1H), 6.2 (d, 1H), 6.8 (m, 4H), 7.0 (d, 1H), 7.1 (d, 2H), 7.2 (s, 1H), 7.4 (v. br, 2H).

Example 9c [R=NEt₂]; Sodium triacetoxyborohydride (50 mg) and acetaldehyde (20 uL) are added to a solution of aminobenzimidazole (20 mg) in dichloromethane (1 mL). The mixture is stirred at room temperature for 30 minutes, diluted with ethyl acetate, washed with sodium bicarbonate, dried, concentrated, and purified by silica gel SPE to give the desired diethylamiino-benzimidazole, {2-[5-Dimethylamino-1-(4-methoxy-phenyl)-1H-benzoimidazol-2-yl]-piperidin-1-yl}-(2,4,6-trimethyl-phenyl)-methanone. MS, m/e=525. 1H nmr (400 MHz, CDCl3), d 1.2 (t, 6H), 1.4-2.0 (m, 6H), 2.1 (s, 3H), 2.2 (s, 3H), 2.3 (s, 3H), 3.4 (br s, 5H), 3.9 (s, 3H), 4.2 (t, 1H), 6.2 (d, 1H), 6.8 (m, 4H), 7.0 (d, 1H), 7.1 (d, 2H), 7.4 (v. br, 2H).

Example 9d [R=1-piperazine]; The aminobenzimidazole prepared as described in Example 10a is converted to N,N-bis(chloroethyl)aminobenzimidazole, using chloroacetaldehyde, via the same reductive amination procedures as above. The bis(chloroethyl)aminobenzimidazole (18 mg) is dissolved in methanol (1 mL) and concentrated ammonium hydroxide (1 mL) added. The mixture is sealed and heated at 80° C. overnight. Evaporation of volatiles is followed by extractive workup and column chromatography to give the desired product {2-[1-(4-Methoxy-phenyl)-5-piperidin-1-ylmethyl-1H-benzoimidazol-2-yl]-piperidin-1-yl}-(2,4,6-trimethyl-phenyl)-methanone: MS, m/e=538.

Example 9e [R=NHSO₂Me]; Triethylamine (30 uL) and methansulfonyl chloride (20 uL) are added to a solution of aminobenzimidazole (20 mg) in dichloromethane (1 mL). After 1 hour at room temperature, the mixture is diluted with ethyl acetae, washed with sodium bicarbonate, dried, and concentrated to give a mixture of mono- and bis-sulfonylamide. The crude product is dissolved in methanol (1 mL) and treated with lOM sodium hydroxide (30 uL) at room temperature overnight. The mixture is then concentrated and diluted with aqueous ammonium chloride. The resulting precipitate is collected by filtration and air-dried to give the desired N-{1-(4-Methoxy-phenyl)-2-[1-(2,4,6-trimethyl-benzoyl)-piperidin-2-yl]-1H-benzoimidazol-5-yl}-methanesulfonamide MS, m/e=547.

### Example 10

Preparation of N-Isopropyl-N-[1-(4-methoxy-phenyl)-5-trifluoromethyl-1H-benzoimidazol-2-ylmethyl]-2,4,6-trimethyl-benzamide

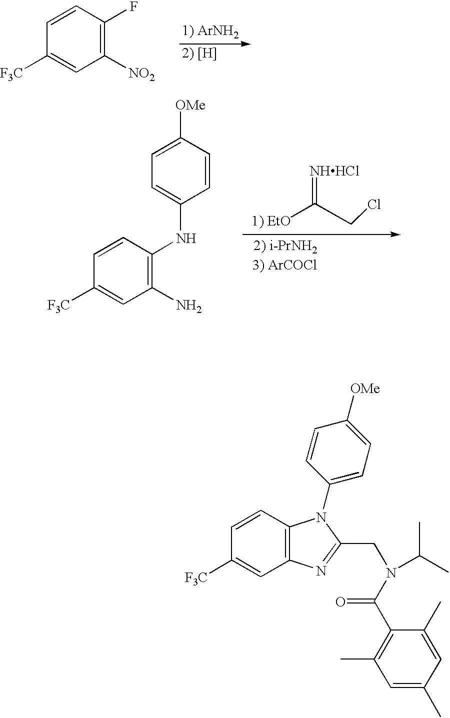

Step 1. A mixture of 2-fluoro-5-trifluoromethylnitrobenzene (10.4 g, 50 mmol) and anisidine (0.70 g) is heated at 120° C. under nitrogen in the presence of potassium fluoride (3 g) for 4 hours. After cooling, the mixture is dissolved in dichloromethane, washed with 0.1N HCl, dried, concentrated, and recrystallized in ethanol to give the nitroaniline (13 g) as red crystals.

Step 2. Ammonium hydroxide (25 mL), sodium dithionite (13 g), and water (20 mL) are added to a solution of the nitroaniline (5.0 g) in THF (75 mL). The mixture is stirred vigorously at room temperature for 4 hours until the red color disappears, concentrated in vacuo to remove THF, diluted with 1N sodium hydroxide, and extracted with dichloromethane. Drying and evaporation gave 4.5 g of crude diamine.

Step 3. A mixture of the diamine (1.0 g) and ethyl chloroacetimidate hydrochloride (1.0 g) in ethanol (10 mL) is stirred overnight at room temperature and then diluted with water. The resulting precipitate is collected by filtration, air-dried, and redissolved in N,N-dimethylformamide (5 mL). Isopropylamine (2 mL) is added to the solution and the mixture is stirred at room temperature overnight. Usual aqueous workup and evaporation gives 1.1 g of crude amine which is sufficiently pure to be used in the next step.

Step 4. Triethylamine (0.15 mL) and 2,4,6-trichloromethylbenzoyl chloride (70 uL) are added to a solution of the amine (120 mg) in dichloromethane (4 mL). The mixture is stirred at room temperature overnight. Aqueous workup and column chromatography gives the desired product as a white foam, which is converted to the hydrochloride salt and recrystallized from ethyl acetate to give the product as a white powder. MS, m/e=510. 1H nmr (400 MHz, CDCl3), d 1.4 (d, 6H), 2.2 (s, 6H), 2.25 (s, 3H), 3.8 (br, 1H), 4.0 (s, 3H), 5.0 (br s, 2H), 6.8 (s, 2H), 7.2 (m, 3H), 7.5 (d, 1H), 7.8 (br d, 2H), 8.4 (s, 1H).

| EX# | Structure | Name | 1H nmr | MS, m/e = |
|---|---|---|---|---|
| 10a | 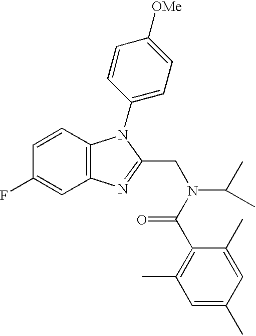 | N-[5-Fluoro-1-(4-methoxy- phenyl)-1H-benzoimidazol-2- ylmethyl]-N-isopropyl-2,4,6- trimethyl-benzamide | 1.2 (d, 6H), 2.3 (s, 9H), 3.9 (m, 1H), 3.95 (s, 3H), 4.6 (s, 2H), 6.8 (s, 2H), 7.0 (m, 2H), 7.1 (s, 2H), 7.4 (d, 1H), 7.5 (s, 2H) | 460 |
| 10b | 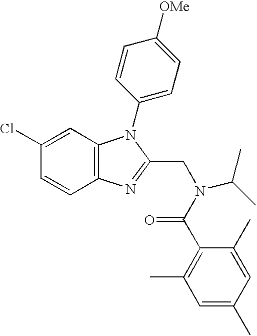 | N-[6-Chloro-1-(4-methoxy- phenyl)-1H-benzoimidazol-2- ylmethyl]-N-isopropyl-2,4,6- trimethyl-benzamide | 1.2 (d, 6H), 2.2 (s, 9H), 3.8 (m, 1H), 3.9 (s, 3H), 4.6 (s, 2H), 6.8 (s, 2H), 7.05 (s, 1H), 7.1 (d, 2H), 7.2 (d, 1H), 7.5 (d, 2H), 7.6 (d, 1H) | 476 |
| 10c | 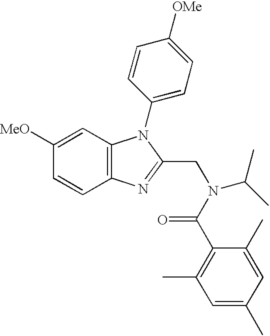 | N-[6-Methoxy-1-(4-methoxy- phenyl)-1H-benzoimidazol-2- ylmethyl]-N-isopropyl-2,4,6- trimethyl-benzamide | 1.2 (d, 6H), 1.4 (d, 6H′), 2.1 (s, 6H′), 2.3 (s, 9H + 3H′), 3.8 (s, 3H + 3H′), 3.8 (s, 3H + 3H′), 3.8(m, 1H), 3.9 (s, 3H +3H′), 4.2 (s, 2H′), 4.6 (s, 2H), 4.9 (m, 1H′), 6.4 (s, 1H′), 6.6 (s, 1H), 6.7 (s, 2H′), 6.8 (s, 2H), 6.9 (m, 1H +1H′), 7.1 (d, 2H +2H′), 7.5 (d, 2H + | |
| # 1H′) [H and H′ denote protons of major and minor rotamer, respectively] | 472 | | | |
| 10d | 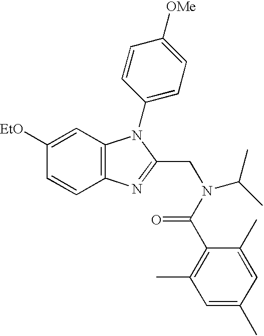 | N-[6-Ethoxy-1-(4-methoxy- phenyl)-1H-benzoimidazol-2- ylmethyl]-N-isopropyl-2,4,6- trimethyl-benzamide | 1.2 (d, 6H), 1.4 (t, 3H), 2.2 (s, 9H), 3.8 (m, 1H), 3.9 (s, 3H), 4.0 (q, 2H), 4.6 (s, 2H), 6.6 (s, 1H), 6.8 (s, 2H), 6.85 (d, 1H), 7.1 (d, 2H), 7.5 (d, 2H), 7.6 (d, 1H) | 486 |
| 10e | 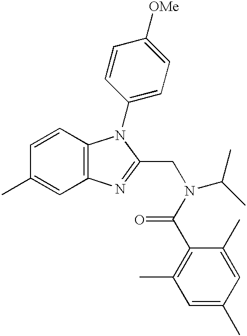 | N-[5-Methyl-1-(4-methoxy- phenyl)-1H-benzoimidazol-2- ylmethyl]-N-isopropyl-2,4,6- trimethyl-benzamide | 1.2 (d, 6H), 1.3 (d 6H′), 2.1 (s, 6H′), 2.2 (s, 9H), 2.3 (s, 3H′), 2.5 (s, 3H), 3.8 (m, 1H + 1H′), 3.9 (2s, 3H +3H′), 4.2 (s, 2H′), 4.7 (s, 2H), 6.6-7.1 (m, 7H + 7H′), 7.5 (d, 2H), 7.55 (d, 2H′) [H and H′ denote protons of major and minor rotamer, respectively] | 456 |
| 10f | 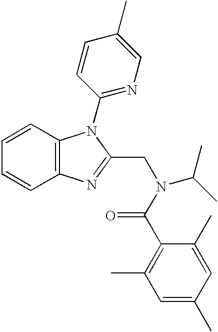 | N-[1-(4-methoxy-phenyl)-1H- benzoimidazol-2-ylmethyl]-N- isopropyl-2,4,6-trimethyl- benzamide | 1.2 (d, 6H), 2.2 (2s, 9H), 2.5 (s, 3H), 3.8 (m, 1H), 5.0 (s, 2H), 6.8 (s, 2H), 7.2 (m, 2H), 7.4 (d, 2H), 7.6 (d, 2H), 7.8 (m, 2H), 8.5 (s, 1H) | 427 |
| 10g | 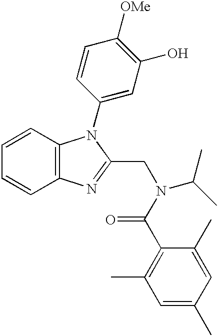 | N-[1-(3-hydroxy-4-methoxy)- phenyl)-1H-benzoimidazol-2- ylmethyl]-N-isopropyl-2,4,6- trimethyl-benzamide | 1.2 (s, 6H), 2.3 (2s, 9H), 3.8 (m, 1H), 4.0 (s, 3H), 4.7 (s, 2H), 6.0 (s, 1H), 6.8 (s, 2H), 7.0-7.3 (m, 5H), 7.6 (d, 1H) | 458 |
| 10h | 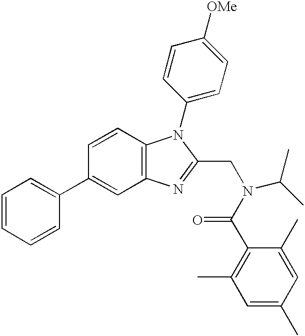 | N-[5-Phenyl-1-(4-methoxy- phenyl)-1H-benzoimidazol-2- ylmethyl]-N-isopropyl-2,4,6- trimethyl-benzamide | 1.2 (6, 6H), 2.3 (2s, 9H), 3.9 (m, 1H), 3.95 (s, 3H), 4.6 (s, 2H), 6.8 (s, 2H), 7.1 (m, 4H), 7.4-7.6 (m, 5H), 7.6 (d, 2H), 8.0 (s, 1H) | 518 |
| 10i | 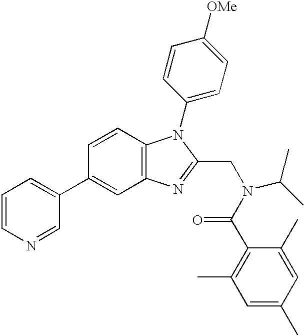 | N-[5-(Pyrid-3-yl)-1-(4-methoxy- phenyl)-1H-benzoimidazol-2- ylmethyl-N-N-isopropyl-2,4,6- trimethyl-benzamide | 1.2 (d, 6H), 2.3 (2s, 9H), 3.9 (m, 1H), 3.95 (s, 3H), 4.6 (s, 2H), 6.8 (s, 2H), 7.15 (d, 2H), 7.2 (d, 1H), 7.4 (m, 2H), 7.6 (d, 2H), 8.0 (s, 2H), 8.6 (s, 1H), 8.9 (s, 1H) | 519 |
| 10j | 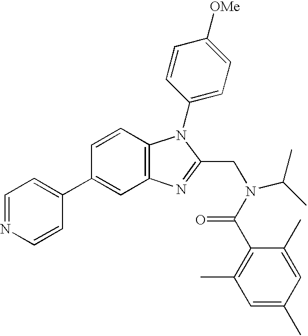 | N-[5-(Pyrid-3-yl)-1-(4-methoxy- phenyl)-1H-benzoimidazol-2- ylmethyl-N-N-isopropyl-2,4,6- trimethyl-benzamide | 1.2 (d, 6H), 2.3 (2s, 9H), 3.9 (m, 1H), 3.95 (s, 3H), 4.6 (s, 2H), 6.8 (s, 2H), 7.15 (d, 2H), 7.2 (d, 1H), 7.4-7.7 (m, 6H), 8.0 (s, 1H), 8.6 (d, 2H) | 519 |
| 10k | 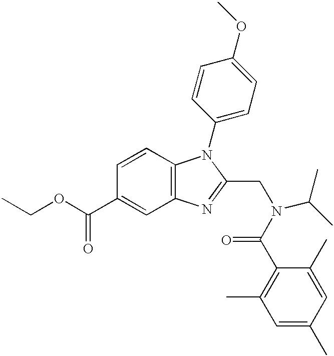 | 2-}[Isopropyl-(2,4,6-trimethyl- benzoyl)-amino]methyl}-1-(4- methoxy-phenyl)-1H- benzoimidazole-5-carboxylic acid ethyl ester | 1.2 9d, 6H), 1.4(t, 3H), 2.3 (2s, 9H), 3.9 (m, 1H), 3.95 (s, 3H), 4.4 (q, 2H), 4.6 (s, 2H), 6.8 (s, 2H), 7.1 (m, 2H), 7.5 (d, 2H), 7.9 (d, 1H), 8.4 (s, 1H) | |
| 10l | 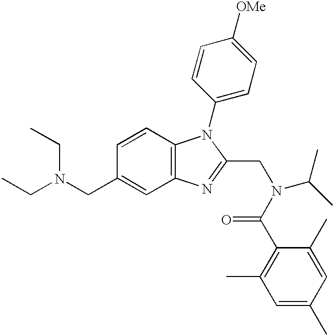 | N-[5-(Diethylaminomethyl)-1-(4- methoxy-phenyl)-1H- benzoimidazol-2-ylmethyl]-N- isopropyl-2,4,6-trimethyl- benzamide | 527 | |
| 10m | 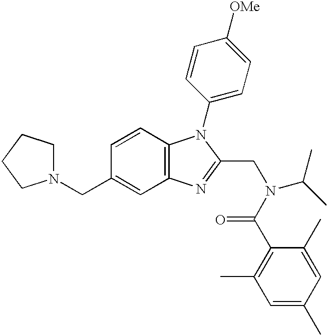 | N-Isopropyl-N-[1-(4-methoxy- phenyl)-5-pyrrolidin-1-ylmethyl- 1H-benzoimidazol-2-ylmethyl]- 2,4,6-trimethyl-benzamide | | 525 |
| 10m | 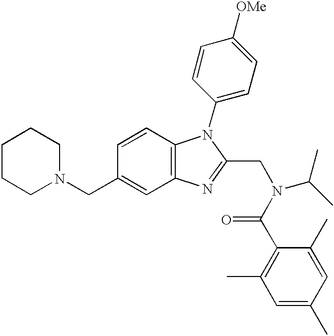 | N-Isopropyl-N-[1-(4-methoxy- phenyl)-5-pyrrolidin-1-ylmethyl- 1H-benzoimidazol-2-ylmethyl]- 2,4,6-trimethyl-benzamide | | 539 |
| 10n | 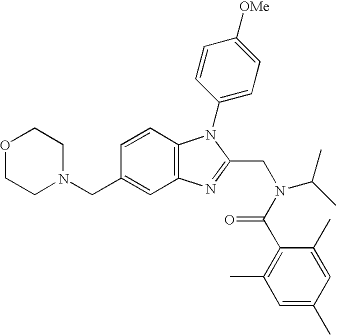 | N-Isopropyl-N-[1-(4-methoxy- phenyl)-5-morpholin-4- ylmethyl-1H-benzoimidazol-2- ylmethyl]-2,4,6-trimethyl- benzamide | | 541 |
| 10o | 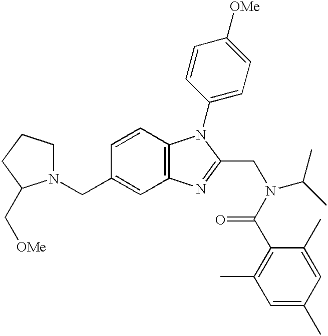 | N-Isopropyl-N-[5-(2- methoxymethyl-pyrrolidin-1- ylmethyl-1-(4-methoxy- phenyl)-1H-benzoimidazol-2- ylmethyl]-2,4,6-trimethyl- benzamide | | 69, 454 |
| 10p | 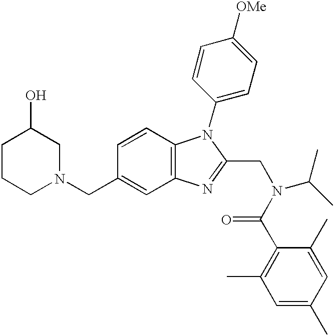 | N-Isopropyl-N-[1-(4-methoxy- phenyl)-5-(3-hydroxy-piperidin- 1-ylmethyl-1H-benzoimidazol- 2-ylmethyl]-2,4,6-trimethyl- benzamide | | 55, 454 |
| 10q | 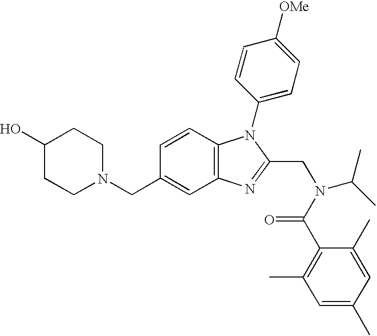 | N-Isopropyl-N-[1-(4-methoxy- phenyl)-5-(4-hydroxy-piperidin- 1-ylmethyl-1H-benzoimidazol- 2-ylmethyl]-2,4,6-trimethyl- benzamide | | 55, 454 |
| 10r | 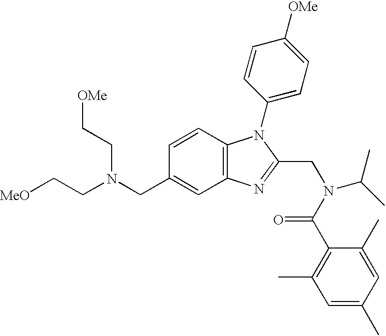 | N-[5-{[Bis-(2-methoxy-ethyl)- amino]-methyl}-1-(4-methoxy- phenyl)-1H-benzoimidazol-2- ylmethyl]-N-isopropyl-2,4,6- trimethyl-benzamide | 587, 454 | |
| 10s | 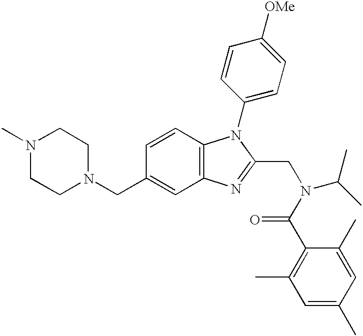 | N-Isopropyl-N-[1-(4-methoxy- phenyl)-5-(4-methyl-piperidin-1- ylmethyl-1H-benzoimidazol-2- ylmethyl]-2,4,6-trimethyl- benzamide | | 454 |
| 10t | 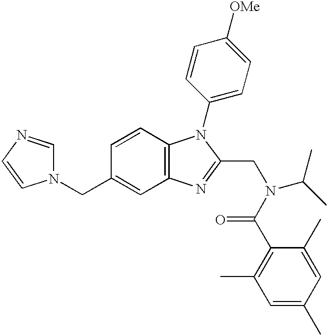 | N-Isopropyl-N-[1-(4-methoxy- phenyl)-5-(imidazol-1-ylmethyl)- 1H-benzoimidazol-2-ylmethyl]- 2,4,6-trimethyl-benzamide | | 522, 454 |
| 10u | 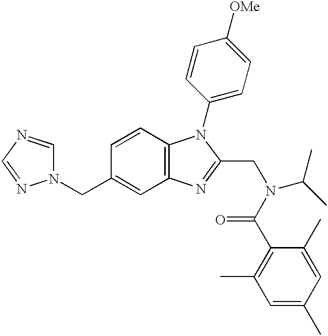 | N-Isopropyl-N-[1-(4-methoxy- phenyl)-5-[1,2,4]triazol-1- ylmethyl-1H-benzoimidazol-2- ylmethyl]-2,4,6-trimethyl- benzamide | | 523, 490 |
| 10v | 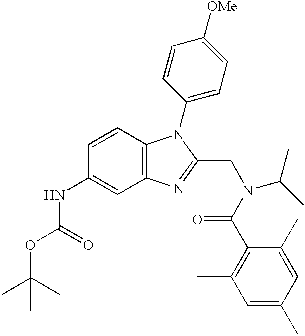 | [2-{[Isopropyl-(2,4,6-trimethyl- benzoyl)-amino]-methyl}-1-(4- methoxy-phenyl)-1H- benzoimidazol-5-yl]-carbamic acid tert-butyl ester | 1.2 (d, 6H), 1.5 (s, 9H), 2.3 (s, 9H), 3.8 (m, 1H), 3.9 (s, 3H), 4.6 (s, 2H), 6.5 (br s, 1H), 6.8 (s, 2H), 7.0 (d, 1H), 7.1 (d, 2H), 7.2 (br d, 1H), 7.5 (d, 2H), 7.8 (s, 1H) | 557, 501 |
| 10w | 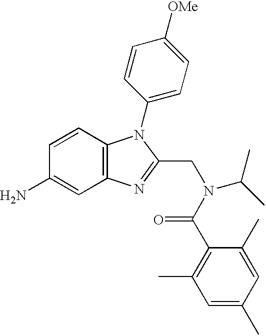 | N-[5-Amino-1-(4-methoxy- phenyl)-1H-benzoimidazol-2- ylmethyl]-N-isopropyl-2,4,6- trimethyl-benzamide | 1.2 (d, 6H), 2.3 (2s, 9H), 3.8 (m, 1H), 3.9 (s, 3H), 4.6 (s, 2H), 6.6 (d, 1H), 6.8 (s, 2H), 6.9 (d, 1H), 7.1 (m, 3H, 7.5 (d, 2H) | 457 |
| 10x | 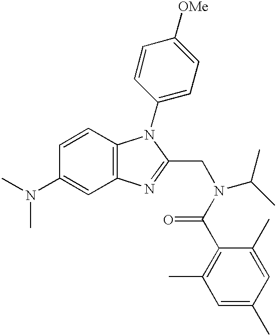 | N-[5-Dimethylamino-1-(4- methoxy-phenyl)-1H- benzoimidazol-2-ylmethyl]-N- isopropyl-2,4,6-trimethyl- benzamide | 1.2 (d, 6H), 2.3 (2s, 9H), 3.0 (s, 6H), 3.8 (m, 1H), 3.9 (s, 3H), 4.6 (s, 2H), 6.6 (s, 1H), 6.8 (s, 2H), 7.0 (d, 1H), 7.1 (d, 2H), 7.2 (s, 1H), 7.5 (d, 2H) | |
| 10y | 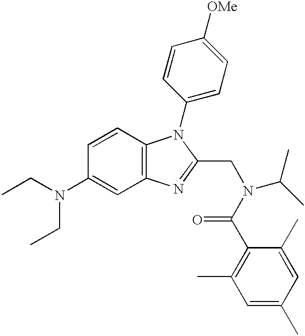 | N-[5-Dimethylamino-1-(4- methoxy-phenyl)-1H- benzoimidazol-2-ylmethyl]-N- isopropyl-2,4,6-trimethyl- benzamide | 1.2 (m, 12H), 2.3 (2s, 9H), 3.4 (m, 4H), 3.8 (m, 1H), 3.9 (s, 3H), 4.6 (s, 2H), 6.6 (d, 1H), 6.8 (s, 2H), 7.0 (d, 1H), 7.1 (d, 2H), 7.5 (d, 2H) | 513 |
| 10z | 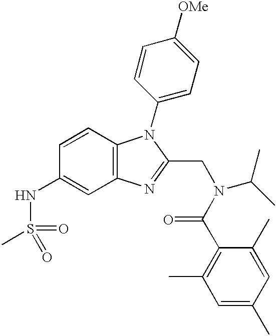 | N-Isopropyl-N-[5- methanesulfonylamino-1-(4- methoxy-phenyl)-1H- benzoimidazol-2-ylmethyl]- 2,4,6-trimethyl-benzamide | 1.2 (d, 6H), 2.3 (s, 3H), 2.4 (s, 6H), 2.8 (s, 3H), 3.8 )m + s, 4H), 4.6 (s, 2H), 6.7 (s, 1H), 6.8 (s, 2H), 6.9 (m. 2H), 7.1 (m, 3H), 7.1 (s, 1H), 7.5 (d, 2H) | 535 |

### Example 11

### Assay for CRF Receptor Binding Activity

As discussed above, the following assay is defined herein as a standard in vitro CRF receptor binding assay.

The pharmaceutical utility of compounds of this invention is indicated by the following assay for CRF1 receptor activity. The CRF receptor binding is performed using a modified version of the assay described by Grigoriadis and De Souza (Methods in Neurosciences, Vol. 5, 1991). IMR-32 human neuroblastoma cells, a cell-line that naturally expresses the CRF1 receptor, are grown in IMR-32 Medium, which consists of EMEM w/Earle's BSS (JRH Biosciences, Cat#51411) plus, as supplements, 2 mM L-Glutamine, 10% Fetal Bovine Serum, 25 mM HEPES (pH 7.2), 1 mM Sodium Pyruvate and Non-Essential Amino Acids (JRH Biosciences, Cat#58572). The cells are grown to confluence and split three times (all splits and harvest are carried out using NO-ZYME—JRH Biosciences, Cat#59226). The cells are first split 1:2, incubated for 3 days and split 1:3, and finally incubated for 4 days and split 1:5. The cells are then incubated for an additional 4 days before being differentiated by treatment with 5-bromo-2′deoxyuridine (BrdU, Sigma, Cat#B9285). The medium is replaced every 3-4 days with IMR-32 medium w/2.5uM BrdU and the cells are harvested after 10 days of BrdU treatment and washed with calcium and magnesium-free PBS.

To prepare receptor containing membranes cells are homogenized in wash buffer (50 mM Tris HCl, 10 mM MgCl₂, 2 mM EGTA, pH 7.4) and centrifuged at 48,000×g for 10 minutes at 4° C. The pellet is re-suspended in wash buffer and the homogenization and centrifugation steps are performed two additional times.

Membrane pellets (containing CRF receptors) are re-suspended in 50 mM Tris buffer pH 7.7 containing 10 mM MgCl₂ and 2 mM EDTA and centrifuged for 10 minutes at 48,000g. Membranes are washed again and brought to a final concentration of 1500 ug/ml in binding buffer (Tris buffer above with 0.1% BSA, 15 mM bacitracin and 0.01 mg/ml aprotinin.). For the binding assay, 100 ul of the membrane preparation are added to 96 well microtube plates containing 100 ul of ¹²⁵I-CRF (SA 2200 Ci/mmol, final concentration of 100 pM) and 50 ul of test compound. Binding is carried out at room temperature for 2 hours. Plates are then harvested on a BRANDEL 96 well cell harvester and filters are counted for gamma emissions on a Wallac 1205 BETAPLATE liquid scintillation counter. Non-specific binding is defined by 1 mM cold CRF. IC₅₀ values are calculated with the non-linear curve fitting program RS/1 (BBN Software Products Corp., Cambridge, Mass.). The binding affinity for the compounds of Formula I expressed as IC₅₀ value, generally ranges from about 0.5 nanomolar to about 10 micromolar. Preferred compounds of Formula I exhibit IC₅₀ values of less than or equal to 1.5 micromolar, more preferred compounds of Formula I exhibit IC₅₀ values of less than 500 nanomolar, still more preferred compounds of Formula I exhibit IC₅₀ values of less than 100 nanomolar, and most preferred compound of Formula I exhibit IC₅₀ values of less than 10 nanomolar. The compounds shown in Examples have been tested in this assay and found to exhibit IC₅₀ values of less than or equal to 4 micromolar.

### Example 12

### Preparation of Radiolabeled Probe Compounds of the Invention

The compounds of the invention are prepared as radiolabeled probes by carrying out their synthesis using precursors comprising at least one atom that is a radioisotope. The radioisotope is preferably selected from of at least one of carbon (preferably ¹⁴C), hydrogen (preferably 3H), sulfur (preferably ³⁵S), or iodine (preferably ¹²⁵I). Such radiolabeled probes are conveniently synthesized by a radioisotope supplier specializing in custom synthesis of radiolabeled probe compounds. Such suppliers include Amersham Corporation, Arlington Heights, Ill.; Cambridge Isotope Laboratories, Inc. Andover, Mass.; SRI International, Menlo Park, Calif.; Wizard Laboratories, West Sacramento, Calif.; ChemSyn Laboratories, Lexena, Kans.; American Radiolabeled Chemicals, Inc., St. Louis, Mo.; and Moravek Biochemicals Inc., Brea, Calif.

Tritium labeled probe compounds are also conveniently prepared catalytically via platinum-catalyzed exchange in tritiated acetic acid, acid-catalyzed exchange in tritiated trifluoroacetic acid, or heterogeneous-catalyzed exchange with tritium gas. Such preparations are also conveniently carried out as a custom radiolabeling by any of the suppliers listed in the preceding paragraph using the compound of the invention as substrate. In addition, certain precursors may be subjected to tritium-halogen exchange with tritium gas, tritium gas reduction of unsaturated bonds, or reduction using sodium borotritide, as appropriate.

### Example 13

### Receptor Autoradiography

Receptor autoradiography (receptor mapping) is carried out in vitro as described by Kuhar in sections 8.1.1 to 8.1.9 of Current Protocols in Pharmacology (1998) John Wiley & Sons, New York, using radiolabeled compounds of the invention prepared as described in the preceding Examples.

### Example 14

### Additional Aspects of Preferred Compounds of the Invention

The most preferred compounds of the invention are suitable for pharmaceutical use in treating human patients. Accordingly, such preferred compounds are non-toxic. They do not exhibit single or multiple dose acute or long-term toxicity, mutagenicity (e.g., as determined in a bacterial reverse mutation assay such as an Ames test), teratogenicity, tumorogenicity, or the like, and rarely trigger adverse effects (side effects) when administered at therapeutically effective dosages.

Preferably, administration of such preferred compounds of the invention at certain doses (i.e., doses yielding therapeutically effective in vivo concentrations or preferably doses of 10, 50, 100, 150, or 200 mg/kg administered parenterally or prefrerably orally) does not result in prolongation of heart QT intervals (i.e., as determined by electrocardiography, e.g., in guinea pigs, minipigs or dogs). When administered daily for 5 or preferably ten days, such doses of such preferred compounds also do not cause liver enlargement resulting in an increase of liver to body weight ratio of more than 100%, preferably not more than 75% and more preferably not more than 50% over matched controls in laboratory rodents (e.g., mice or rats). In another aspect such doses of such preferred compounds also preferably do not cause liver enlargement resulting in an increase of liver to body weight ratio of more than 50%, preferably preferably not more than 25%, and more preferably not more than 10% over matched untreated controls in dogs or other non-rodent mammals.

In yet another aspect such doses of such preferred compounds also preferably do not promote the release of liver enzymes (e.g., ALT, LDH, or AST) from hepatocytes in vivo. Preferably such doses do not elevate serum levels of such enzymes by more than 100%, preferably not by more than 75% and more preferably not by more than 50% over matched untreated controls in laboratory rodents. Similarly, concentrations (in culture media or other such solutions that are contacted and incubated with cells in vitro) equivalent to two, fold, preferably five-fold, and most preferably ten-fold the minimum in vivo therapeutic concentration do not cause release of any of such liver enzymes from hepatocytes into culture medium in vitro above baseline levels seen in media from untreated cells.

Because side effects are often due to undesirable receptor activation or antagonism, preferred compounds of the invention exert their receptor-modulatory effects with high selectivity. This means that they do not bind to certain other receptors (other than CRF receptors) with high affinity, but rather only bind to, activate, or inhibit the activity of such other receptors with affinity constants of greater than 100 nanomolar, preferably greater than 1 micromolar, more preferably greater than 10 micromolar and most preferably greater than 100 micromolar. Such receptors preferably are selected from the group including ion channel receptors, including sodium ion channel receptors, neurotransmitter receptors such as alpha- and beta-adrenergic receptors, muscarinic receptors (particularly m1, m2, and m3 receptors), dopamine receptors, and metabotropic glutamate receptors; and also include histamine receptors and cytokine receptors, e.g., interleukin receptors, particularly IL-8 receptors. The group of other receptors to which preferred compounds do not bind with high affinity also includes GABA_{A} receptors, bioactive peptide receptors (including NPY, GLP-1, VIP receptors), neurokinin receptors, bradykinin receptors (e.g., BK1 receptors and BK2 receptors), neurokinin receptors, particularly NK-3 receptors, and hormone receptors (including thyrotropin releasing hormone receptors and melanocyte-concentrating hormone receptors). Preferably the Kᵢ value of a compound of the invention is 100 fold less at CRF1 receptors than at any other membrane bound receptor. Furthermore, preferred compounds of the invention do not exhibit Kᵢ values of less than 4 micromolar at BK-1 receptors, BK-2 receptors, GABAA receptors, GLP-1 receptors, or NK-3 receptors. Radioligand binding assays or other types of assays may be used to determine if a compound of the invention binds with high affinity to a membrane-bound receptor other than a CRF-1 receptor. For example an assay for GLP-1 receptor activation is disclosed in U.S. Pat. No. 6,271,241, which is hereby incorporated by reference at columns 23-24 for its teachings regarding assays for GLP-1 receptor activation. A radioligand binding assay useful for determining the Kᵢ value of a compound at BK-2 receptors is disclosed in U.S. patent application No. 09/540,580, which is hereby incorporated by reference at pages 47-49 for its teachings regarding assays for BK-2 receptor binding. A radioligand binding assay useful for determining the Kᵢ value of a compound at GABA_{A} receptors is disclosed in U.S. patent application No. 09/709,887, which is hereby incorporated by reference at pages 60-62 for its teachings regarding GABA_{A} receptor binding assays.

### Example 14a

### Absence of Sodium Ion Channel Activity

Preferred compounds of the invention do not exhibit activity as sodium ion channel blockers. Sodium channel activity may be measured a standard in vitro sodium channel binding assays such as the assay given by Brown et al. (J. Neurosci. 1986, 265, 17995-18004). Preferred compounds of the invention exhibit less than 15 percent inhibition, and more preferably less than 10 percent inhibition, of sodium channel specific ligand binding when present at a concentration of 4 uM. The sodium ion channel specific ligand used may be labeled batrachotoxinin, tetrodotoxin, or saxitoxin. Such assays, including the assay of Brown referred to above, are performed as a commercial service by CEREP, Inc., Redmond, Wa.

Alternatively, sodium ion channel activity may be measured in vivo in an assay of anti-epileptic activity. Anti-epileptic activity of compounds may be measured by the ability of the compounds to inhibit hind limb extension in the supra maximal electro shock model. Male Han Wistar rats (150-200mg) are dosed i.p. with a suspension of 1 to 20 mg of test compound in 0.25% methylcellulose 2 hours. prior to test. A visual observation is carried out just prior to testing for the presence of ataxia. Using auricular electrodes a current of 200 mA, duration 200 millisec, is applied and the presence or absence of hind limb extension is noted. Preferred compounds of the invention do not exhibit significant anti-epileptic activity at the p<0.1 level of significance or more preferably at the p<0.05 level of significance as measured using a standard parametric assay of statistical significance such as a student's T test.

### Example 14b

### Microsomal in vitro Half-life

Compound half-life values (t_{½} values) may be determined via the following standard liver microsomal half-life assay. Pooled Human liver microsomes are obtained from XenoTech LLC, 3800 Cambridge St. Kansas's City, Kans., 66103 (catalog #H0610). Such liver microsomes may also be obtained from In Vitro Technologies, 1450 South Rolling Road, Baltamore, Md. 21227, or from Tissue Transformation Technologies, Edison Corporate Center, 175 May Street, Suite 600, Edison, N.J. 08837. Reactions are preformed as follows:

Reagents:

Phosphate buffer: 19 mL 0.1 M NaH₂PO₄, 81 mL 0.1 Na₂HPO₄, adjusted to pH 7.4 with H₃PO₄.

CoFactor Mixture: 16.2 mg NADP, 45.4 mg Glucose-6-phosphate in 4 mL 100 mM MgCl₂.

Glucose-6-phosphate dehydrogenase: 214.3 ul glucose-6-phosphate dehydrogenase suspension (Boehringer-Manheim catalog no. 0737224, distributed by Roche Molecular Biochemicals, 9115 Hague Road, P.O. Box 50414, Indianapolis, Ind. 46250) is diluted into 1285.7 ul distilled water.

Starting Reaction Mixture: 3 mL CoFactor Mixture, 1.2 mL Glucose-6-phosphate dehydrogenase.

Reaction:

6 test reactions are prepared, each containing 25 ul microsomes, 5 ul of a 100 uM solution of test compound, and 399 ul 0.1 M phosphate buffer. A seventh reaction is prepared as a positive control containing 25 ul microsomes, 399 ul 0.1 M phosphate buffer, and 5 ul of a 100 uM solution of a compound with known metabolic properties (e.g. DIAZEPAM or CLOZEPINE). Reactions are preincubated at 39° C. for 10 minutes. 71 ul Starting Reaction Mixture is added to 5 of the 6 test reactions and to the positive control, 71 ul 100 mM MgCl₂ is added to the sixth test reaction, which is used as a negative control. At each time point (0, 1, 3, 5, and 10 minutes) 75 ul of each reaction mix is pipetted into a well of a 96-well deep-well plate containing 75 ul ice-cold acetonitrile. Samples are vortexed and centrifuged 10 minutes at 3500 rpm (Sorval T 6000D centrifuge, H1000B rotor). 75 ul of supernatant from each reaction is transferred to a well of a 96-well plate containing 150 ul of a 0.5 uM solution of a compound with a known LCMS profile (internal standard) per well. LCMS analysis of each sample is carried out and the amount of unmetabolized test compound is measured as AUC, compound concentration vs time is plotted, and the t_{½} value of the test compound is extrapolated.

Preferred compounds of the invention exhibit in vitro t_{½} values of greater than 10 minutes and less than 4 hours. Most preferred compounds of the invention exhibit in vitro t_{½} values of between 30 minutes and 1 hour in human liver microsomes.

### Example 14c

### MDCK Toxicity Assay

Compounds causing acute cytotoxicity will decrease ATP production by Madin Darby canine kidney (MDCK) cells in the following assay.

MDCK cells, ATCC no. CCL-34 (American Type Culture Collection, Manassas, Va.) are maintained in sterile conditions following the instructions in the ATCC production information sheet. The PACKARD, (Meriden, Conn.) ATP-LITE-M Luminescent ATP detection kit, product no. 6016941, allows measurement ATP production in MDCK cells.

Prior to assay 1 ul of test compound or control sample is pipetted into PACKARD (Meriden, Conn.) clear bottom 96-well plates. Test compounds and control samples are diluted in DMSO to give final concentration in the assay of 10 micromolar, 100 micromolar, or 200 micromolar. Control samples are drug or other compounds having known toxicity properties.

Confluent MDCK cells are trypsinized, harvested, and diluted to a concentration of 0.1×10⁶ cells/ ml with warm (37° C.) VITACELL Minimum Essential Medium Eagle (ATCC catalog #30-2003). 100ul of cells in medium is pipetted into each of all but five wells of each 96-well plate. Warm medium without cells (100 ul) is pipetted in the remaining five wells of each plate to provide standard curve control wells. These wells, to which no cells are added, are used to determine the standard curve. The plates are then incubated at 37° C. under 95% O₂, 5% CO₂ for 2 hours with constant shaking. After incubation, 50 ul of mammalian cell lysis solution is added per well, the wells are covered with PACKARD TOPSEAL stickers, and plates are shaken at approximately 700 rpm on a suitable shaker for 2 minutes.

During the incubation, PACKARD ATP LITE-M reagents are allowed to equilibrate to room temperature. Once equilibrated the lyophilized substrate solution is reconstituted in 5.5 mls of substrate buffer solution (from kit). Lyophilized ATP standard solution is reconstituted in deionized water to give a 10 mM stock. For the five control wells, 10 ul of serially diluted PACKARD standard is added to each of the five standard curve control wells to yield a final concentration in each subsequent well of 200 nM, 100 nM, 50 nM, 25 nM, and 12.5 nM.

PACKARD substrate solution (50 ul) is added to all wells. Wells are covered with PACKARD TOPSEAL stickers, and plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter, e.g. PACKARD TOPCOUNT Microplate Scintillation and Luminescense Counter or TECAN SPECTRAFLUOR PLUS.

Luminescence values at each drug concentration are compared to the values computed from the standard curve for that concentration. Preferred test compounds exhibit luminescence values 80% or more of the standard, or preferably 90% or more of the standard, when a 10 micromolar (uM) concentration of the test compound is used. When a 100 uM concentration of the test compound is used, preferred test compounds exhibit luminescence values 50% or more of the standard, or more preferably 80% or more of the standard.

What is claimed is:

1. A compound of the following Formula I: 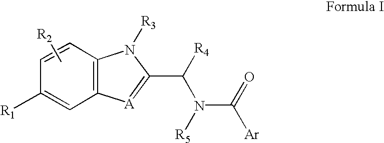
or a pharmaceutically acceptable salt thereof, wherein
A is nitrogen or optionally substituted CH;
R₁ is hydrogen, optionally substituted alkyl, optionally substituted haloalkoxy, optionally substituted haloalkyl, halogen, hydroxy, cyano, amino, nitro, XR_{A}, C₁-C₂alkyl-XR_{A}, Y or C₁-C₂alkyl-Y;
R₂ represents from 0 to 3 substituents independently selected from halogen, hydroxy, cyano, amino, optionally substituted alkyl, optionally substituted alkoxy, optionally substituted haloalkyl, optionally substituted haloalkoxy, optionally substituted hydroxyalkyl, and optionally substituted mono- or di-alkylamino;
R₃ represents an optionally substituted alkyl group; or
R₃ represents a saturated, partially unsaturated, or aromatic ring having from 5 to 7 ring atoms, 0, 1, or 2 ring atoms chosen from oxygen and nitrogen, with remaining ring atoms being carbon, which saturated, partially unsaturated, or aromatic ring is unsubstituted or substituted;
R₄ represents hydrogen or optionally substituted alkyl;
R₅ represents optionally substituted alkyl; or
R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon;
Ar represents phenyl or a heteroaryl group of 5 to 7 ring atoms, 0, 1, or 2 ring atoms chosen from oxygen, nitrogen, and sulfur, with remaining ring atoms being carbon, Ar is substituted ortho to the point of attachment of Ar in Formula I by R₆ and is optionally substituted by 1 or more of R₇;
R₆ represents halogen, hydroxy, cyano, amino, XR_{A}, C₁-C₂alkyl-XR_{A}, or Y;
R₇ is independently selected at each occurrence from hydroxy, cyano, amino, XR_{A}, C₁-C₂alkyl-XR_{A}, and Y;
X is independently selected at each occurrence from the group consisting of a bond, —CH₂—, —CHR_{B}—, —O—, —C(═O)—, —C(═O)O—,—OC(═O)—, —S(O),—, —NH—, —NR_{B}—, —C(═O)NH—, —C(═O)NR_{B}—, —S(O)ₙNH—, —S(O)ₙNR_{B}—, —NHC(═O)—, —NR_{B}C(═O)—, —NHS(O),—, and —NR_{B}S(O)ₙ—;
R_{A} and R_{B} are independently selected at each occurrence from: hydrogen, and optionally substituted straight, branched, and cyclic alkyl groups containing zero or one or more double or triple bonds;
n is independently selected at each occurrence from 0, 1, and 2; and
Y and Z are independently selected at each occurrence from: saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(CI-C₆alkyl), C₁-C₆haloalkyl, C₁-C₆haloalkoxy, and mono- or di-(C₁-C₆)alkylamino.

2. A compound of of the following Formula II: 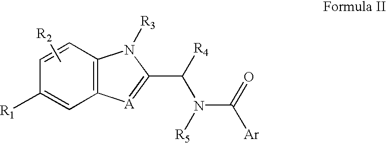
or a pharmaceutically acceptable salt thereof, wherein
A is nitrogen, CH, or C₁-C₆alkyl;
R₁ is hydrogen, optionally substituted alkyl, optionally substituted haloalkoxy, optionally substituted haloalkyl, halogen, hydroxy, cyano, amino, nitro, XR_{A}, C₁-C₂alkyl-XR_{A}, Y or C₁-C₂alkyl-Y;
R₂ represents from 0 to 3 substituents independently selected from halogen, hydroxy, cyano, amino, optionally substituted alkyl, optionally substituted alkoxy, optionally substituted haloalkyl, optionally substituted haloalkoxy, optionally substituted hydroxyalkyl, and optionally substituted mono- or di-alkylamino;
R₃ represents a branched C₃-C₁₀ alkyl group which is optionally substituted by 1 or more substituents independently chosen from halogen, oxo, hydroxy, cyano, amino, C₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; or
R₃ represents a saturated, partially unsaturated, or aromatic ring having from 5 to 7 ring atoms, 0, 1, or 2 ring atoms chosen from oxygen and nitrogen, with remaining ring atoms being carbon, which saturated, partially unsaturated, or aromatic ring is unsubstituted or substituted with one or more substituents independently selected from halogen, oxo, hydroxy, amino, cyano, XR_{A}, C₁-C₆alkyl, C₁-C₆alkyl substituted by XR_{A}, C₁-C₆alkoxy,
C₁-C₆alkoxy substituted by XR_{A}, C₁-C₆haloalkyl, C₁-C₆haloalkoxy, mono- or di-(C₁-C₆)alkylamino, and Y;
R₄ represents hydrogen or optionally substituted C₁-C₆ alkyl;
R₅ represents branched C₃-C₁₀ alkyl which is unsubstituted or substituted by 1 to 4 groups independently chosen from hydroxy, cyano, amino, oxo, XR_{A}, and Y; or
R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon; said saturated or partially unsaturated ring is unsubstituted or substituted by 1 to 3 substituents independently chosen from halogen, hydroxy, amino, C₁-C₆alkoxy, C₁-C₆alkyl, C₁-C₆haloalkyl, C₁-C₆haloalkoxy, C₁-C₆hydroxyalkyl, and mono- or di-(C₁-C₆)alkylamino;
Ar represents phenyl or a heteroaryl group of 5 to 7 ring atoms, 0, 1, or 2 ring atoms chosen from oxygen, nitrogen, and sulfur, with remaining ring atoms being carbon, Ar is substituted ortho to the point of attachment of Ar in Formula I by R₆ and is optionally substituted by 1 or more of R₇;
R₆ represents halogen, hydroxy, cyano, amino, XR_{A}, C₁-C₂alkyl-XR_{A}, or Y;
R₇ is independently selected at each occurrence from hydroxy, cyano, amino, XR_{A}, C₁-C₂alkyl-XR_{A}, and Y;
X is independently selected at each occurrence from the group consisting of a bond, —CH₂—, —CHR_{B}—, —O—, —C(═O)—, —C(═O)O—, —OC(═O)—, —S(O)ₙ—, —NH—, —NR_{B}—, —C(═O)NH—, —C(═O)NR_{B}—, —S(O)ₙNH—, —S(O)ₙNR_{B}—, —NHC(═O)—, —NR_{B}C(═O)—, —NHS(O)ₙ—, and —NR_{B}S(O)ₙ—;
R_{A} and R_{B} are independently selected at each occurrence from:
hydrogen, and
straight, branched, and cyclic alkyl groups, and (cycloalkyl)alkyl groups, said straight, branched, and cyclic alkyl groups, and (cycloalkyl)alkyl groups consisting of 1 to 8 carbon atoms, and containing zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
n is independently selected at each occurrence from 0, 1, and 2; and
Y and Z are independently selected at each occurrence from: saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di-(C₁-C₆)alkylamino.

3. A compound of the following Formula III: 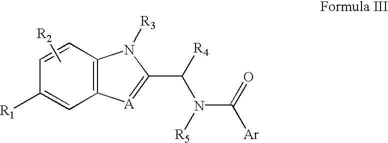
or a pharmaceutically acceptable salt thereof, wherein
A is nitrogen, CH, or C-C₁-C₆alkyl;
R₁ is hydrogen, halogen, hydroxy, cyano, amino, nitro, XR_{A}, C₁-C₂alkyl-XR_{A}, Y or C₁-C₂alkyl-Y;
R₂ represents from 0 to 3 substituents independently selected from halogen, hydroxy, cyano, amino, C₁-C₆ alkyl, C₁-C₆ alkoxy, C₁-C₆ haloalkyl, C₁-C₆ haloalkoxy, C₁-C₆ hydroxyalkyl, and C₁-C₆ mono- or di-alkylamino;
R₃ represents a branched C₃-C₁₀ alkyl group which is optionally substituted by 1 or more substituents independently chosen from halogen, oxo, hydroxy, cyano, amino, C₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; or
R₃ represents a saturated, partially unsaturated, or aromatic ring having from 5 to 7 ring atoms, 0, 1, or 2 ring atoms chosen from oxygen and nitrogen, with remaining ring atoms being carbon, which saturated, partially unsaturated, or aromatic ring is unsubstituted or substituted with one or more substituents independently selected from halogen, oxo, hydroxy, amino, cyano, XR_{A}, C₁-C₆alkyl, C₁-C₆alkyl substituted by XR_{A}, C₁-C₆alkoxy, C₁-C₆alkoxy substituted by XR_{A}, C₁-C₆haloalkyl, C₁-C₆haloalkoxy, mono- or di-(C₁-C₆)alkylamino, and Y;
R₄ represents hydrogen or C₁-C₆ alkyl;
R₅ represents branched C₃-C₁₀ alkyl which is unsubstituted or substituted by 1 to 4 groups independently chosen from hydroxy, cyano, amino, oxo, XR_{A}, and Y; or
R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon; said saturated or partially unsaturated ring is unsubstituted or substituted by 1 to 3 substituents independently chosen from halogen, hydroxy, amino, C₁-C₆alkoxy, C₁-C₆alkyl, C₁-C₆haloalkyl, C₁-C₆haloalkoxy, C₁-C₆hydroxyalkyl, and mono- or di-(C₁-C₆)alkylamino;
Ar represents phenyl or a heteroaryl group of 5 to 7 ring atoms, 0, 1, or 2 ring atoms chosen from oxygen, nitrogen, and sulfur, with remaining ring atoms being carbon, Ar is substituted ortho to the point of attachment of Ar in Formula III by R₆ and is optionally substituted by 1 or more of R₇;
R₆ represents halogen, hydroxy, cyano, amino, XR_{A}, C₁-C₂alkyl-XR_{A}, or Y;
R₇ is independently selected at each occurrence from hydroxy, cyano, amino, XR_{A}, C₁-C₂alkyl-XR_{A}, and Y;
X is independently selected at each occurrence from the group consisting of a bond, —CH₂—, —CHR_{B}—, —O—, —C(═O)—, —C(═O)O—, —OC(═O)—, —S(O),—, —NH—, —NR_{B}—, —C(═O)NH—, —C(═O)NR_{B}—, —S(O)ₙNH—, —S(O)ₙNR_{B}—, —NHC(═O)—, —NR_{B}C(═O)—, —NHS(O)ₙ—, and —NR_{B}S(O)ₙ—;
R_{A} and R_{B} are independently selected at each occurrence from:
hydrogen, and
straight, branched, and cyclic alkyl groups, and (cycloalkyl)alkyl groups, said straight, branched, and cyclic alkyl groups, and (cycloalkyl)alkyl groups consisting of 1 to 8 carbon atoms, and containing zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
n is independently selected at each occurrence from 0, 1, and 2; and
Y and Z are independently selected at each occurrence from: saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di-(C₁-C₆)alkylamino.

4. A compound or salt according to claim 3, wherein A is nitrogen.

5. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl.

6. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
Ar represents phenyl, pyridyl, pyrimidinyl, thienyl, pyrrolyl, imidazolyl, pyrazolyl, thiazolyl, oxazolyl, or isoxazolyl, each of which is substituted ortho to the point of attachment in Formula III by R₆ and is optionally substituted by from 1 to 3 of R₇; wherein R₆ and R₇ are as defined in claim 2.

7. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino; and
Ar represents phenyl, pyridyl, pyrimidinyl, thienyl, pyrrolyl, imidazolyl, pyrazolyl, thiazolyl, oxazolyl, or isoxazolyl, each of which is substituted ortho to the point of attachment in Formula III by R₆ and is optionally substituted by from 1 to 3 of R₇.

8. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₄ represents hydrogen or methyl;
R₅ represents branched C₃-C₁₀ alkyl; and
Ar represents phenyl or pyridyl, each of which is substituted ortho to the point of attachment in Formula III by R₆ and is optionally substituted by from 1 to 3 of R₇; wherein R₆ and R₇ are as defined in claim 4.

9. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₄ represents hydrogen or methyl;
R₅ represents branched C₃-C₁₀ alkyl;
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkyl; C₁-C₆alkoxyCl-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

10. A compound or salt according to claim 4, wherein:
R₂ represents hydrogen;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₄ represents hydrogen or methyl;
R₅ represents branched C₃-C₁₀ alkyl;
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

11. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents phenyl or pyridyl which is optionally substituted by 1 or 2 substituents independently selected from hydroxy, amino, halogen C₁-C₆alkoxy, C₁-C₆alkyl; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; and
Ar represents phenyl, pyridyl, pyrimidinyl, thienyl, pyrrolyl, imidazolyl, pyrazolyl, thiazolyl, oxazolyl, or isoxazolyl, each of which is substituted ortho to the point of attachment in Formula III by R₆ and is optionally substituted by from 1 to 3 of R₇.

12. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents phenyl or pyridyl which is optionally substituted by 1 or 2 substituents independently selected from hydroxy, amino, halogen C₁-C₆alkoxy, C₁-C₆alkyl; and mono- or di-(C₁-C₆)alkylamino;
R₄ represents hydrogen or methyl;
R₅ represents branched C₃-C₁₀ alkyl; and
Ar represents phenyl or pyridyl, each of which is substituted ortho to the point of attachment in Formula III by R₆ and is optionally substituted by from 1 to 3 of R₇; wherein R₆ and R₇ are as defined in claim 2.

13. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents phenyl which is optionally substituted by 1 or 2 substituents independently selected from hydroxy, amino, halogen, C₁-C₄alkoxy, and C₁-C₄alkyl;
R₄ represents hydrogen or methyl;
R₅ represents branched C₃-C₁₀ alkyl;
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

14. A compound or salt according to claim 4, wherein:
R₂ represents hydrogen;
R₃ represents phenyl which is optionally substituted at the position para to the point of attachment of R₃ in Formula III by C₁-C₂alkoxy or C₁-C₂alkyl;
R₄ represents hydrogen or methyl;
R₅ represents branched C₃-C₁₀ alkyl;
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

15. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon; said saturated or partially unsaturated ring is unsubstituted or substituted by 1 to 3 substituents independently chosen from halogen, hydroxy, amino, C₁-C₆alkoxy, C₁-C₆alkyl; C₁-C₆haloalkyl, C₁-C₆haloalkoxy, C₁-C₆hydroxyalkyl, and mono- or di-(C₁-C₆)alkylamino;
Ar represents phenyl or pyridyl, each of which is substituted ortho to the point of attachment in Formula III by R₆ and is optionally substituted by from 1 to 3 of R₇.

16. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₄ and R₅ are joined to form a saturated ring of from 5 to 8 ring members, said ring members comprising 0 or 1 additional nitrogen atom, with remaining ring members being carbon; said saturated ring is unsubstituted or substituted by 1 to 3 substituents independently chosen from halogen, hydroxy, amino, C₁-C₂alkoxy, C₁-C₂alkyl; C₁-C₂haloalkyl, C₁-C₂haloalkoxy, and mono- or di-(C₁-C₂)alkylamino;
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

17. A compound or salt according to claim 4, wherein:
R₂ represents hydrogen;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₄ and R₅ are joined to form a saturated ring of from 5 to 7 ring members, said ring members comprising 1 nitrogen atom, with remaining ring members being carbon; said saturated ring is unsubstituted or substituted by 1 to 3 substituents independently chosen from halogen, methyl, and methoxy;
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

18. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents phenyl or pyridyl which is optionally substituted by 1 or 2 substituents independently selected from hydroxy, amino, halogen C₁-C₆alkoxy, C₁-C₆alkyl; and mono- or di-(C₁-C₆)alkylamino;
R₄ and R₅ are joined to form a saturated or partially unsaturated ring of from 5 to 8 ring members, said ring members comprising 0 or 1 additional nitrogen atom, 0 or 1 oxygen atom, with remaining ring members being carbon; said saturated or partially unsaturated ring is unsubstituted or substituted by 1 to 3 substituents independently chosen from halogen, hydroxy, amino, C₁-C₆alkoxy, C₁-C₆alkyl; C₁-C₆haloalkyl, C₁-C₆haloalkoxy, C₁-C₆hydroxyalkyl, and mono- or di-(C₁-C₆)alkylamino; and
Ar represents phenyl or pyridyl, each of which is substituted ortho to the point of attachment in Formula III by R₆ and is optionally substituted by from 1 to 3 of R₇; wherein R₆ and R₇ are as defined in claim 4.

19. A compound or salt according to claim 4, wherein:
R₂ represents from 0 to 3 substituents independently selected from halogen, hydroxy, C₁-C₂alkyl, C₁-C₂alkoxy, C₁-C₂haloalkyl, C₁-C₂haloalkoxy, and C₁-C₂hydroxyalkyl;
R₃ represents phenyl which is optionally substituted by 1 or 2 substituents independently selected from hydroxy, amino, halogen, C₁-C₄alkoxy, and C₁-C₄alkyl;
R₄ and R₅ are joined to form a saturated ring of from 5 to 8 ring members, said ring members comprising 0 or 1 additional nitrogen atom, with remaining ring members being carbon; said saturated ring is unsubstituted or substituted by 1 to 3 substituents independently chosen from halogen, hydroxy, amino, C₁-C₂alkoxy, C₁-C₂alkyl; C₁-C₂haloalkyl, C₁-C₂haloalkoxy, and mono- or di-(C₁-C₂)alkylamino;
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

20. A compound or salt according to claim 4, wherein:
R₂ represents hydrogen;
R₃ represents phenyl which is optionally substituted at the position para to the point of attachment of R₃ in Formula III by C₁-C₂alkoxy or C₁-C₂alkyl;
R₄ and R₅ are joined to form a saturated ring of from 5 to 7 ring members, said ring members comprising 1 nitrogen atom, with remaining ring members being carbon; said saturated ring is unsubstituted or substituted by 1 to 3 substituents independently chosen from halogen, methyl, and methoxy;
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

21. A compound or salt according to claim 4, wherein
R₁ represents phenyl or pyridyl each of which is optionally substituted with 1 to 5 R₇; or
R₁ represents C₁-C₂alkyl-Y, amino, mono or di(C₁-C₆alkyl)amino, mono or di(C₁-C₆alkyl)amino-C₁-C₆alkyl each of which may be optionally substituted with one or two C₁-C₆alkyl, C₁-C₆alkoxy, C₁-C₆alkoxyC₁-C₆alkyl, hydroxy or amino; and
Y is selected from saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di- (C₁-C₆)alkylamino.

22. A compound or salt according to claim 4, wherein
R₁ represents phenyl or pyridyl each of which is optionally substituted with 1 to 3 R₇; or
R₁ represents C₁-C₂alkyl-Y, amino, mono or di(C₁-C₆alkyl)amino, mono or di(C₁-C₆alkyl)amino-C₁-C₄alkyl each of which may be optionally substituted with one or two C₁-C₆alkyl, C₁-C₆alkoxy, C₁-C₆alkoxyC₁-C₆alkyl, hydroxy or amino;
R₃ represents phenyl, which is optionally substituted by 1 or 2 substituents independently, selected from hydroxy, amino, C₁-C₄alkyl, and C₁-C₄alkoxy;
R₅ represents a branched C₃-C₁₀ alkyl group which is optionally substituted by 1 or more substituents independently chosen from hydroxy, amino, C₁-C₆alkoxy, and mono- or di- (C₁-C₆)alkylamino; and
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

23. A compound or salt according to claim 4, wherein
R₁ represents phenyl or pyridyl each of which is optionally substituted with 1 to 3 R₇; or
R₁ represents C₁-C₂alkyl-Y, amino, mono or di(C₁-C₆alkyl)amino, mono or di(C₁-C₆alkyl)amino-C₁-C₄alkyl each of which may be optionally substituted with one or two C₁-C₆alkyl, C₁-C₆alkoxy, C₁-C₆alkoxyC₁-C₆alkyl, hydroxy or amino;
R₃ represents phenyl, which is optionally substituted by 1 or 2 substituents independently, selected from hydroxy, amino, C₁-C₄alkyl, and C₁-C₄alkoxy;
R₄ and R₅ are joined to form a saturated ring of from 5 to 7 ring members, said ring members comprising 1 nitrogen atom, with remaining ring members being carbon; said saturated ring is unsubstituted or substituted by 1 to 3 substituents independently chosen from halogen, methyl, and methoxy; and
Ar represents phenyl or pyridyl, each of which is substituted ortho to the point of attachment in Formulae III by R₆ and is optionally substituted by from 1 to 3 of R₇; wherein:
R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

24. A compound or salt according to claim 4, wherein
R₁ represents phenyl or pyridyl each of which is optionally substituted with 1 to 3 R₇; or
R₁ represents C₁-C₂alkyl-Y, amino, mono or di(C₁-C₆alkyl)amino, mono or di(C₁-C₆alkyl)amino-C₁-C₄alkyl each of which may be optionally substituted with one or two C₁-C₆alkyl, C₁-C₆alkoxy, C₁-C₆alkoxyC₁-C₆alkyl, hydroxy or amino;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₅ represents a branched C₃-C₁₀ alkyl group which is optionally substituted by 1 or more substituents independently chosen from hydroxy, amino, C₁-C₆alkoxy, and mono- or di- (C₁-C₆)alkylamino; and
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

25. A compound or salt according to claim 4, wherein
R₁ represents phenyl or pyridyl each of which is optionally substituted with 1 to 3 R₇; or
R₁ represents C₁-C₂alkyl-Y, amino, mono or di(C₁-C₆alkyl)amino, mono or di(C₁-C₆alkyl)amino-C₁-C₄alkyl each of which may be optionally substituted with one or two C₁-C₆alkyl, C₁-C₆alkoxy, C₁-C₆alkoxyC₁-C₆alkyl, hydroxy or amino;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₄ and R₅ are joined to form a saturated ring of from 5 to 7 ring members, said ring members comprising 1 nitrogen atom, with remaining ring members being carbon; said saturated ring is unsubstituted or substituted by 1 to 3 substituents independently chosen from halogen, methyl, and methoxy; and
Ar represents phenyl or pyridyl, each of which is substituted ortho to the point of attachment in Formula III by R₆ and is optionally substituted by from 1 to 3 of R₇; wherein:
R₆ and R₇ are as independently chosen from hydroxy, amino, halogen C₁-C₆alkoxy, C₁-C₆alkyl; C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

26. A compound according to Formula IV: 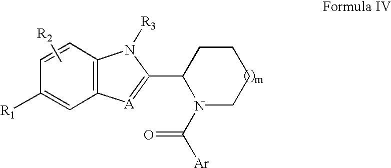
or a pharmaceutically acceptable salt thereof, wherein
A is nitrogen, CH, or C-C₁-C₆alkyl;
R₁ is hydrogen, halogen, hydroxy, cyano, amino, nitro, XR_{A}, C₁-C₂alkyl-XR_{A}, Y or C₁-C₂alkyl-Y;
R₂ represents from 0 to 3 substituents independently selected from halogen, hydroxy, cyano, amino, C₁-C₆alkyl, C₁-C₆alkoxy, C₁-C₆haloalkyl, C₁-C₆haloalkoxy, C₁-C₆hydroxyalkyl, and mono- or di-(C₁-C₆)alkylamino;
R₃ represents a branched C₃-C₁₀ alkyl group which is optionally substituted by 1 or more substituents independently chosen from halogen, oxo, hydroxy, cyano, amino, C₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; or
R₃ represents a saturated, partially unsaturated, or aromatic ring having from 5 to 7 ring atoms, 0, 1, or 2 ring atoms chosen from oxygen and nitrogen, with remaining ring atoms being carbon, which saturated, partially unsaturated, or aromatic ring is unsubstituted or substituted with one or more substituents independently selected from halogen, oxo, hydroxy, amino, cyano, XR_{A}, C₁-C₆alkyl, C₁-C₆alkyl substituted by XR_{A}, C₁-C₆alkoxy, C₁-C₆alkoxy substituted by XR_{A}, C₁-C₆haloalkyl, C₁-C₆haloalkoxy, mono- or di-(C₁-C₆)alkylamino, and Y;
Ar represents phenyl or a heteroaryl group of 5 to 7 ring atoms, 0, 1, or 2 ring atoms chosen from oxygen, nitrogen, and sulfur, with remaining ring atoms being carbon, Ar is substituted ortho to the point of attachment of Ar in Formula IV by R₆ and is optionally substituted by 1 or more of R₇;
R₆ represents halogen, hydroxy, cyano, amino, XR_{A}, C₁-C₂alkyl-XR_{A}, or Y;
R₇ is independently selected at each occurrence from hydroxy, cyano, amino, XR_{A}, C₁-C₂alkyl-XR_{A}, and Y;
X is independently selected at each occurrence from the group consisting of a bond, —CH₂—, —CHR_{B}—, —O—, —C(═O)—, —C(═O)O—, —OC(═O)—, —S(O)ₙ—, —NH—, —NR_{B}—, —C(═O)NH—, —C(═O)NR_{B}—, —S(O)ₙNH—, —S(O)ₙNR_{B}—, —NHC(═O)—, —NR_{B}C(═O)—, —NHS(O)ₙ—, and —NR_{B}S(O)ₙ—;
R_{A} and R_{B} are independently selected at each occurrence from:
hydrogen, and
straight, branched, and cyclic alkyl groups, and (cycloalkyl)alkyl groups, said straight, branched, and cyclic alkyl groups, and (cycloalkyl)alkyl groups consisting of 1 to 8 carbon atoms, and containing zero or one or more double or triple bonds, each of which 1 to 8 carbon atoms may be further substituted with one or more substituent(s) independently selected from oxo, hydroxy, halogen, cyano, amino, C₁-C₆alkoxy, —NH(C₁-C₆alkyl), —N(C₁-C₆alkyl)(C₁-C₆alkyl), —NHC(═O)(C₁-C₆alkyl), —N(C₁-C₆alkyl)C(═O)(C₁-C₆alkyl), —NHS(O)ₙ(C₁-C₆alkyl), —S(O)ₙ(C₁-C₆alkyl), —S(O)ₙNH(C₁-C₆alkyl), —S(O)ₙN(C₁-C₆alkyl)(C₁-C₆alkyl), and Z;
m is selected from 0, 1, 2, and 3;
n is independently selected at each occurrence from 0, 1, and 2; and
Y and Z are independently selected at each occurrence from: saturated, partially unsaturated, or aromatic rings having from 5 to 7 ring atoms, 0, 1, or 2 ring atoms chosen from oxygen and nitrogen, with remaining ring atoms being carbon, which rings are unsubstituted or substituted with one or more substituents independently selected from halogen, oxo, hydroxy, amino, cyano, C₁-C₆alkyl, C₁-C₆alkoxy, C₁-C₆alkoxy(C₁-C₆alkyl), C₁-C₆haloalkyl, C₁-C₆haloalkoxy, and mono- or di-(C₁-C₆)alkylamino.

27. A compound according to claim 26 of the following Formula V: 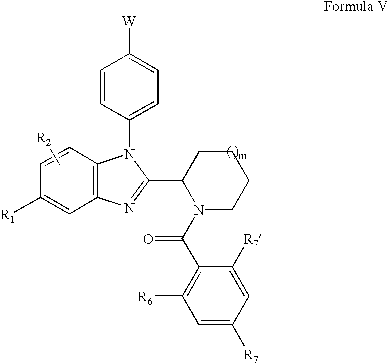
or a pharmaceutically acceptable salt thereof, wherein
R₁ is hydrogen, hydroxy, amino, halogen, C₁-C₆alkoxy, C₁-C₆alkyl, C₁-C₆alkoxyC₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;
R₂ represents from 0 to 3 substituents independently selected from halogen, hydroxy, cyano, amino, C₁-C₂alkyl, C₁-C₂alkoxy, C₁-C₂haloalkyl, C₁-C₂haloalkoxy, and mono- or di-(C₁-C₂)alkylamino;
R₆, R₇, and R_{7′} are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;
m is O or 1; and
W is methyl, ethyl, methoxy, or ethoxy.

28. A compound of Formula VI: 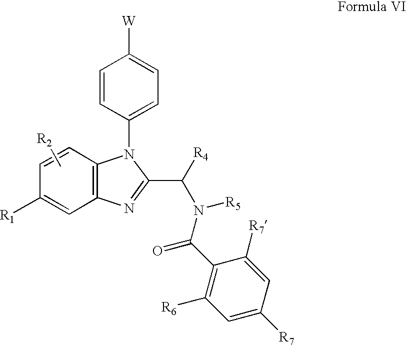
or a pharmaceutically acceptable salt thereof, wherein
R₁ is hydrogen, hydroxy, amino, halogen, C₁-C₆alkoxy, C₁-C₆alkyl, C₁-C₆alkoxyC₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;
R₂ represents from 0 to 3 substituents independently selected from halogen, hydroxy, cyano, amino, C₁-C₂alkyl, C₁-C₂alkoxy, C₁-C₂haloalkyl, C₁-C₂haloalkoxy, and mono- or di-(C₁-C₂)alkylamino;
R₄ is hydrogen or methyl;
R₅ represents branched C₃-C₁₀ alkyl;
R₆, R₇, and R_{7′} are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; and
W is methyl, methoxy, ethyl or ethoxy.

29. A compound of the following Formula VII: 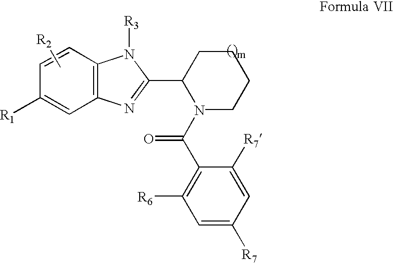
or a pharmaceutically acceptable salt thereof, wherein
R₁ is hydrogen, hydroxy, amino, halogen, C₁-C₆alkoxy, C₁-C₆alkyl, C₁-C₆alkoxyC₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;
R₂ represents from 0 to 3 substituents independently selected from halogen, hydroxy, cyano, amino, C₁-C₂alkyl, C₁-C₂alkoxy, C₁-C₂haloalkyl, C₁-C₂haloalkoxy, and mono- or di-(C₁-C₂)alkylamino;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₆, R₇, and R_{7′} are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino; and
m is O or 1.

30. A compound of Formula VII 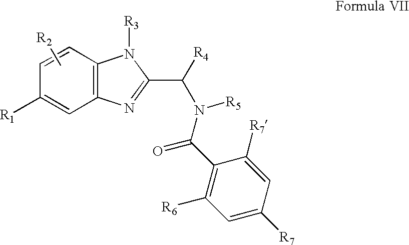
or a pharmaceutically acceptable salt thereof, wherein
R₁ is hydrogen, hydroxy, amino, halogen, C₁-C₆alkoxy, C₁-C₆alkyl, C₁-C₆alkoxyC₁-C₆alkoxy, C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino;
R₂ represents from 0 to 3 substituents independently selected from halogen, hydroxy, cyano, amino, C₁-C₂alkyl, C₁-C₂alkoxy, C₁-C₂haloalkyl, C₁-C₂haloalkoxy, and mono- or di-(C₁-C₂)alkylamino;
R₃ represents a branched C₃-C₁₀ alkyl group which is optionally substituted by 1 or more substituents independently chosen from hydroxy, amino, C₁-C₆alkoxy, and mono- or di- (C₁-C₆)alkylamino;
R₄ is hydrogen or methyl;
R₅ represents branched C₃-C₁₀ alkyl; and
R₆, R₇, and R_{7′} are as independently chosen from hydroxy, amino, halogen C₁-C₆alkoxy, C₁-C₆alkoxyC₁-C₆alkoxy; C₃-C₇cycloalkyl, C₃-C₇cycloalkoxy, C₁-C₆haloalkyl, C₁-C₆haloalkoxy, C₁-C₆hydroxyalkyl, and mono- or di-(C₁-C₆)alkylamino.

31. A compound or salt according to claim 3 wherein, in a standard in vitro CRF receptor binding assay the compound exhibits an IC₅₀ value for CRF receptors of less than or equal to 1 micromolar.

32. A compound or salt according to claim 3 wherein, in a standard in vitro CRF receptor binding assay the compound exhibits an IC₅₀ value for CRF receptors of less than or equal to 100 nanomolar.

33. A compound or salt according to claim 3 wherein, in a standard in vitro CRF receptor binding assay, the compound exhibits an IC₅₀ value for CRF receptors of less than or equal to 10 nanomolar.

34. A method for treating an anxiety disorder, a stress-related disorder, or an eating disorder, comprising administering to a patient in need of such treatment a therapeutically effective amount of a compound or salt according to claim 3.

35. A method for treating an depression or bipolar disorder, comprising administering to a patient in need of such treatment a therapeutically effective amount of a compound or salt according to claim 3.

36. A method for treating anorexia nervosa, bulimia nervosa, or obesity, comprising administering to a patient in need of such treatment a therapeutically effective amount of a compound or salt according to claim 3.

37. A pharmaceutical composition comprising a pharmaceutically acceptable carrier and a compound or salt of claim 3.

38. A package comprising a pharmaceutical composition of claim 37 in a container and further comprising indicia comprising at least one of:
instructions for using the composition to treat a patient suffering from an anxiety disorder, or
instructions for using the composition to treat a patient suffering from a stress-related disorder, or
instructions for using the composition to treat a patient suffering from an eating disorder.

39. A package comprising a pharmaceutical composition of claim 37 in a container and further comprising indicia comprising at least one of: instructions for using the composition to treat a patient suffering from depression or instructions for using the composition to treat a patient suffering from a bipolar disorder.

40. A method for demonstrating the presence of CRF 1 receptors in cell or tissue samples, said method comprising:
preparing a plurality of matched cell or tissue samples,
preparing at least one control sample by contacting under conditions that permit binding of CRF to CRF 1 receptors within cell and tissue samples at least one of the matched cell or tissue samples with a control solution comprising a detectably-labeled preparation of a selected compound or salt of claim 3 at a first measured molar concentration, said control solution further comprising an unlabelled preparation of the selected compound or salt at a second measured molar concentration, which second measured concentration is greater than said first measured concentration,
preparing at least one experimental sample by contacting under conditions that permit binding of CRF to CRF 1 receptors within cell and tissue samples at least one of the matched cell or tissue samples with an experimental solution comprising the detectably-labeled preparation of a the selected compound or salt at the first measured molar concentration, said experimental solution not further comprising an unlabelled preparation of any compound or salt of claim 3 at a concentration greater than or equal to said first measured concentration;
washing the at least one control sample to remove unbound selected compound or salt to produce at least one washed control sample;
washing the at least one experimental sample to remove unbound selected compound or salt to produce at least one washed experimental sample;
measuring the amount of detectable label of any remaining bound detectably-labeled selected compound or salt in the at least one washed control sample;
measuring the amount detectable label of any remaining bound detectably-labeled selected compound or salt in the at least one washed experimental sample;
comparing the amount of detectable label measured in each of the at least one washed experimental sample to the amount of detectable label measured in each of the at least one washed control sample
wherein, a comparison that indicates the detection of a greater amount of detectable label in the at least one washed experimental sample than is detected in any of the at least one washed control samples demonstrates the presence of CRF 1 receptors in that experimental sample.

41. The method of claim 40 wherein the compound is radiolabeled.

42. The method of claim 41 wherein the detection is accomplished using autoradiography.

43. A method of inhibiting the binding of CRF to a CRF1 Receptor, which method comprises:
contacting a solution comprising CRF and a compound or salt of claim 3 with a cell expressing the CRF receptor, wherein the compound or salt is present in the solution at a concentration sufficient to inhibit in vitro CRF binding to 1MR32 cells.

44. A method for altering the signal-transducing activity of CRF 1 receptors, said method comprising contacting cells expressing such receptors with a solution comprising a compound according to claim 3 at a concentration sufficient to detectably alter the electrophysiology of the cell, wherein a detectable alteration of the electrophysiology of the cell indicates an alteration of the signal-transducing activity of CRF 1 receptors.
